(19) United States
(12) Patent Application Publication
(10) Pub. No.: US 2002/0037279 A1
(43) Pub. Date: Mar. 28, 2002

(54) DELIVERY OF BIOACTIVE COMPOUNDS TO AN ORGANISM

(76) Inventor: HERMAN H. VANDENBURGH, PROVIDENCE, RI (US)

Correspondence Address: NIXON PEABODY LLP, ATTENTION: DAVID RESNICK, 101 FEDERAL STREET, BOSTON, MA, 02110, US

(21) Appl. No.: 09/118,950

(22) Filed: Jul. 17, 1998

Related U.S. Application Data
(63) Continuation-in-part of application No. 08/896,152, filed on Jul. 17, 1997.
Application No. 08/896,152 is a continuation-in-part of application No. 08/712,111, filed on Sep. 13, 1996, now Pat. No. 5,869,041.

(51) Int. Cl. A61K 48/00; C12Q 1/02; C12N 5/08

(52) U.S. Cl. 424/93.21; 424/93.2; 435/29; 435/320.1; 435/325; 435/455

(57) ABSTRACT

Disclosed herein is a method of delivering a bioactive compound to an organism that involves growing individual cells in vitro under conditions that allow the formation of an organized tissue, at least a subset of the cells containing a foreign DNA sequence which mediates the production of the bioactive compound; and implanting the organized tissue into the organism, whereby the bioactive compound is produced and delivered to the organism. Also disclosed herein is an in vitro method for producing a tissue having in vivo-like gross and cellular morphology that involves providing precursor cells of the tissue; mixing the cells with a solution of extracellular matrix components to create a suspension; placing the suspension in a vessel having a three dimensional geometry approximating the in vivo gross and cellular morphology of the tissue and having attachment surfaces coupled thereto; allowing the suspension to coalesce; and culturing the cells under conditions in which the cells form an organized tissue connected to the attachment surfaces. Also disclosed herein is an apparatus for producing in vitro a tissue having in vivo-like gross and cellular morphology. This apparatus includes a vessel having a three dimensional geometry approximating the in vivo morphology of the tissue and tissue attachment surfaces coupled thereto.

This application is a continuation-in-part of co-pending U.S. Ser. No. 08/896,152, filed July 17, 1997 which is a continuation-in-part of co-pending U.S. Ser. No. 08/712,111, filed Sep. 13, 1996.

### BACKGROUND OF THE INVENTION

This invention relates to the delivery of bioactive compounds to an organism, and in particular to methods and apparatus for the delivery of bioactive compounds by implanting into the organism an organized tissue producing the compounds.

One of the primary therapies used to treat disease is the delivery of bioactive compounds to the affected organism. Bioactive compounds may be delivered systemically or locally by a wide variety of methods. For example, an exogenous source (i.e., produced outside the organism treated) of the bioactive compound may be provided intermittently by repeated doses. The route of administration may include oral consumption, injection, or tissue absorption via topical compositions, suppositories, inhalants, or the like. Exogenous sources of the bioactive compound may also be provided continuously over a defined time period. For example, delivery systems such as pumps, time-released compositions, or the like may be implanted into the organism on a semi-permanent basis for the administration of bioactive compounds (e.g., insulin, estrogen, progesterone, etc.).

The delivery of bioactive compounds from an endogenous source (i.e., produced within the organism treated) has also been attempted. Traditionally, this was accomplished by transplanting, from another organism, an organ or tissue whose normal physiological function was the production of the bioactive compound (e.g., liver transplantation, kidney transplantation, or the like). More recently, endogenous production by cells of the affected organism has been accomplished by inserting into the cells a DNA sequence which mediates the production of the bioactive compound. Commonly known as gene therapy, this method includes inserting the DNA sequence into the cells of the organism in vivo. The DNA sequence persists either transiently or permanently as an extra-chromosomal vector (e.g., when inserted by adenovirus infection or by direct injection of a plasmid) or integrates into the host cell genome (e.g., when inserted by retrovirus infection). Alternatively, the DNA sequence may be inserted into cells of the host tissue or in another organism in vitro, and the cells subsequently transplanted into the organism to be treated.

### SUMMARY OF THE INVENTION

In general, the invention features a method of delivering a bioactive compound to an organism. The method includes the steps of growing a plurality of cells in vitro under conditions that allow the formation of an organized tissue, at least a subset of the cells containing a foreign DNA sequence which mediates the production of the bioactive compound, and implanting the cells into the organism, whereby the bioactive compound is produced and delivered to the organism.

In a preferred embodiment of this method, the step of growing may include mixing the cells with a solution of extracellular matrix components to create a suspension, placing the suspension in a vessel having a three-dimensional geometry approximating the in vivo gross morphology of the tissue and having tissue attachments surfaces thereon, allowing the suspension to coalesce, and culturing the coalesced suspension under conditions in which the cells connect to the attachment surfaces and form a tissue having an in vivo-like gross and cellular morphology.

In other preferred embodiments, the DNA sequence encodes the bioactive compound; the DNA sequence encodes a protein which mediates the production of the bioactive compound (for example, by regulating its expression or encoding an intermediate to the bioactive compound); the DNA sequence mediates the production of two bioactive compounds; the tissue includes skeletal muscle; the tissue includes myotubes; the bioactive compound is a growth factor (for example, human growth hormone); the bioactive compound is a bone morphogenetic protein; the bone morphogenetic protein is BMP-6; the organized tissue is implanted into the tissue of origin of at least one of the cells; the cells include a first and a second population of cells, at least a subset of each of the populations containing a foreign DNA sequence which mediates the production of a bioactive compound; the foreign DNA sequence of the first population mediates the production of a bioactive compound different from the foreign DNA sequence of the second population; and the foreign DNA sequence of the first population encodes a bone morphogenetic protein and the foreign DNA sequence of the second population includes a parathyroid hormone.

In other preferred embodiments, the method includes: the step of removing the organized tissue from the organism to terminate delivery of the bioactive compound; following the removal step, the step of culturing the organized tissue in vitro under conditions which preserve its in vivo viability; following the culturing step, the step of reimplanting the organized tissue into the organism to deliver the bioactive compound to the organism; the step of isolating primary cell types of at least one of the cell types of the tissue; and the step of utilizing immortalized cells of at least one of the cell types of the tissue.

In other preferred embodiments of this method, the tissue comprises substantially post-mitotic cells; during the growing step, a force is exerted substantially parallel to a dimension of the tissue; the force is exerted on the individual cells during growth in vitro and on the organized tissue during implantation in vivo; the coalesced suspension exerts a force on the cells substantially parallel to a dimension of the vessel; the cells are aligned substantially parallel to a dimension of the vessel; the vessel is substantially semi-cylindrical in shape; the attachment surfaces are positioned at opposite ends of the vessel; the alignment is mediated by forces exerted by the coalesced suspension; the cells comprise myotubes; the organism is a mammal; and the mammal is a human.

In a related aspect, the invention features an organized tissue producing a bioactive compound, the tissue is produced by the steps of mixing a plurality of cells with a solution of extracellular matrix components to create a suspension, at least a subset of the cells containing a foreign DNA sequence which mediates the production of a bioactive compound; placing the suspension in a vessel having a three dimensional geometry approximating the in vivo gross morphology of the tissue, the vessel having attachment surfaces thereon; allowing the suspension to coalesce; and culturing the coalesced suspension under conditions in which the cells connect to the attachment surfaces and form a tissue having an in vivo-like gross and cellular morphology.

In a related aspect, the invention features an organized tissue producing a bioactive compound. The organized tissue includes a plurality of cells, grown in vitro under conditions that allow the formation of an organized tissue, and a foreign DNA sequence mediating the production of a bioactive compound. The DNA sequence is inserted into at least a subset of the cells. Also included in the invention are organized tissues producing a bioactive compound, the tissue being produced by any of the methods described herein.

In preferred embodiments, the organized tissue is skeletal muscle.

In a related aspect, the invention features an in vitro method for producing a tissue having an in vivo-like gross and cellular morphology. The method includes providing precursor cells of the tissue; mixing the cells with a solution of extracellular matrix components to create a suspension; placing the suspension in a vessel having a three-dimensional geometry approximating the in vivo gross morphology of the tissue, the vessel having tissue attachment surfaces thereon; allowing the suspension to coalesce; and culturing the cells under conditions in which the cells form an organized tissue connected to the attachment surfaces.

In preferred embodiments of this method, the step of providing includes isolating primary cells of at least one of the cell types which make up the tissue or includes utilizing immortalized cells of at least one of the cell types which make up the tissue; the step of providing includes inserting a foreign DNA sequence into at least one of the cells which make up the tissue; the tissue includes substantially post-mitotic cells; the coalesced suspension exerts a force on the cells substantially parallel to a dimension of the vessel; the cells are aligned substantially parallel to a dimension of the vessel; the vessel is substantially semi-cylindrical in shape; and the attachment surfaces are positioned at opposite ends of the vessel.

In other preferred embodiments of this method, the DNA sequence encodes the bioactive compound; the DNA sequence encodes a protein which mediates the production of the bioactive compound; the DNA sequence mediates the production of two bioactive compounds; the bioactive compound is a growth factor; the organized tissue is implanted into the organism, whereby the bioactive compound is produced and delivered to the organism; and the organized tissue is implanted into the tissue of origin of at least one of the cells.

In a related aspect, the invention features an organized tissue produced by the steps of providing precursor cells of the tissue; mixing the cells with a solution of extracellular matrix components to create a suspension; placing the suspension in a vessel having a three-dimensional geometry approximating the in vivo gross morphology of the tissue, the vessel having tissue attachment surfaces thereon; allowing the suspension to coalesce; and culturing the cells under conditions in which the cells form an organized tissue connected to the attachment surfaces. Also included in the invention are organized tissues produced by any of the methods described herein.

In a related aspect, the invention features an apparatus for producing a tissue in vitro having an in vivo-like gross and cellular morphology. The apparatus includes a vessel having a three-dimensional geometry approximating the in vivo gross morphology of the tissue and having tissue attachment surfaces in the vessel.

In preferred embodiments of this aspect of the invention, the apparatus further includes a culture chamber in which the vessel may be submerged; the vessel is substantially semi-cylindrical in shape; the attachment surfaces are coupled to opposite ends of the semi-cylindrical vessel; the coalesced suspension exerts a force on the cells substantially parallel to a dimension of the vessel; and the cells are aligned substantially parallel to a dimension of the vessel.

In a related aspect, the invention features a method of regulating bone formation in an organism. The method includes the steps of growing a plurality of cells in vitro under conditions that allow the formation of an organized tissue, at least a subset of the cells containing a foreign DNA sequence which mediates the production of a bone morphogenetic protein, and implanting the tissue into the organism, whereby the bone morphogenetic protein is produced and delivered to chondroblastic or osteoblastic precursor cells.

In a preferred embodiment of this method, the step of growing may include mixing the cells with a solution of extracellular matrix components to create a suspension; placing the suspension in a vessel having a three-dimensional geometry approximating the in vivo gross morphology of the tissue and having tissue attachments surfaces thereon; allowing the suspension to coalesce; and culturing the coalesced suspension under conditions in which the cells connect to the attachment surfaces and form a tissue having an in vivo-like gross and cellular morphology.

In other preferred embodiments, the DNA sequence encodes the bone morphogenetic protein; the DNA sequence encodes BMP-6; the DNA sequence encodes a protein which mediates the production of the bone morphogenetic protein (for example, by regulating its expression or encoding an intermediate to the bioactive compound); the DNA sequence also mediates the production of another bioactive compound; the tissue includes skeletal muscle; the tissue includes myotubes; the bioactive compound is a growth factor (for example, human growth hormone); the organized tissue is implanted into the tissue of origin of at least one of the cells; the cells include a first and a second population of cells, at least a subset of each of the populations containing a foreign DNA sequence which mediates the production of a bioactive compound; the foreign DNA sequence of the first population mediates the production of a bioactive compound different from the foreign DNA sequence of the second population; and the foreign DNA sequence of the first population encodes a bone morphogenetic protein and the foreign DNA sequence of the second population includes a parathyroid hormone.

In other preferred embodiments, the method includes: the step of removing the organized tissue from the organism to terminate delivery of the bone morphogenetic protein; following the removal step, the step of culturing the organized tissue in vitro under conditions which preserve its in vivo viability; following the culturing step, the step of reimplanting the organized tissue into the organism to deliver the bone morphogenetic protein to the organism; the step of isolating primary cell types of at least one of the cell types of the tissue; and the step of utilizing immortalized cells of at least one of the cell types of the tissue.

In other preferred embodiments of this method, the tissue comprises substantially post-mitotic cells; during the growing step, a force is exerted substantially parallel to a dimension of the tissue; the force is exerted on the individual cells during growth in vitro and on the organized tissue during implantation in vivo; the coalesced suspension exerts a force on the cells substantially parallel to a dimension of the vessel; the cells are aligned substantially parallel to a dimension of the vessel; the vessel is substantially semi-cylindrical in shape; the attachment surfaces are positioned at opposite ends of the vessel; the alignment is mediated by forces exerted by the coalesced suspension; the cells comprise myotubes; the organism is a mammal; and the mammal is a human.

In a related aspect, the invention features a method of providing a bioactive compound to an organism in therapeutic need wherein the method includes the steps of implanting into an organism an organized tissue having an in vivo-like gross and cellular morphology and comprising substantially post-mitotic cells, wherein at least a subset of cells of the organized tissue contain a foreign DNA sequence which mediates the production of a bioactive compound, wherein the bioactive compound is produced in an organism in a therapeutically effective amount.

In a related aspect, the invention features a method of providing a bioactive compound to an organism in therapeutic need wherein the method includes the steps of implanting into an organism an organized tissue comprising substantially post-mitotic cells and having a three-dimensional cellular organization that is retained upon implantation of the tissue into an organism, wherein at least a subset of cells of the organized tissue contain a foreign DNA sequence which mediates the production of a bioactive compound, wherein the bioactive compound is produced in an organism in a therapeutically effective amount.

In a related aspect, the invention features a method of treating a disease in an organism wherein the method includes the steps of implanting into an organism an organized tissue having an in vivo-like gross and cellular morphology and comprising substantially post-mitotic cells, wherein at least a subset of cells of the organized tissue contain a foreign DNA sequence which mediates the production of a bioactive compound, wherein the bioactive compound is produced in an organism in a therapeutically effective amount.

In a preferred embodiment of this method the disease is any one of a blood disorder, a bone or joint disorder, cancer, a cardiovascular disorder, an endocrine disorder, an immune disorder, an infectious disease, a wasting disorder, a neurological disorder or a skin disorder.

In a related aspect, the invention features a method of treating a disease in an organism wherein the method includes the steps of implanting into an organism an organized tissue comprising substantially post-mitotic cells and having a three-dimensional cellular organization that is retained upon implantation of the tissue into an organism, wherein at least a subset of cells of the organized tissue contain a foreign DNA sequence which mediates the production of a bioactive compound, wherein the bioactive compound is produced in an organism in a therapeutically effective amount.

In a preferred embodiment of this method the disease is any one of a blood disorder, a bone or joint disorder, cancer, a cardiovascular disorder, an endocrine disorder, an immune disorder, an infectious disease, a wasting disorder, a neurological disorder or a skin disorder.

As used herein, by a “bioactive compound” is meant a compound which influences the biological structure, function, or activity of a cell or tissue of a living organism.

By “bone morphogenetic protein” is meant an extracellular osteogenic-stimulating molecule belonging to the TGF-β superfamily. Bone morphogenetic proteins (“BMP”) include a large number of proteins, for example, BMP-2,-3,-4,-5,-6,-7,-11, and -12. Bone morphogenetic proteins control the cellular events associated with bone and cartilage formation and repair (e.g., cellular growth, proliferation, and differentiation). For example, bone morphogenetic proteins alter the differentiation pathway of mesenchymal cells towards the chondroblastic or osteoblastic lineage.

By “organized tissue” or “organoid” is meant a tissue formed in vitro from a collection of cells having a cellular organization and gross morphology similar to that of the tissue of origin for at least a subset of the cells in the collection. An organized tissue or organoid may include a mixture of different cells, for example, muscle (including but not limited to striated muscle, which includes both skeletal and cardiac muscle tissue), fibroblast, and nerve cells, but must exhibit the in vivo cellular organization and gross morphology that is characteristic of a given tissue including at least one of those cells, for example, the organization and morphology of muscle tissue may include parallel arrays of striated muscle tissue.

By “in vivo-like gross and cellular morphology” is meant a three-dimensional shape and cellular organization substantially similar to that of the tissue in vivo.

By “extracellular matrix components” is meant compounds, whether natural or synthetic compounds, which function as substrates for cell attachment and growth. Examples of extracellular matrix components include, without limitation, collagen, laminin, fibronectin, vitronectin, elastin, glycosaminoglycans, proteoglycans, and combinations of some or all of these components (e.g., Matrigel™, Collaborative Research, Catalog No. 40234).

By “tissue attachment surfaces” is meant surfaces having a texture, charge or coating to which cells may adhere in vitro. Examples of attachment surfaces include, without limitation, stainless steel wire, VELCRO™, suturing material, native tendon, covalently modified plastics (e.g., RGD complex), and silicon rubber tubing having a textured surface.

By “foreign DNA sequence” is meant a DNA sequence which differs from that of the wild type genomic DNA of the organism and may be extra-chromosomal, integrated into the chromosome, or the result of a mutation in the genomic DNA sequence.

By “substantially post-mitotic cells” is meant an organoid in which at least 50% of the cells containing a foreign DNA sequence are non-proliferative. Preferably, organoids including substantially post-mitotic cells are those in which at least 80% of the cells containing a foreign DNA sequence are non-proliferative. More preferably, organoids including substantially post-mitotic cells are those in which at least 90% of the cells containing a foreign DNA sequence are non-proliferative. Most preferably, organoids including substantially post-mitotic cells are those in which 99% of the cells containing a foreign DNA sequence are non-proliferative. Cells of an organoid retaining proliferative capacity may include cells of any of the types included in the tissue. For example, in striated muscle organoids such as skeletal muscle organoids, the proliferative cells may include muscle stem cells (i.e., satellite cells) and fibroblasts.

The invention provides a number of advantages. For example, implantation of an organized tissue produced in vitro provides quantifiable, reproducible, and localized delivery of bioactive compounds to an organism. Prior to implantation, the production of bioactive compounds by the organized tissue may be measured and quantified per unit time, per unit mass, or relative to any other physiologically-relevant parameter. In addition, the capability of an organized tissue to sustain production of bioactive compounds can be assessed by culturing for extended periods and assaying of compound production with time.

Moreover, because the organized tissue is implanted at a defined anatomical location as a discrete collection of cells, it may be distinguished from host tissues, removed post-implantation from the organism, and reimplanted into the organism at the same or a different location at the time of removal or following an interim period of culturing in vitro. This feature facilitates transient or localized delivery of the bioactive compound. Restriction of the cells producing bioactive compounds to particular anatomical sites also enhances the controlled delivery of bioactive compounds, especially where the organized tissue functions as a paracrine organ. The efficiency of delivery of a bioactive compound (i.e., the amount of the bioactive compound delivered to obtain a desired serum concentration) is also enhanced as compared to direct subcutaneous injection. Likewise, the efficiency of implanting post-mitotic cells containing a foreign DNA sequence into an organism (i.e., the number of cells in a post-mitotic state as a percentage of the initial number of cells containing the foreign DNA sequence) is enhanced by organoid implantation as compared to the implantation of individual mitotic cells. For example, skeletal muscle organoids produced in vitro include post-mitotic myofibers representing greater than 70% of the initial myoblasts containing a foreign DNA sequence, whereas direct implantation of the myoblasts results in post-mitotic myofibers representing less than 1% of the initial cells.

In addition, because substantially all of the implanted cells are fully differentiated, migration of these cells to other anatomical sites is reduced. Moreover, implantation of post-mitotic, non-migratory myofibers containing a foreign DNA reduces the possibility of cell transformation and tumor formation. The implantation of an organized tissue may even enhance the functional and structural characteristics of the host tissue.

Furthermore, because the method of producing a tissue having an in vivo-like gross and cellular morphology may be achieved without the application of external forces by mechanical devices, the apparatus for producing such a tissue is readily adaptable to standard cell and tissue culture systems. The apparatus and method may also be used to produce bone, cartilage, tendon, and cardiac tissues as these tissues include cell types which organize in response to external forces. In addition, the apparatus includes widely available, easily assembled and relatively inexpensive components.

Other advantages and features of the invention will be apparent from the detailed description, and from the claims.

### BRIEF DESCRIPTION OF THE DRAWINGS

FIG. 1 is a diagram of a vessel for growing skeletal muscle tissue which will have an in vivo-like gross and cellular morphology.

FIG. 2 is a bar graph showing results of a comparison of the hematocrits in control animals and animals implanted with EPO-secreting organoids, preimplantation and 7 and 14 days after implantation.

FIG. 3 is a bar graph showing the amount of DNA in a fibroblast organoid at various times in culture.

FIG. 4 is a bar graph showing the amount of IGF-1 secretion from fibroblast organoids in vitro and 1 and 7 days in vivo after implantation.

FIG. 5 is a bar graph showing increased animal size following implantation of IGF-1 secreting fibroblasts.

FIG. 6 is a flow chart of the process of skeletal muscle growth and regeneration.

FIG. 7 is a photograph of skeletal muscle organoids formed in vitro from rhGH-secreting C2C12 cells 48 hours postplating. Top gel has detached and contracted.

FIG. 8 is a micrograph of a section of a skeletal muscle organoid grown in vitro from rhGH-secreting C2C 12 cells which has been stained for sarcomeric tropomyosin.

FIG. 9 is a micrograph of a section of a skeletal muscle organoid grown in vitro from rhGH-secreting C2C12 cells which has been stained for sarcomeric tropomyosin.

FIG. 10(A) illustrates bioartificial organoids engineered from C2C12 myoblasts (C2-organoid) and stained with an antibody to sarcomeric tropomyosin to show the organized muscle fibers. Inset in (A) shows an unstained organoid approximately 30 mm in length; bar equals 0.25 mm and 0.05 mm in inset.

FIG. 10(B) illustrates organoids engineered from primary neonatal rat myoblasts (R-organoid) and stained with an antibody to sarcomeric tropomyosin to show the organized muscle fibers.

FIG. 10(C) is a schematic illustration of retroviral expression constructs which have been used to transduce primary Fisher 344 myoblasts and engineered into R-organoids expressing physiological levels of rhGH.

FIG. 10(D) is a graph showing physiological levels of rhGH produced by R-organoids transduced with the rhGH construct shown in FIG. 10(C).

FIG. 11 is a flow chart comparing myoblast and myofiber gene therapy methods.

FIG. 12A-12F are graphs of rhGH serum levels in mice following skeletal muscle organoid implantation.

FIG. 13A-13B are graphs of the effects of cytosine arabinoside on rhGH-secreting C2C12 proliferating myoblasts and post-mitotic myofibers.

FIG. 14A-14C are photographs of a skeletal muscle organoid grown in vitro from rhGH-secreting C2C 12 cells, implanted in vivo, and subsequently removed and further cultured in vitro.

FIG. 15 is a graph of physiological levels of rhGH produced from primary adult rat myofibers transduced with replication defective retroviral vectors.

FIG. 16(A) is a polyacrylamide gel (left) of equal amounts of urine from C3HeB/FeJ mice implanted at Day 0 with either non-rhGH secreting (control) or rhGH-secreting C2-organoids (hGH); the arrow indicates the position of the 20 kD GH-sensitive liver protein MUP (major urinary protein).

FIG. 16(B) is a bar graph showing results of a comparison of MUP levels in control and rhGH-secreting C2-organoids (hGH) at three weeks after implantation.

FIG. 17 contains bar graphs showing results of attenuation of hindlimb unloading-induced skeletal muscle atrophy with rhGH secreting C2-organoids.(A) and (B) are data for the plantaris muscle while (C) is data for the soleus muscle. Each value is the mean ± SE of 3 to 6 animals and statistical analyses by unpaired t-tests.

FIG. 18 contains bar graphs showing results of attenuation of hindlimb unloading-induced skeletal muscle atrophy in the plantaris but not the soleus muscle with daily rhGH injections.

FIG. 19A-19C are Northern blots of rhBMP-6 MRNA levels in C2C12 cells retrovirally-transduced with a rhBMP-6 gene.

FIG. 20 is a graph of alkaline phosphatase activity in controls and C2C12 cells retrovirally-transduced with a rhBMP-6 gene.

FIG. 21A and 21B are micrographs of C2C12 cells retrovirally-transduced with a rhBMP-6 gene which have been stained for sarcomeric tropomyosin.

FIG. 22 are photographs of cross-sections of R-organoids implanted in adult Fisher 344 rats stained for sarcomeric tropomyosin. Long arrows indicate the surface of the implanted R-organoids, while shorter arrows indicate internal myofibers. (A) and (B) are 7 days postimplantation. Magnification is approximately 12× in (A) and 120× in (B), (C) and (D).

FIG. 23 is a photograph of bioartificial muscles (organoids) engineered from human adult myoblasts stained with an antibody to sarcomeric tropomyosin to show the organized muscle fibers.

FIG. 24(A) is a graph of in vivo rhGH serum levels from rhGH levels secreted in vitro from C2-organoids engineered to contain different numbers of rhGH-secreting myofibers and one organoid per animal was implanted.

FIG. 24(B) is a graph of in vivo rhGH serum levels from rhGH levels secreted ill vitro where the number of C2-organoids implanted per animal was varied from one to four.

### DETAILED DESCRIPTION

I. In Vitro Production of Tissues Having In Vivo-like Gross and Cellular Morphology

Organized tissues having in vivo-like gross and cellular morphology may be produced in vitro from the individual cells of a tissue of interest. As a first step in this process, disaggregated or partially disaggregated cells are mixed with a solution of extracellular matrix components to create a suspension. This suspension is then placed in a vessel having a three dimensional geometry which approximates the in vivo gross morphology of the tissue and includes tissue attachment surfaces coupled to the vessel. The cells and extracellular matrix components are then allowed to coalesce or gel within the vessel, and the vessel is placed within a culture chamber and surrounded with media under conditions in which the cells are allowed to form an organized tissue connected to the attachment surfaces.

Although this method is compatible with the in vitro production of a wide variety of tissues, it is particularly suitable for tissues in which at least a subset of the individual cells are exposed to and impacted by mechanical forces during tissue development, remodeling or normal physiologic function. Examples of such tissues include muscle, bone, skin, nerve, tendon, cartilage, connective tissue, endothelial tissue, epithelial tissue, and lung. More specific examples include skeletal and cardiac (i.e., striated), and smooth muscle, stratified or lamellar bone, and hyaline cartilage. This method is also compatible with the in vitro production of adipose tissue, and tissues comprising either mesenchymal stem cells, bone marrow derived cells, bone marrow stromal cells and neural connective tissue. Organoids comprising primary skeletal myoblasts have been produced and can secrete recombinant human growth hormone. Organoids comprising human fibroblasts have been produced and can secrete recombinant human growth hormone and IGF-1. Organoids comprising rat bone cells have been produced. Where the tissue includes a plurality of cell types, the different types of cells may be obtained from the same or different organisms, the same or different donors, and the same or different tissues. Moreover, the cells may be primary cells or immortalized cells. Furthermore, all or some of the cells of the tissue may contain a foreign DNA sequence which mediates the production of a bioactive compound (as described herein).

The composition of the solution of extracellular matrix components will vary according to the tissue produced. Representative extracellular matrix components include, but are not limited to, collagen, laminin, fibronectin, vitronectin, elastin, glycosaminoglycans, proteoglycans, and combinations of some or all of these components (e.g., Matrigel™, Collaborative Research, Catalog No. 40234). In tissues containing cell types which are responsive to mechanical forces, the solution of extracellular matrix components preferably gels or coalesces such that the cells are exposed to forces associated with the internal tension in the gel.

Culture conditions will also vary according to the tissue produced. Methods for culturing cells are well known in the art and are described, for example, in Skeletal Cell Culture: A PracticaLApproach, (R. I. Fveshney, ed. IRL Press, 1986). In general, the vessel containing a coalesced suspension of cells and extracellular matrix components is placed in a standard culture chamber (e.g., wells, dishes, or the like), and the chamber is then filled with culture medium until the vessel is submerged. The composition of the culture medium is varied, for example, according to the tissue produced, the necessity of controlling the proliferation or differentiation of some or all of the cells in the tissue, the length of the culture period and the requirement for particular constituents to mediate the production of a particular bioactive compound. The culture vessel may be constructed from a variety of materials in a variety of shapes as described below.

An apparatus for producing a tissue in vitro having an in vivo-like gross and cellular morphology includes a vessel having a three dimensional geometry which approximates the in vivo gross morphology of the tissue. The apparatus also includes tissue attachment surfaces coupled to the vessel. Such a vessel may be constructed from a variety of materials which are compatible with the culturing of cells and tissues (e.g., capable of being sterilized and compatible with a particular solution of extracellular matrix components) and which are formable into three dimensional shapes approximating the in vivo gross morphology of a tissue of interest. The tissue attachment surfaces (e.g., stainless steel mesh, VELCRO™, or the like) are coupled to the vessel and positioned such that as the tissue forms in vitro the cells may adhere to and align between the attachment surfaces. The tissue attachment surfaces may be constructed from a variety of materials which are compatible with the culturing of cells and tissues (e.g., capable of being sterilized, or having an appropriate surface charge, texture, or coating for cell adherence).

The tissue attachment surfaces may be coupled in a variety of ways to an interior or exterior surface of the vessel. Alternatively, the tissue attachment surfaces may be coupled to the culture chamber such that they are positioned adjacent the vessel and accessible by the cells during tissue formation. In addition to serving as points of adherence, in certain tissue types (e.g., muscle, bone, nerve, cartilage), the attachment surfaces allow for the development of tension by the tissue between opposing attachment surfaces. Moreover, where it is desirable to maintain this tension in vivo, the tissue attachment surfaces may be implanted into an organism along with the tissue (see further discussion in Section II.).

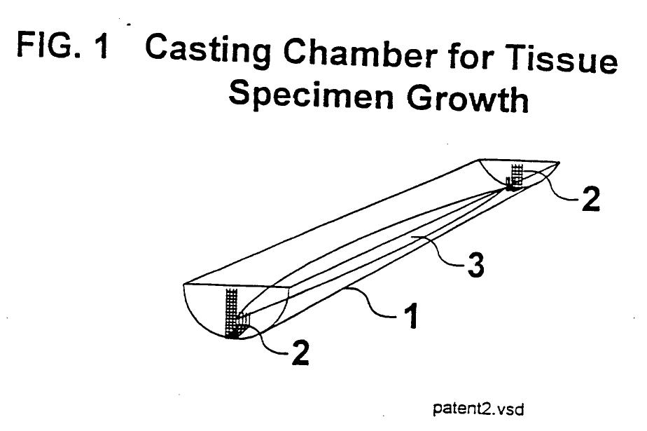

One vessel according to the invention is shown in FIG. 1. This vessel 1, which is suitable for the in vitro production of a skeletal muscle organoid 3, has a substantially semi-cylindrical shape and tissue attachment surfaces 2 coupled to an interior surface of the vessel.

II. Delivery of Bioactive Compounds

Bioactive compounds may be delivered to an organism by growing individual cells in vitro under conditions that result in the formation of an organized tissue producing the bioactive compound and subsequently implanting the organized tissue into the organism (see Section I. for detailed description of organized tissue production). Production of the bioactive compound by the organized tissue is mediated by a foreign DNA sequence present in at least a subset of the cells which make up the implanted tissue.

A variety of bioactive compounds may be delivered by this method, and they may function through intracellular (i.e., within the cells of the organized tissue or organoid), endocrine, autocrine, or paracrine mechanisms. Moreover, the organoid may deliver multiple bioactive compounds either simultaneously or sequentially (e.g., one bioactive compound mediates the delivery of another). Liberation of the bioactive compound from the cells of the organoid may occur by either passive or active processes (e.g., diffusion or secretion).

For example, the bioactive compound may be a hormone, growth factor, or the like which is produced and liberated by the cells of the organoid to act locally or systemically on host tissues. Alternatively, the bioactive compound may function within the cells or on the surface of the cells of the organoid to enhance the uptake or metabolism of compounds from the host tissue or circulation (e.g., lactic acid, low density lipoprotein). Where the organoid serves as a functional and structural adjunct to the host tissue, delivery of growth factors by autocrine or paracrine mechanisms may enhance the integration of the organoid into host tissues. Similarly, where multiple bioactive compounds are produced by the organoid, autocrine delivery of one of the bioactive compounds may be used to regulate the production of one or more of the other bioactive compounds.

The organoid may be implanted by standard laboratory or surgical techniques at a desired anatomical location within the organism. For example, the organoid may be implanted in the same or a different tissue from the tissue of origin of at least one of the individual cells. The location of implantation depends, in part, upon the method of delivery and the identity of the particular bioactive compound to be delivered. For example, an organoid acting as an endocrine organ may be implanted in or adjacent a highly vascularized host tissue. Alternatively, an organoid acting as a paracrine organ is preferably implanted in or adjacent to the host tissue to which the bioactive compound is to be delivered.

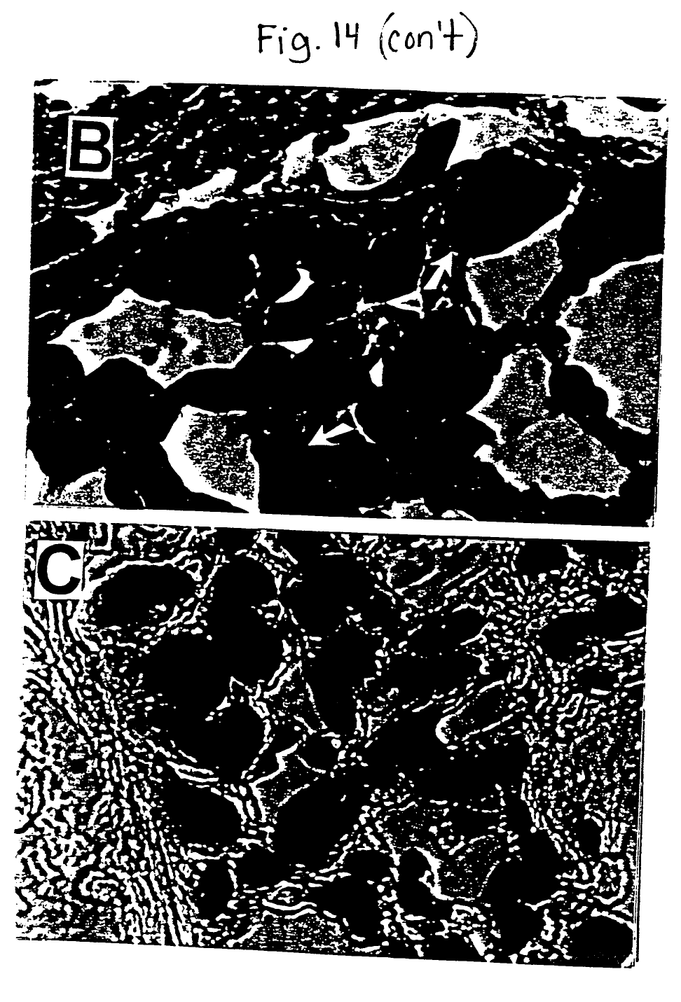

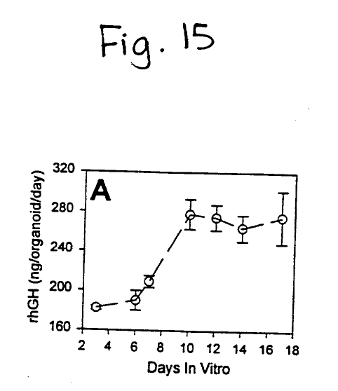

The organoid may be implanted by attachment to a host tissue or as a free floating tissue. In addition, attached organoids may be implanted with or without the tissue attachment surfaces used for in vitro tissue formation. Tissues responsive to mechanical forces are preferably implanted by attaching directly to the host tissue or by implanting the organoid coupled to the attachment surfaces so that the organoid is exposed to mechanical forces in vivo. For example, skeletal muscle organoids are preferably implanted by attachment to the host tissue under tension along a longitudinal axis of the organoid. Moreover, the organoids may be permanently or temporarily implanted. Permanent implantation may be preferred, for example, where the organoid produces a bioactive compound which corrects a systemic metabolic error (e.g., delivery of insulin to treat diabetes), whereas temporary implantation may be preferred where only transient delivery of a bioactive compound is desired (e.g., delivery of a growth factor to enhance wound healing). Furthermore, because organoids may be implanted, removed, and maintained in vitro (see FIG. 14A and discussion below), bioactive compounds may be delivered intermittently to the same or a different location in the organism. For example, a skeletal muscle organoid produced from the cells of a human patient (e.g., an autograft) may be implanted at a first anatomical location for a defined period and subsequently implanted at a second location at or after the time of removal.

At least some of the cells of the organoid contain a foreign DNA sequence. The foreign DNA sequence may be extra-chromosomal, integrated into the genomic DNA of the organoid cell, or may result from a mutation in the genomic DNA of the organoid cell. In addition, the cells of the organoid may contain multiple foreign DNA sequences. Moreover, the different cells of the organoid may contain different foreign DNA sequences. For example, in one embodiment, a skeletal muscle organoid may include myofibers containing a first foreign DNA sequence and fibroblasts containing a second foreign DNA sequence. Alternatively, the skeletal muscle organoid could include myoblasts from different cell lines, each cell line expressing a foreign DNA sequence encoding a different bioactive compound. These “mosaic” organoids allow the combined and/or synergistic effects of particular bioactive compounds to be exploited. For example, myoblasts expressing growth hormone may be combined with myoblasts expressing an insulin-like growth factor to produce organoids useful in stimulating muscle growth/regeneration. Similarly, myoblasts expressing a bone morphogenetic protein may be combined with myoblasts expressing a parathyroid hormone to produce organoids useful in stimulating bone and cartilage growth/regeneration.

In a preferred embodiment, the foreign DNA sequence encodes a protein which is the bioactive compound. The protein is produced by the cells and liberated from the organoid. Alternatively, the DNA sequence may encode an enzyme which mediates the production of a bioactive compound or a cell surface protein which enhances the uptake and metabolism of compounds from the host tissue or circulation (e.g., lactic acid or low density lipoproteins). The DNA sequence may also encode a DNA binding protein which regulates the transcription of the sequence encoding a bioactive compound or an anti-sense RNA which mediates translation of the mRNA for the bioactive compound. The DNA sequence may also bind trans-acting factors such that the transcription of the sequence (i.e., foreign or native) encoding the bioactive compound is enhanced (e.g., by disinhibition). Furthermore, the foreign DNA sequence may be a cis-acting control element such as a promoter or an enhancer coupled to a native or foreign coding sequence for the bioactive compound or for an enzyme which mediates the production of the bioactive compound. Thus, the foreign DNA sequence may be expressible in the cell type into which it is introduced and may encode a protein which is synthesized and which may be secreted by such cells. Alternatively, the foreign DNA sequence may be an element that regulates an expressible sequence in the cell.

III. Treatment of a Disease Bioactive Compounds live by Organized Tissue

The invention provides a method of treating a disease in an organism comprising delivering a bioactive compound to an organism by an organized tissue construct. An organized tissue comprising cells that have been genetically engineered to synthesize and secrete a therapeutically effective amount of a bioactive compound will be implanted into an organism. By “therapeutically effective amount” is meant capable of attenuating the clinical symptoms of a disease or a clinical deficiency associated with a disease in an organism by at least 5-10%, preferably 20-30% and more preferably 35-100%, as compared to an untreated organism. The method of disease treatment according to the invention, is suitable for treating diseases including but not limited to blood disorders, bone and joint disorders, cancer, cardiovascular disorders, endocrine disorders, immune disorders, infectious diseases, wasting disorders, neurological disorders and skin disorders.

### EXEMPLIFICATION

Described below are examples of embodiments of the invention in which a gene of interest (e.g., encoding a protein of interest (or bioactive compound) rhGH, rhBMP, or rhIGF) is introduced into cells (e.g. myoblasts or fibroblasts, primary neonatal rat skeletal myoblasts or fetal human myoblasts) according to the invention. The cells containing the gene of interest are then manipulated and/or permitted to form organoids according to the invention, wherein the organoids produce the protein of interest. The protein-producing organoids are implanted into a mammal and production of the bioactive compound in therapy is demonstrated.

The examples herein below demonstrate the making and using of an bioactive compound-producing organoid to treat a disease according to the invention.

A. Blood Disorders

The invention provides methods of treating blood disorders, including anemia, hemophilia, thrombocytopenia and neutropenia.

Several blood disorders have been treated successfully by the delivery of recombinant human proteins. These disorders include hemophilia, which has been treated by delivery of factor IX (Yao, et al., 1992, Proc. Natl. Acad. Sci. 89, 3357-3361), a plasma glycoprotein essential for blood coagulation, and neutropenia, which has been treated with granulocyte colony stimulating factor (Dale et al., 1993, Blood 81, 2496-2502) which promotes growth, differentiation and functional activity of neutrophils. Anemia has been successfully treated with erythropoietin (EPO) (Hamamori et al., 1994, Hum. Gene. Ther. 5, 1349-1356), the primary regulator of mammalian red blood cell production.

Hemophilia

Hemophilia is an X chromosome-linked recessive bleeding disorder resulting from decreased levels of either factor VIII, factor IX or factor XI (all of which are needed for normal blood coagulation) caused by a genetic abnormality. Hemophiliacs are at risk for bleeding after dental work, surgery, and trauma, and may also suffer internal bleeding with no apparent cause. The most common type of hemophilia (hemophilia A) is a disorder of the intrinsic pathway for the formation of thrombin resulting from a reduction in the coagulant titer of antihemophilic factor (factor VIII:C). Antihemophilic factor is a component of the factor VIII/vWF complex that is regulated by a variety of factors including exercise and hormones; the amino acid sequences necessary for blood coagulation are contained within factor VIII:C.

Hemophilia affects only males who, in turn, pass the abnormal gene onto their daughters, all of whom are carriers. Although women who carry the gene are typically asymptomatic, female carriers can frequently be detected due to the presence of a decreased concentration of factor VIII:C in the plasma, as compared to vWF (Berne and Levy et al., supra). Many individuals with hemophilia die early in life as a result of severe bleeding. However, hemophilia can be treated by transfusion with normal plasma thereby supplying the missing clotting factors and allowing clotting to occur normally on a temporary basis. Although treatment with purified clotting factor (e.g. factor VIII:C) can be used prophylactically to prevent episodes of bleeding (Berne and Levy et al., supra, Guyton, 1985, Anatomy and Physiology, Saunders College Publishing, Philadelphia) because the infused clotting factor remains active for only a short time, serious bleeds may require repeated infusions to stop the bleeding. Often people with severe hemophilia will be treated with prophylactic clotting factor infisions on a regular basis to avoid bleeding episodes.

Treatment of hemophilia by delivery of recombinant human clotting factors would avoid the risk of contamination by human blood-borne viruses, as well as the necessity for frequent infusion treatments. Recently animal models have been developed for the delivery of recombinant human clotting factors. Using a mouse model for severe hemophilia A, donor bone marrow cells were genetically modified to secrete recombinant human factor VIII (GeneBank Accession #119767) and transplanted into hemophiliac mouse recipients (Evans et al., 1998, Proc. Natl. Acad. Sci. USA, 95: 5734-5739). In a second model, C2C12 myoblasts were genetically modified to secrete biologically active factor IX (GeneBank Accession #439774) and injected into the leg muscles of C3H mice, resulting in factor IX expression in the serum (Yao et al., Proc. Natl. Acad. Sci. USA, 89: 3357-3361).

Neutropenia

Neutropenia, a deficiency in circulating neutrophils, leads to a susceptibility to recurrent and often life-threatening infections. Types of neutropenia include chronic congenital, and cyclic, the latter being characterized by regular oscillations in blood neutrophil counts. Neutropenic individuals generally are asymptomatic until the occurrence of an infection. If the neutrophil count decreases to less than 1000 cells per μl, there can be an increase in the risk of infection. A neutrophil count of less than 500 cells per μl can be life threatening. Neutropenia can be caused by a variety of factors including decreased production in the bone marrow, increased destruction of neutrophils in the periphery, or an increase in the rate of neutrophil loss to the tissues. A decrease in neutrophil production can result from a particular disease (e.g. aplastic anemia, or leukemia) or from suppression by a toxic drug or irradiation. Cancer chemotherapy, which kills neutrophils in the bone marrow, is also a cause of neutropenia, and patients with advanced HIV infection frequently have severe neutropenia.

Treatment of neutropenia includes antibiotics to fight infections, and more recently, the injection of G-CSF or GM-CSF to promote the growth, differentiation, and functional activity of cells of the neutrophil lineage (Andreoli et al., 1997, Cecil Essentials of Medicine, Fourth Edition, W.B. Saunders Company, Philadelphia and Berkow et al., editors, 1997, The Merk Manual of Medical Information, Merck Research Laboratories, New Jersey). Recombinant human G-CSF injected into neutropenic patients has been shown to increase neutrophil counts by about 16-fold (Dale et al., 1993, Blood, 81: 2496-2502). In an animal model, primary myoblasts isolated from neonatal Fisher rats were genetically engineered to secrete the human G-CSF gene and injected into the gastrocnemius muscle of adult rats (Bonham et al., 1996, Hum. Gene Ther., 7:1423-1429). Absolute neutrophil counts of rats receiving the transduced myoblasts were significantly increased up to 15 fold following transplantation, while rats implanted with control myoblasts showed no increase in neutrophil counts.

Anemia

Anemia refers to a decrease in the circulating mass of red blood cells (erythrocytes) resulting from decreased production, premature destruction or loss due to hemorrhage. Furthermore, anemia is a symptom of end-stage renal failure. A decrease in erythrocyte synthesis can result from i. hypocellularity of the bone marrow, ii. replacement of the bone marrow by tumor tissue, iii. suppression of hematopoiesis (e.g. during renal failure, or from a vitamin B 12 or folic acid deficiency) or iv. from a deficiency in iron necessary for the formation of heme. A number of factors including hereditary defects in the red blood cell outer membrane, or direct chemical, physical or immunological injury can cause premature destruction of erythrocytes. The most common form of anemia in Western countries is iron-deficiency anemia resulting from either blood loss or the use of iron by the fetus during pregnancy (Berne and Levy eds., 1993, Physiology, Mosby Year Book, St. Louis).

The pathogenesis of a particular form of anemia dictates the method of treatment. For 25 example, iron-deficiency anemia may be treated with iron, pernicious anemia may be treated with vitamin B12, while other forms of anemia may be treated with either red cell replacement or erythropoietin (Berne and Levy, supra).

Erythropoietin (EPO), a 3OkD glycoprotein that functions as the primary regulator of mammalian red blood cell production, increases erythrocyte production by stimulating the proliferation, and preventing the apoptosis of erythroid precursors. Anemia related to diminished red blood cell production in patients with end-stage renal failure has been successfully treated with direct tri-weekly injections of recombinant human erythropoietin (GeneBank Accession #182198, Evans, 1991, Am. J. Kidney Dis., 18: 62-70). However, this method of treatment is expensive and is not the most physiological delivery procedure. Several animal models have been developed for delivery of sufficient quantities of EPO to sustain therapeutic erythropoiesis. These include a gene transfer system in which mouse myoblasts genetically modified to secrete human EPO are injected into the skeletal muscles of mice (Hamamori et al., 1994, Hum. Gene. Ther., 5:1349-1356), and a system wherein autologous smooth muscle cells engineered to secrete rat EPO are infused into the carotid artery of Fisher rats (Osborne et al., 1995, Proc. Natl. Acad. Sci., USA, 92:8055-8058). In both studies, hematocrits were significantly increased by the delivery of recombinant EPO.

Thrombocytopenia

Thrombocytopenia refers to a deficiency in the numbers of platelets in the circulating blood. Because thrombocytopenia is commonly caused by platelet specific antibodies that attack and destroy platelets it is considered an autoimmune disease. Other less common causes of this disease include poisoning by toxins or drugs. In cancer patients thrombocytopenia is caused by impaired platelet production from the bone marrow resulting from chemotherapy or radiation treatment. Thrombopoietin (TPO, Genbank Accession #235118) is the primary regulator of megakaryocyte and platelet production. Animal models have been developed for TPO knockout mice, which have a 90% reduction in platelet counts (Mutone et al., 1998, Stem Cells, 16:1). Recently, thrombocytopenic patients have been treated with recombinant human interleukin-11 (rhIL-11, Genbank Accession #186273; Neumega, Genetics Institute Inc., Cambridge Mass.), a novel thrombopoietic growth factor (Issacs et al., 1997, J. Clin. Oncol., 3368). The potential exists for the delivery of both thrombopoietin and IL-11 for the treatment of thrombocytopenia from organized tissue constructs.

A common symptomatic manifestation of thrombocytopenia is a large number of minute hemorrhages located in the skin and in the deep tissue that eventually cause purplish discolorations over the surface of the body. These hemorrhages result from an inability of the platelets to stop small bleeding points in the vasculature. Although the hemorrhages can be temporarily inhibited by transfusion with either fresh whole blood or separated platelets, both procedures can be difficult to perform (Guyton et al., supra).

### EXAMPLE 1

Treatment of Anemia with Erythropoietin Delivered from Implanted Organized Tissue Constructs

Organized tissue constructs (organoids) composed of postmitotic fibroblasts and myofibers which were genetically engineered to secrete therapeutic levels of erythropoietin were used to increase hematocrits.

Primary muscle and fibroblast cells were isolated from the thigh muscles of 4 week old C3H mice, and genetically engineered (as described in Bohl et al., 1997, Nature Medicine, 3:299) to secrete mouse EPO under the control of the doxycycline-activated promoter from the vector described in Bohl et al., supra. Cells were expanded in culture until nearly confluent, and organoids were formed by suspending 2×10⁶ cells in a 400 μL solution of collagen (1 .6 mg/ml growth medium): Matrigel™ (6:1) and casting the mixture into silicone rubber molds, 4.8 mm i.d.×30 mm long. Resulting organoids contained a mixed population of postmitotic fibroblasts and fused myofibers, with both cell types aligning parallel to the long axis of the mold and containing constant levels of cellular DNA. Some organoids were stimulated in vitro to secrete EPO by the addition of doxycycline (DOX, 1 μg/ml) to the culture medium. After 4 days, DOX-stimulated organoids secreted 105.5±5.3 U EPO/day, while unstimulated organoids secreted 4.1±0.2 U EPO/day. Organoids produced from cells that were not genetically engineered to contain the EPO gene do not express EPO (data not shown). C3H mice to be implanted with DOX-stimulated organoids were given DOX in their drinking water (200 μg/ml in 5% sucrose) beginning 4 days before implantation. The normal EPO level in these animals prior to implantation was less than 0.05 U/mL serum.

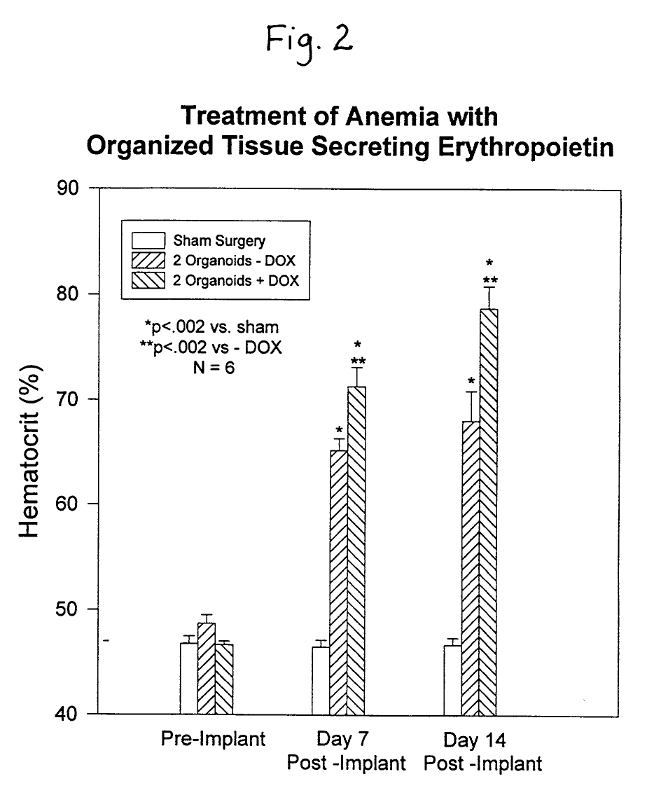

In vitro DOX-stimulated or unstimulated organoids were implanted under tension into 6 week old C3H mice by anesthetizing the mice with metafane, shaving and sterilizing their backs and making a 30 mm incision alone the midline. The skin at the site of the incision was reflected, two organoids were inserted under the skin, and the wound was sutured closed. Hematocrits were measured from tail bleeds 4 days prior to surgery, and on days 7 and 14 after implantation. Sham surgery was performed on a third group of animals. Day 7 and Day 14 hematocrits of mice implanted with DOX-stimulated organoids secreting 105 U EPO/day were significantly increased compared to both sham implanted mice (Day 7: 71.3±0.3 U/mL vs. 46.5±0.6 U/mL, P<0.002; Day 14: 78.7±2.1 U/mL vs. 46.7±0.7 U/mL, p<0.002) and mice implanted with DOX-unstimulated organoids secreting 4.1 U EPO/day (Day 7: 65.2±0.8, P<0.03; Day 14: 68.0±2.9; P<0.02) (FIG. 2).

The method of delivering EPO by organoids offers the advantage of causing a more rapid increase in the hematocrit as compared to other cell-based delivery techniques. The delivery of EPO by organoids stimulated an increase in the hematocrit in one week, while other procedures (Hamamori et al., supra) required three to four weeks to obtain an equivalent increase in the hematocrit. The method of delivering EPO by organoids also offers the advantage of being reversible. The rapid increase in hematocrit stimulated by EPO delivery from organoids offers promise for the long-term rapid treatment of anemia.

Other blood disorders, including hemophilia, neutropenia and thrombocytopenia may also be treated by using organized tissue constructs that are genetically engineered to secrete the relevant molecules required for treatment (described below).

Organoids producing EPO may be tested in an animal model of anemia (e.g. see Hamamori et al., supra or Osborne et al., supra) by implanting one or more organoids producing EPO into the anemic animal and determining the level of EPO and the hematocrit of the treated animal over time.

Several animal models have been developed for delivery of sufficient quantities of EPO to sustain therapeutic erythropoiesis. One of the features of a mouse model of renal failure is that the mouse become anemic (Hamamori et al., supra). A renal failure model was created by a two-step nephrectomy using 7-8 wk-old male nude mice. Under general anesthesia using sterile techniques, the right kidney was exposed through a flank incision and decapsulated, and the upper and lower poles (two thirds of the right kidney) were resected. The remnant right kidney was allowed to recover from swelling for a week, and then the total left kidney was resected. Renal failure was confirmed by the development of both anemia and uremia. (Hamamori et al., supra). The hematocrits of these mice can be increased by using a gene transfer system in which mouse myoblasts genetically modified to secrete human EPO are injected into the skeletal muscles of mice (Hamamori et al., supra).

In a second animal model, hematocrits of Fisher rats were increased following infusion of autologous smooth muscle cells engineered to secrete rat EPO into the carotid artery (Osborne et al., supra). Ecotropic PE501 and amphotropic PA317 retrovirus packaging cell lines, NIH 3T3 thymidine kinase-negative cells, and primary cultures of rat smooth muscle cells were grown in Dulbecco-Vogt-modified Eagle's medium with high glucose (4.5 g/liter) supplemented with 10% fetal bovine scrum in humidified 5% CO₂/95% air at 37° C.

Rat smooth muscle cell cultures were prepared by enzymatic digestion of the aorta from male fisher 344 rats. These cells were characterized by positive staining for muscle cell-specific actins with HHF35 antibody while staining negative for von Willebrand factor, an endothelial cell-specific marker. Early passage smooth muscle cells were exposed to 16-hr virus harvests from PA317-LrEPSN and PA317-LASN amphotropic virus-producing cell lines for a period of 24 hr in the presence of Polybrene (4 μg/ml). Vascular smooth muscle cells infected with LrEPSN and selected in G-418 antibiotic (1 mg/ml) secreted 6.7 milliunits per 24 hr per 10⁵ cells of EPO as determined by an ELISA assay procedure constructed to measure human EPO (R&D Systems). Biological activity of vector-encoded EPO was confirmed by proliferation of a murine erythroleukemia cell line (HCD-57) sensitive to recombinant human EPO. Transduced EPO-secreting smooth muscle cells showed the same growth characteristics as control cells both in vitro and in vivo, indicating the absence of any EPO-mediated autocrine effect.

For cell seeding, rats were anesthetized, and the left carotid artery was temporarily isolated with ligatures and denuded of endothelium by passage of a balloon catheter introduced through an arteriotomy in the external branch. Transduced vascular smooth muscle cells (106 cells in 50 μl of culture medium) were infused over 15 min. into the isolated carotid segment by means of a cannula in the external carotid segment after a brief irrigation with culture medium. The external carotid segment was ligated after removal of the catheter, blood flow was restored, and the wound was closed. Anticoagulated blood samples (100 μl) were obtained from the tail vein, and reticulocyte count was determined by vital staining with brilliant cresyl blue and counting 1000 cells by standard techniques. Hematocrit, hemoglobin, platelet, and white blood cell (WBC) number were measured with a Coulter Counter (Osborne et al., supra). Both studies demonstrate that hematocrits can be significantly increased by the delivery of recombinant EPO.

Anemic human patients may be treated accordingly by implanting one or more EPO-producing organoids and measuring EPO levels, hematocrits, and the alleviation of symptoms of anemia over time.

### EXAMPLE 2

Treatment of Hemophilia with Factor IX Delivered from Implanted Organized Tissue Constructs

Postmitotic organoids genetically engineered to deliver therapeutic levels of recombinant protein clotting factors e.g. factor IX are used to treat hemophilia in C3H mice. Cells (e.g. myoblasts or fibroblasts)are isolated from 4 week old C3H mice, and plated into tissue culture flasks. When the cells are nearly confluent they are harvested and plated at low density in 35 mm diameter tissue culture plates. The low density cultures are transduced with the LIXSN retroviral vector, which contains a 1.4 kilobase human factor IX cDNA under the control of the 5′ long terminal repeat (LTR) (Yao et al., 1991, Proc. Natl. Acad. Sci. USA, 89:8101-8105). Transduction with the viral vector is achieved by incubating the cultures with viral medium supplemented with 8 μg/mL polybrene, centrifuging the plates at 2500 rpm for 30 min, removing the viral medium, and feeding with fresh growth medium. After a total of 5 similar transduction centrifugations over 48 hours, cells are harvested, plated into 10 cm dishes and expanded until confluent. Organoids are produced from transduced cells and control, non-transduced cells as described in Example 1. The amount of human factor IX secreted from the organoids in vitro is quantitiated by an ELISA (Yao et al., supra). It is expected that in vitro transduced cells in organoids will secrete significantly greater amounts of factor IX than non-transduced control organoids.

One to four Factor IX secreting and non-secreting organoids are implanted under tension in 6 week old C3H mice as described in Example 1. In vivo serum levels are measured by ELISA from tail bleeds at varying time points after implantation and it is expected that these levels will be significantly higher than the serum levels of Factor IX in mice implanted with non-transduced control organoids.

Organoids producing Factor IX may be tested in an animal model of hemophilia (e.g. see Evans et al., supra) by implanting one or more organoids producing Factor IX into the animal, determining the level of Factor IX, and measuring blood clotting in the treated animal over time.

Several animal models have been developed for the delivery of clotting factors in the treatment of hemophilia. Donor bone marrow cells that were genetically modified to secrete recombinant human factor VIII have been transplanted into hemophiliac mouse recipients (Evans et al., supra). The murine Factor VIII gene and protein are highly homologous to their human counterparts. Two lines of Factor VIII-knockout mice were generated by Neo gene disruptions in exon 16 or 17 of the murine Factor VIII gene. These mice completely lack plasma Factor VIII activity and do not survive tail biopsies without cautery. Whereas both lines of mice are devoid of Factor VIII light chain antigen in the plasma it is not known whether Factor VIII heavy chain antigen is present. Thus, it is not known whether these mice are immunologically Factor VIII-naive for all Factor VIII epitopes. However, these mice do mount a Factor VIII inhibitor antibody response after repeated i.v. injection of human Factor VIII, in the absence of adjuvant. Factor VIII knockout mice have been derived by serial breeding of a 129SV founder knockout mouse three times with inbred C57BL/6 mice, followed by inbreeding (Evans et al., supra).

In a second animal model, Factor IX expression in the serum has been induced by injecting C2C12 myoblasts, genetically modified to secrete biologically active factor IX, into the leg muscles of C3H mice (Yao et al., supra).

Therapeutic efficacy of treatment of hemophilia according to the invention by implantation of an organized tissue producing factor IX as described herein, is indicated by changes in clinical parameters such as increased blood clotting (e.g. at least 5-10% and preferably 25-100%). Clotting is measured clinically by the activated partial thrombin time test (apTT). Activating agents are added to the plasma initiating a series of reactions which lead to the conversion of fibrinogen to fibrin. Clotting time is recorded as the interval from the appearance of the first fibrin threads after initial activation. The rate of clotting is a measure of the overall coagulant activity (Williams et al., 1983, in Hematology, 3rd edition, p. 1662-1663).

Human hemophilia patients may be treated accordingly by implanting Factor IX-producing organoids and measuring Factor IX levels, blood clotting and the alleviation of symptoms of hemophilia over time

### EXAMPLE 3

Treatment of Neutropenia with Granulocyte Colony-stimulating Factor (G-CSF) Delivered from Implanted Organized Tissue Constructs

Postmitotic organoids genetically engineered to deliver therapeutic levels of recombinant human G-CSF to rats are used to treat neutropenia in murine models. Cells are isolated from the hind limb muscles of newborn Fisher rats, and plated into tissue culture flasks. When the cells are nearly confluent they are harvested and plated at low density in 35 mm diameter tissue culture plates. The low density cultures are transduced with the LghGSN retroviral vector, which contains the human G-CSF gene under transcriptional control of the Moloney murine leukemia virus LTR (Bonham et al., 1996, Human Gene Therapy, 7:1423) as described in Example 2. Organoids are produced from transduced cells and control, non-transduced cells as described in Example 1. The amount of G-CSF secreted from the organoids in vitro is quantitiated by assaying cell supernatants for the ability to support proliferation of a growth factor-dependent myeloblastic cell line, NSF-60 (Shirafuji et al., 1989, Exp. Hematol., 17: 116-119). It is expected that in vitro transduced cells in organoids will secrete significantly greater amounts of G-CSF than non-transduced control organoids.

G-CSF secreting and non-secreting organoids are implanted under tension into adult Fisher rats by anesthetizing by IP injection of 55 mg/kg nembutal, shaving and sterilizing the back, and making a 50 mm incision along the mid-line. The skin at the site of the incision is reflected, one or more organoids are inserted under the skin and the wound is sutured closed. Absolute neutrophil counts are determined from blood samples at various times after implantation by differential analysis of Wright's stained peripheral blood smears. It is expected that there will be a significant increase in the neutrophil count of rats implanted with G-CSF secreting organoids as compared to rats implanted with non G-CSF secreting implanted organoids. The increased neutrophil count in these animals is expected to be adequate for treating neutropenia.

Organoids producing G-CSF may be tested in an animal model useful for studying treatment of neutropenia (e.g. see Bonham et al., supra) by implanting one or more organoids producing G-CSF into the animal and determining the level of GCS-F and the neutrophil count in the treated animal over time.

Several animal models have been developed for delivery of sufficient quantities of G-CSF to cause an increase in neutrophil counts (e.g. see Bonham et al., supra). Following the injection of rat myoblasts genetically engineered to secrete the human G-CSF gene, into the gastrocnemius muscle of adult rats, an increase in the absolute count of neutrophils was observed (Bonham et al., supra).

Primary human myoblasts were isolated from an intercostal muscle biopsy of a 5-year-old female donor. The muscle tissue was minced and dissociated with collagenase D (4 mg/ml; Boehringer Mannheim) in Dulbecco's modified Eagle's medium (DMEM with high glucose (4.5 grams/liter). After vortexing the suspension, the total cell suspension and small fiber fragments were plated onto 10-cm dishes dish coated with type I rat tail collagen (Collaborative Research). Nonadherent debris was removed 48 hr later. After 2-3 weeks growth in DMEM with 10% fetal bovine serum (GIBCO BRL), the cells were harvested and sorted by labeling with the muscle-specific antibody 5.1H11 and a secondary anti-mouse antibody labeled with fluorescein isothiocyanate (FITC). Intact cells were identified and gated on forward/right-angle light scatter. Cells with fluorescence greater than that of cells exposed only to the secondary antibody were collected as 5.1H11-positive myoblasts. Differentiation from myoblasts into myotubes was induced by growing the cells for approximately 72 hr in DMEM supplemented with 1% horse serum.

Primary rat myoblasts were prepared from the hind limb muscles of newborn (3-to 5-day-old) Fisher 344 rats. The muscle tissue was minced and dissociated by trypsin and collagenase treatment, followed by Percoll (Sigma) gradient centrifugation. The cells were grown in DMEM with 10% fetal bovine serum and 1% chick embryo extract (GIBCO BRL) on dishes coated with type 1 rat tail collagen (Collaborative Research). These cultures were shown to be approximately 70% positive for the muscle-specific marker myogenin using the F5D anti-rat myogenin monoclonal antibody and fluorescence analysis. Differentiation in vitro was induced as described above for human myoblasts.

Virus-containing medium was collected from confluent dishes of virus-producing cells, filtered (0.45 μm), and stored at −70° C. until use. Beginning at 48 hr after isolation, myoblasts were infected three times over 3 consecutive days in medium from the PA317 vector-producing cells in the presence of 4 μg/ml Polybrene. The medium was replaced with fresh virus-containing medium supplemented with 1% check embryo extract on each of the 3 days.

At 72 hr prior to transplantation of myoblasts, animals were treated with 0.5 ml of 0.75% Marcaine distributed between the gastrocnemius muscles of both hind legs in several 50- to 100-μl injections. Myoblasts infected with either LghGSN or LgZnSN were trypsinized, washed, and resuspended in serum-free medium (˜10⁸ cells/ml). The cells (10⁸ per animal) were introduced into the Marcaine-treated gastrocnemius muscle by multiple injections into both legs. All animals receiving myoblast transplants were injected daily with 5 mg/kg Cyclosporin A for the duration of the study, beginning 24 hr prior to transplant. Halothane was used to anesthetize the rats prior to all injections (Bonham et al., supra).

Therapeutic efficacy of treatment of neutropenia according to the invention by implantation of an organized tissue producing G-CSF as described herein, is indicated by changes in clinical parameters such as increased neutrophil counts (e.g. at least 5-10% and preferably 25-100%).

Neutropenic human patients may be treated accordingly by implanting G-CSF producing organoids and measuring G-CSF levels, neutrophil numbers and the alleviation of symptoms of neutropenia over time.

B. Bone or Joint Disorders

The invention provides methods of treating bone or joint disorders, including osteoporosis and osteoarthritis.

Osteoarthritis

Osteoarthritis (also known as degenerative arthritis or degenerative joint disease) is an age-related, chronic disorder of the joints that is associated with degeneration of joint cartilage and formation of new bone at the joint surfaces, often causing pain and stiffness. A variety of biological and mechanical factors can result in osteoarthritis. Osteoarthritis can generally be classified as primary (associated with aging) or secondary (associated with a well-defined cause e.g. inflammatory or connective tissue disease).

Numerous pathologic changes including cartilage fibrillation, fissuring, and erosion (leading to bare areas of bone), spur formation at joint margins, and sclerosis and thickening of subchondral bone are associated with osteoarthritis. The major symptoms of osteoarthritis include progressive pain and stiffness in the joints (most typically hips, knees, spine and small joints of the hands and feet). Other symptoms may include cracking of the joint, deformity due to joint enlargement, and limitation of motion.

Methods of treatment of osteoarthritis may include appropriate forms of exercise, supports or braces, physical therapy, surgery and the administration of analgesics or nonsteroidal anti-inflammatory drugs to reduce pain and swelling (Andreoli et al., 1997, supra and Berkow et al., supra). Transforming growth factor beta (TGF-β) has powerful modulatory effects on the skeletal system, enhancing bone formation and decreasing matrix degradation, thus playing a part in the maintenance of bone mass (Boonen et al., 1997, J. Internal Med., 242:285-290). It has been suggested that interleukin-1 receptor antagonist, as well as other recombinant proteins, may be potentially useful for preventing and treating osteoporosis by stimulating bone formation (Evans et al., 1998, Ann. Rheum. Dis., 57:125).

Mice that are aged 7 months and older develop spontaneous osteoarthritic lesions in the mandibular condyle cartilage of the temporomandibular joint, and thereby provide an art-accepted model for studying cartilage loss associated with osteoarthritis (Livne et al., 1985, Arthritis and Rheumatology, 28:1027-1038).

Osteoporosis

Osteoporosis, the most common form of metabolic bone disease, is characterized by a reduction in bone mineral and bone matrix that produces bone that is of a normal composition but is decreased in density and is therefore more likely to fracture. Typically, osteoporosis results from the normal effects of menopause in women, and aging, in both men and women. However, other disorders including glucocorticoid excess, hypogonadism, hyperthyroidism, hyperparathyroidism, vitamin D deficiency, gastrointestinal diseases, bone marrow disorders, immobilization, connective tissue diseases and certain drugs can cause osteoporosis.

In the absence of the occurrence of a fracture, osteoporosis is asymptomatic. Following the occurrence of bone collapse or fracture, bone pain may occur and deformities may develop. The most common types of fractures in patients with osteoporosis are vertebral compression fractures or fractures of the wrist, hip, pelvis or humerus. Osteoporosis can be diagnosed prior to the occurrence of a fracture by a variety of methods that measure bone density. These measurements can also be used to predict the development of certain osteoporotic fractures.

Although presently, established osteoporosis cannot be reversed, methods of early intervention can prevent osteoporosis in most individuals, and later intervention can inhibit the progression of the disease. Methods of treatment of osteoporosis include increasing dietary calcium (calcium can slow but not prevent bone loss in women in the early stages of menopause), estrogen treatment (estrogen replacement therapy prevents bone loss in estrogen deficient women), calcitonin treatment (calcitonin appears to prevent loss of bone in the spine of women in either the early or late stages of menopause without affecting appendicular bone loss), biophosphonates (biophosphonates inhibit resorption of osteoclastic bone) and vitamin D and its metabolites (Andreoli et al., supra and Berkow et al., supra).

Recombinant proteins can be useful for attenuating osteoporosis. Bone morphogenetic protein (BMP) is a family of bioactive factors that stimulate new bone formation in ectopic sites by inducing the differentiation of primitive mesenchymal cells into bone producing cells (Strates et al., 1988, Am. J Med. Sci., 296:266-269). Therefore, recombinant human bone morphogenetic protein (rhBMP) may be useful for the treatment of osteoporosis (Urist et al., 1985, Progress in Clinical and Biological Research, 187:77-96). Growth hormone (GH) has been thought to augment bone turnover, increase bone formation and, to a lesser extent, increase bone resorption (Inzucchi et al., 1994, J. Clinical Endocrinol. Metab., 79: 691-694). GH replacement therapy may be a useful method of treating osteoporosis. Insulin-like growth factor-I (IGF-I) enhances cartilage and bone formation, and decreases matrix degradation, thereby indicating that it is an important stimulator of skeletal growth and is relevant to the maintenance of bone mass (Schmid, 1993, J. Int. Med., 234: 535-542). IGF-I replacement therapy may be useful for treatment of osteoporosis. Platelet-derived growth factor-BB (PDGF-BB) is one of the many systemic factors involved in the bone formation cascade at sites of bone resorption (Watrous et al., 1989, Seminars in Arthritis and Rheumatology, 19: 45-65). Therefore, recombinant human platelet-derived growth factor (rhPDGF-BB) may be useful for stimulating bone formation in the prevention and treatment of osteoporosis (Watrous et al., supra).

Although parathyroid hormone (PTH) had initially been thought to be a catabolic agent to the skeletal system, recent evidence has suggested that PTH exerts a direct inhibitory effect on bone resorption and an indirect stimulatory effect on bone resorption mediated by osteoblasts (Dempster et al., 1993, Endocrine Review, 14:690-709). Therefore, recombinant human parathyroid hormone (rhPTH) may be useful for the treatment of osteoporosis (Reeve, 1996, J. Bone and Mineral Research, 11:440-445).

TGF-β has powerful modulatory effects on the skeletal system, enhancing bone formation and decreasing matrix degradation, thus playing a part in the maintenance of bone mass (Boonen et al., supra). Therefore, recombinant human TGF-β may be a useful drug for stimulating bone formation in the prevention and treatment of osteoporosis (Boonen et al., supra).

Several animal models have been useful for studies of osteoporosis, most notably the ovariectomized (OVX) rat. OVX rats display significantly decreased trabecular bone volume (41%) and decreased mechanical strength of the femoral neck (15.8%) (Peng et al., 1994, Bone, 15:523-532).

### EXAMPLE 4

Treatment of Osteoarthritis with Recombinant Protein from Implanted Organized Tissue Constructs.

Organized tissue constructs (organoids) genetically engineered to produce a recombinant protein (interleukin-1 receptor antagonist, IL-IRA) are used to deliver therapeutic levels of an osteoarthritic animal (Livne et al., supra). The effects of a sustained release of recombinant proteins on cartilage remodeling and mechanical strength in an osteoarthritic animal model is determined and will provide useful information that is directly relevant to treating the human disease.

Cells (e.g. fibroblasts or myoblasts) are isolated from animals and plated separately into T-75 flasks. When the cells are nearly confluent they are harvested and plated at low density in 35 mm diameter tissue culture plates. The low-density cultures are transduced with the MFG retroviral vector, which contains the gene for interleukin-1 receptor antagonist (Evans et al., supra).

Transduced cells are engineered into organoids for each individual animal (i.e. autologous implants) as described in Example 1. In vitro, transduced cells in the organoids are expected to secrete significantly greater amounts of rhIL-IRA than non-transduced control organoids. One or more recombinant protein secreting organoids are implanted under tension in mice as described in Example 1. Organoids are inserted subcutaneously or into the muscle bed. The in vivo level of rhIL-1 RA in the tissue or serum is measured at several time points following implantation in order to demonstrate that there is a significant increase in the levels of rhIL-1RA as compared to animals in which non-rhIL-IRA secreting organoids are implanted.

One may test the therapeutic efficacy of treatment of osteoarthritis by implanting an organoid producing a recombinant protein (e.g.rhIL-1RA) and determining if there is an inhibition (at least 5-10% and preferably 25-100%) in the destruction of joint tissue. Joint tissue breakdown can be measured biochemically by assessing proteoglycan content, acid phosphatase content, and protein and glycosaminoglycan synthesis rates (Ehrlich et al., 1975, J. Bone and Joint Surgery, American, 57:392). Histology, histomorphometry and fluorescence microscopy can also be used to assess articular cartilage pathology (Armstrong et al., 1994, J. Rheumatol., 21:680). This may be tested in an animal model of osteoarthritis (e.g. see Livne et al., supra) comprising mice that have spontaneously developed osteoarthritic lesions.

Spontaneous osteoarthritis is a common phenomenon in the temporomandibular joints of ICR mice, from early neonatal life until they reach senescence. Studies of the light microscopic, ultrastructural, and cytochemical characteristics of the temporomandibular joints of ICR mice have demonstrated that aging of mandibular condylar cartilage was accompanied by decreasing total proteoglycan content and by an unmasking of collagen fibers, with no shift in collagen type. Fibronectin was also commonly present on the articular surface of specimens from old animals. Chondrocytes of aged mice contained an increased number of lysosomes, and their adjacent matrix vesicles reacted positively for acid phosphatase and arylsulfatase, but not for alkaline phosphatase. Such vesicles were also found to be devoid of calcium complexes and, thus, did not appear to be involved in the mineralization process. Similar age-related changes have been described in human mandibular condyles; hence, the male ICR mouse could serve as a useful model for studies of spontaneous osteoarthritis in the human mandibular joint (Livne et al., supra). Human osteoarthritis patients may be treated accordingly by implanting rhIL-1RA-producing organoids and measuring rhIL-1RA levels, joint tissue destruction and the amelioration of the symptoms of osteoarthritis at different time points following implantation.

### EXAMPLE 5

Treatment of Osteoporosis with Recombinant Protein Delivered from Implanted Organized Tissue Constructs.

Organized tissue constructs (organoids) genetically engineered to produce a recombinant protein (e.g. BMP, GH, IGF-1, PTH, PDGF and/or TGF β) are used to deliver therapeutic levels of these proteins to an ovariectomized rat. The effects of sustained release of a recombinant protein on bone remodeling and mechanical strength in an osteoporotic animal model are determined.

Cells (e.g. fibroblasts or myoblasts) are isolated from animals and plated separately into T-75 flasks. When the cells are nearly confluent they are harvested and plated at low density in 35 mm diameter tissue culture plates. The low density cultures are transduced with the MFG retroviral vector containing the gene encoding a recombinant protein (e.g. rhBMP-2, GENBANK Accession #115068; hGH, GENBANK Accession #1311018; rhIGF-I, GENBANK Accession #s32990, 32992; rhPTH, GENBANK Accession #s131547, 2144647; rhPDGF-BB, GENBANK Accession #s494431, 494432, 494433; rhTGF-β, GENBANK Accession #s339558, 339560, 339562, 339564) as described in Example 2.

Transduced cells are engineered into organoids, as described in Example 1. In vitro, transduced cells in the organoids should secrete significantly greater amounts of recombinant protein than non-transduced control organoids. Up to 10 recombinant protein secreting organoids are implanted into the muscle beds of rats under tension, as described in Example 3. The in vivo level of recombinant protein in the tissue or serum is measured at several time points following implantation in order to demonstrate that there is a significant increase in the level of recombinant protein as compared to animals in which non-recombinant protein secreting organoids are implanted. The increase in recombinant protein levels in the experimental animals is significant where a sufficient amount of the protein is produced such that an improvement in the clinical symptoms of osteoporosis is indicated by increased bone volume, density, and/or strength.

Dual-energy x-ray absorptiometry (DXA) estimates the bone mineral content (BMC), which can also produce bone mineral density (BMD) when divided by volume (Rosen et al., 1995, J. Bone and Mineral Res., 10:1352). Quantitative computed tomography can measure bone mass, balance, and dimensions of any important skeletal fraction, i.e. epiphyseal and metaphyseal spongiosas (Genant et al., 1989, Radiology, 170:817). Bending and torque tests on femoral or tibial diaphyses are the usual method for measuring ultimate strength, stiffness, and yield points of whole bones and of bone as a tissue (Turner and Burr, 1983, Bone, 14:595). Histomorphometry can be used to measure cortical thickness, trabecular bone volume, and cross-section area (Recker, 1983, Bone Histomorphometry, Techniques and Interpretation., CRC Press, Boca Raton).

Standardized quantitative increases in the above parameters for determining therapeutic efficacy of an osteoporosis treatment have not yet been specified. It is known in humans that bone loss can be from 30-50% over a 10-40 year period (Adami et al., 1995, Osteoporosis International, 5:75). This has not been extended to quantitative losses in bone mineral content or density, fracture strength, or cortical/medullary cross-sectional areas. Several osteoporosis consensus conferences have defined osteoporosis as an increase in the risk of fracture due to decreased bone mass (Am. J. Med., 94:646). One may test the therapeutic efficacy of treatment of osteoporosis by implanting an organoid producing a recombinant protein (e.g. BMP, GH, IGF-1, PTH, PDGF and/or hTGF-β) and determining if there is an increase in bone volume, density, and/or strength. This may be tested in an animal model of osteoporosis (e.g. see Peng et al., supra) comprising ovariectomized rats that demonstrate decreased trabecular bone volume and decreased mechanical strength of the femoral neck.

For ovariectomy (OVX) experiments, female Sprague-Dawley rats at the age of 12 weeks (243±16 g) were either ovariectomized (n=14) or sham-operated (n=14), and killed 6 weeks after operation. The operations were carried out using a dorsal approach. The ovaries were removed together with the oviducts and a small portion of the uterus (Peng et al., supra).

Human osteoporosis patients may be treated accordingly by implanting recombinant protein-producing organoids, measuring the level of recombinant protein produced by the organoids, determining bone volume, density and strength, and measuring the amelioration of the symptoms of osteoarthritis at different time points following implantation.

C. Cancer

The invention also provides methods of treating cancer.

Cancer is a disease that is characterized by uncontrolled growth of abnormal or cancerous cells, in most instances as a result of an altered genome in a single abnormal cell. The alteration in the genome is caused by a mutation in one or more genes wherein the probability of the occurrence of a mutation is increased by a variety of factors including i. ionizing radiation, ii. exposure to chemical substances known as carcinogens, iii. some viruses, iv. physical irritation, and v. hereditary predisposition. It is thought that a single mutation is insufficient to convert a normal cell into a cancer cell, and that cancer is caused by several independent genetic alterations (Guyton, supra, Alberts et al., 1994, Molecular Biology of the Cell, Garland Publishing, Inc., New York).

Neoplasms including solid tumors such as malignant melanoma, and blood-borne cancers such as leukemia, arise from normal cell populations which have lost the ability to adequately respond to either intracellular or extracellular growth controlling mechanisms. Furthermore, cancer cells are less adherent to each other, as compared to normal cells. As a result, these abnormal cell populations divide at a more rapid rate than their normal cellular counterparts and, in the case of solid tumors, are capable of invading adjacent tissue. Cancerous cells enter the blood stream, migrate to distant sites within the body and eventually colonize secondary organs, a process known as metastasizing. Much of the damage of cancer cells results from the overuse of nutrients by cancer cells (due to the fact that they proliferate indefinitely) as compared to normal cells.

Cancers are classified according to the tissue and cell type from which they are derived and each type of cancer demonstrates characteristics that reflect the cell type of origin. In general, cancers that originate from different cell types are associated with different diseases (Guyton, supra, Alberts et al., supra).

Several therapeutic approaches have been used to slow the progression of dividing tumors. En bloc resection of the primary tumor followed by radiation therapy, chemotherapy or a combination of the two are conventional methods employed to treat the vast majority of tumor types. These modalities, however, can be ineffective and potentially harmful. The site of the tumor, surgical complications such as hemorrhage and the inability to locate tumor masses in a diseased organ can hinder potentially effective operative procedures. In addition, radiotherapy and chemotherapy are associated with ionizing damage of healthy tissue and systemic toxicity respectively.

Alternative approaches to the conventional treatments described above may include the delivery of recombinant molecules which function to either boost the host's immune response to invading metastases or to either directly or indirectly suppress cancerous cell growth. Such molecules may include various cytokines such as interleukin-2 (IL-2), granulocyte-macrophage colony stimulating factor (GM-CSF), interleukin-12 (IL-12) and interferon-gamma (IFN-gamma), anti-angiogenic molecules and tumor associated antigens (Anderson, et al., 1990, Cancer Res., 50: 1853, Stoklosa, et al., 1998, Ann Oncol., 9:63, Leibson, H. J. et al., 1984, Nature, 309:799, Book, et al., 1998, Semin. Oncol. 1998, 25:381, Salgaller, et al., 1998, J. Surg. Oncol., 68: 122, Griscelli, et al., 1998, Proc. Natl. Acad. Sci. USA, 95: 6367).

### EXAMPLE 6

Treatment of Cancer with Cytokines Anti-angiogenic Molecules or Tumor Associated Antigens Delivered from Implanted Organized Tissue Constructs

Genetically engineered organized tissue constructs are used as platforms for delivering clinically relevant doses of cytokines, anti-angiogenic molecules or tumor associated antigens in cancer patients. Therapeutic doses of anti-tumor molecules can be delivered in a mouse tumor model (Hearing et al., 1986, J. Iminunol., 137: 379). Mouse muscle satellite cells are isolated and plated in T-75 flasks. When the cells are nearly confluent, they are harvested and plated at low density in 35 mm diameter tissue culture plates. The low density cultures are transduced with either a viral or non-viral vector containing the gene encoding human recombinant IL-2 (Gene bank accession #1311005), GM-CSF (Gene bank accession #3169005), IFN-gamma (Gene bank accession #184639), IL-12 (Gene bank accession #2944079), anti-angiogenic molecules or the appropriate tumor associated antigen, as described in Example 2. Transduced cells are engineered into organized tissue constructs as described in Example 1. Following transduction and genetic modification, organoids are implanted under tension in either mice bearing a preestablished tumor or mice which are challenged with a tumor inoculation postimplantation. Mice are anesthetized by methoxyflurane inhalation and the site of incision is shaven and cleaned with 70% ethanol. A 1 cm incision is made, the skin is reflected, and the organoids are inserted (either subcutaneously into a muscle bed or intraperitoneally) and the wound is sutured closed. In vivo tissue or serum levels of recombinant molecules are measured at varying time points following implantation and the effects on tumor development and animal survival are followed over time.

A given cancer treatment according to the invention may be tested in an art accepted animal model of cancer by implanting the organized tissue producing a substance that is bioactive in cancer therapy into the diseased animal and observing clinical parameters over time. Such art-accepted animal models of cancer have been described in Hearing, 1986, J. Immunol., 137:379, Stoklosa et al., 1998, Ann. Oncol., 9:63, Carson et al., 1998, J. Surg. Res., 75:97, Maurer-Gebhard et al., 1998, Cancer Res., 58:2661 and Takaori-Kondo et al., 1998, Blood, 91:4747).

Therapeutic efficacy of treatment of cancer according to the invention by implantation of an organized tissue producing a molecule as described herein, is indicated by changes in clinical parameters such as tumor shrinkage (e.g. at least 5-10% and preferably 25-100%) and/or extended animal survival time. The number of organoids implanted, the number of cells contained within an organoid and the combination of molecules released can be adjusted in order to achieve optimal delivery and beneficial effects. Cancer patients may be treated accordingly by implanting organoids producing cytokines, anti-angiogenic molecules or tumor associated antigens, measuring the level of cytokine, anti-angiogenic molecule or tumor associated antigen, determining tumor size, survival time and the alleviation of symptoms associated with the particular type of cancer being treated over time.

D. Cardiovascular Disorders

The invention also provides methods of treating cardiovascular disorders, including vascular disease, coronary artery disease and congestive heart failure.

Vascular Disease

Vascular disease is a disease related to poor circulation, that is a common complication in patients who have had atherosclerosis or diabetes for a prolonged period of time. Peripheral vascular disease results from hardening, narrowing, or closing off of both the larger and smaller blood vessels in the limbs (commonly the legs), causing foot sores, ulcers, or gangrene. Severe cases of peripheral vascular disease require amputation of the infected limb. Cardiac vascular disease is caused by poor circulation in the heart muscle (often resulting from a heart attack), leading to defective pumping of the heart. If diagnosed early, vascular diseases may be treatable with angiogenic recombinant proteins, such as VEGF (Mack et al., 1998, J. Vasc. Surg., 27:699-709) and/or members of the FGF family (Melillo et al, 1997, Circ Res. 35:80-489). In a rodent (rat) model of peripheral disease, the left common femoral artery is ligated and divided in a hindlimb resulting in ischemia (Mack et al., supra). A similar rodent heart model has been developed wherein myocardial infarction is induced by ligating a coronary artery (Yang et al., 1995, Circulation, 92:262-267). As a result of this procedure vascularity and blood flow are reduced in the affected tissue.

Congestive Heart Failure

Congestive heart failure is a disease related to the inability of the heart to function as an efficient pump. Congestive heart failure is a multiple-etiology disorder, that can result from cardiomyopathy, myocardial infarction, or coronary insufficiency (Yang et al., supra). This disorder is characterized by a decrease in stroke volume and cardiac output. Current treatments for this disease, such as digitalis and angiotensin-converting enzyme inhibitor, can improve the condition of the heart, but do not effectively treat the symptoms of pain and exercise intolerance. A rodent (rat) model of congestive heart failure has been developed wherein myocardial infarction is induced by ligating the left coronary artery (Yang et al., supra). Previous studies have shown that systemic administration of rhGH and/or rhIGF-1 can improve the symptoms of congestive heart failure and improve cardiac performance (Yang et al., supra, Stromer et al., 1996, Circ. Res., 79:227-236).

Coronary Artery Disease

The accumulation of fatty deposits in the cells that line the wall of the coronary artery leading to the obstruction of blood flow, is known as coronary artery disease. As a result of coronary artery obstruction, cardiac ischemia (insufficient blood flow) leading to heart damage can occur. Cardiac ischemia is most commonly caused by coronary artery disease. Angina and heart attack are the major complications of coronary artery disease. Treatment of angina includes administration of beta-blockers, nitrates, calcium antagonists and antiplatelet drugs and, in some cases, angioplasty. Treatment of heart attacks includes reducing the clot in the coronary artery (e.g. by aspirin treatment, thrombolytic therapy, angioplasty or coronary artery bypass surgery) (Andreoli et al., supra and Berkow et al., supra). A method of treatment of coronary artery disease may involve administration of angiogenic proteins such as VEGF (Mack et al., supra) and/or members of the FGF family (Melillo et al., supra).

Cardiomyopathy

The term cardiomyopathy refers to a group of diseases (dilated, hypertrophic and restrictive cardiomyopathy) effecting the heart muscle. Dilated cardiomyopathy is associated with dilation of one or both ventricles of the heart and impaired systolic function. The enlarged ventricles are unable to pump a sufficient amount of blood to the body and as a result, heart failure occurs. The most common cause of dilated cardiomyopathy is coronary artery disease. The symptoms of dilated cardiomyopathy include shortness of breath, increased heart rate, fluid retention in the legs and abdomen, fluid uptake by the lungs, heart murmurs and abnormal heart rhythms. The method of treatment depends on the underlying cause of the dilated cardiomyopathy and may include administration of nitrate, beta-blockers or calcium channel blockers (for individuals with coronary artery disease), administration of anticoagulants to prevent clots, administration of agents that reduce the force of heart contractions or prevent abnormal heart rhythms, treatment with diuretics or administration of digoxin.

Hypertrophic cardiomyopathy is a disease associated with a thickening of the ventricular walls. This condition may be the result of a birth defect, or may occur in individuals with acromegaly or pheochromocytoma. As a result of thickened ventricular walls, there is increased resistance in the heart to blood flowing from the lungs. Consequently, as back pressure develops in the lung veins, fluid accumulates in the lungs causing shortness of breath. The symptoms of hypertrophic cardiomyopathy include faintness, chest pain, palpitations (resulting from irregular heartbeats) and heart failure with shortness of breath. Hypertrophic cardiomyopathy is most commonly treated with beta-blockers or calcium channel blockers.

Restrictive cardiomyopathy refers to disorders wherein the ventricular walls stiffen without thickening, and resist the normal pattern of filling with blood that occurs between heartbeats. When the heart is only partially filled with blood, an inadequate amount of blood can be pumped to an individual engaged in exercise. In one form of restrictive cardiomyopathy a gradual replacement of the heart muscle by scar tissue occurs. The other form of restrictive cardiomyopathy is characterized by infiltration of the heart muscle by material such as white blood cells, not normally found in the heart. The symptoms commonly associated with restrictive cardiomyopathy include heart failure with shortness of breath, tissue swelling (edema), abnormal heart rhythms and palpitations. Restrictive cardiomyopathy can be treated by administering diuretics or by treating the underlying cause of this disorder (Andreoli et al., supra and Berkow et al., supra). A method of treatment of cardiomyopathy may involve administration of GH or inotropic agents (Lombardi et al., 1997, Horm. Res., 48:38 and Cittadini et al., 1997, Endocrin., 138: 5161).

### EXAMPLE 7

Treatment of Vascular Disease with Vascular Endothelial Growth Factor (VEGF) and/or Fibroblast Growth Factor (FGF) Delivered from Implanted Postmitotic Organized Tissue Constructs

Postmitotic organoids are genetically engineered to secrete therapeutic levels of VEGF or FGF to promote angiogenesis in ischemic tissues. Cells (e.g. fibroblasts or myoblasts) are isolated from rats and plated in tissue culture flasks. When the cells are nearly confluent they are harvested and plated at low density in 35 mm diameter tissue culture plates. The low density cells are transduced with the MFG-retroviral vector containing encoding the gene for the human recombinant protein (e.g. VEGF GeneBank Accession No. 117185). Transduction with viral systems is achieved as described in Example 2. Transduced cells are tissue engineered into organoids and implanted in ischemic tissue in mice as described in Example 1. In vivo recombinant protein tissue or serum levels are measured at varying times after implantation by standard radioimmunoassay. Increased blood flow in the affected tissue and increased physiological performance (at least 5-10% and preferably 25-100%) will indicate a positive effect of the treatment. Blood flow to ischemic tissue is evaluated by means of intraarterial administration of 15 μm color microspheres for 20 sec followed by tissue removal, digestion, and sphere per gram tissue counting (Mack et al., supra).

Organoids producing VEGF or EGF may be tested in an animal model of vascular disease (e.g. see Yang et al., supra) by implanting 1 or more organoids. According to this animal model myocardial infarction was produced by left coronary arterial ligation. In brief, under anesthesia (ketamine 80 mg/kg; Aveco Co., Inc.) and xylazine 10 mg/kg IP (Rugby Laboratories, Inc.), rats were intubated via tracheotomy and ventilated by a respirator (Harvard Apparatus model 683). After a left-sided thoracotomy, the left coronary artery was ligated approximately 2 mm from its origin between the pulmonary outflow tract and the left atrium. There was a 40% mortality rate within 48 hours after this procedure. Sham animals underwent the same procedure except that the suture was passed under the coronary artery and then removed (Yang et al., supra).

The therapeutic efficacy of treatment of vascular disease according to the invention by implantation of an organized tissue producing VEGF or EGF as described herein, is indicated by changes in clinical parameters such as an increase in the level of blood flow in ischemic tissues.

Human patients may be treated accordingly by implanting VEGF or EGF-producing organoids, measuring VEGF or EGF levels, blood flow in the ischemic tissue and the alleviation of symptoms of vascular disease at various time points following organoid implantation.

### EXAMPLE 8

Treatment of Congestive Heart Failure with Recombinant Human Growth Hormone (rhGH) and/OR Insulin-like Growth Factors (IGF) Delivered from Implanted Postmitotic Organized Tissue Constructs

Postmitotic organoids genetically engineered to secrete therapeutic levels of rhGH and/or IGF-I are used to improve cardiac output and stroke volume. Cells are isolated from rats and plated separately in tissue culture flasks. When the cells are nearly confluent they are harvested and plated at low density in 35 mm diameter tissue culture plates. The low density cultures are transduced with the MFG-retroviral vector containing the gene for human recombinant GH (e.g. GeneBank Accession No. 134729) or with the MFG-retroviral vector containing the gene for recombinant human IGF-1 (e.g. GeneBank Accession No. 1335140) as described in Example 2. Transduced cells are engineered into organoids as described in Example 1 and rhGH and/or rhlGF-1 secreting organoids are implanted under tension in rats previously undergoing left coronary artery ligation (4 weeks post-operation) as described in Example 3. In vivo rhGH and/or IGF-1 serum levels are measured at varying times after implantation by radioimmunoassay (Perrone et al., 1995, J. Biol. Chem., 270:2099). Improvements in cardiac output and stroke volume (at least 5-10% and preferably 25-100%) will indicate successful treatment with these or other recombinant proteins. Cardiac output is measured by the injection of fluorescent labeled microspheres into the left ventricle. Blood samples from a femoral catheter are collected at varying times and cardiac output calculated as: total # spheres injected x blood flow (0.95 mL/min)/number of spheres in blood sample (Duerr et al., 1996, Circulation, 93:2188). At constant heart rate, the increase in cardiac output is directly proportional to the increase in stroke volume.

Organoids producing rhGH or IGF may be tested in an animal model of congestive heart failure (e.g. see Yang et al., supra) by implanting 1 or more organoids producing rhGH or IGF into the animal and determining the level of rhGH or IGF and the cardiac output and stroke volume of the treated animal over time.

Several animal models have been developed for delivery of sufficient quantities of rhGH or IGF to ameliorate the symptoms of congestive heart failure and increase cardiac performance (Yang et al., supra, described above and Stromer et al., supra).

Human patients with congestive heart failure may be treated accordingly by implanting rhGH or IGF-producing organoids and measuring rhGH or IGF levels, cardiac output and stroke volume and the alleviation of symptoms of congestive heart failure over time.

E. Endocrine Disorders

The invention provides methods of treating endocrine disorders, including diabetes, obesity and growth hormone deficiencies.

Diabetes

Diabetes mellitus is a heterogenous group of four diseases (type I and II diabetes, gestational diabetes and diabetes secondary to other conditions) characterized by high levels of blood glucose resulting from defects in insulin secretion, insulin action, or both. The four different classes of diabetes are thought to have different etiologies but similar pathologic courses following the onset of diabetes.

Insulin dependent or type I diabetes results from an insulin deficiency caused by β-cell destruction. As a result of a decrease in the level of insulin and a concomitant increase in the level of glucagon, there is an increase in glucose production in individuals with type I diabetes. Due to a reduction in the efficiency of peripheral glucose use, plasma glucose levels are increased. As glucose utilization goes down, fat utilization is increased thereby resulting in increased levels of keto acids in the extracellular fluids. The symptoms of type I diabetes include glucose excretion in the urine accompanied by increased excretion of water and salts and frequent urination, increased thirst, changes in catabolism leading to loss of lean body mass, adipose tissue and body fluids, deficits in various intracellular components, and abnormalities of the eye. Treatment of this form of diabetes with insulin results in decreased levels of plasma glucose, free fatty acids, and ketoacids and a reduction in urine nitrogen losses.

Noninsulin-dependent or type 2 diabetes is the most common form of diabetes mellitus and is characterized by impaired insulin-mediated glucose uptake or insulin resistance by the major target tissues. Type II diabetes is frequently associated with obesity. The major symptom of type II diabetes is an elevated fasting level of plasma glucose due to overproduction of hepatic glucose. Treatment of type II diabetes can include caloric regulation, weight reduction if the disease is accompanied by obesity, and the administration of sulfonylurea drugs to improve both tissue responsiveness to endogenous insulin and β-cell responsiveness to glucose. Insulin injections are required for treating the late stages of the disease (Beme and Levy et al., supra). Leptin may also be useful for the treatment of diabetes via regulation of the levels of blood glucose and fat (Murphy et al., 1997, Proc. Natl. Acad. Sci. USA, 94:13921)

Obesity

Obesity is defined as an accumulation of excessive body fat. Individuals are considered obese if their weight is 20% or more over the midpoint of their weight range according to a standard height-weight table. Obesity occurs when the consumption of calories exceeds calorie usage by the body. Mechanistically, obesity is caused either by a failure of adipose cells to send signals to the brain (thereby regulating food seeking and consumption behavior) or failure of the brain to respond to signals from adipose tissue in an appropriate manner. To a large degree obesity is genetically predetermined.

Obese individuals may experience poorly regulated glucose in the blood, breathing difficulties, shortness of breath and severe respiratory problems resulting from pressure being exerted on the lungs from excess fat accumulated below the diaphragm and in the wall of the chest. Kidney problems, orthopedic problems, skin disorders and edema may also be associated with obesity. Methods of treatment of obesity include severely decreased caloric intake and surgery to reduce stomach size (Andreoli et al., supra and Berkow et al., supra). Obesity may also be successfully treated by regulating the levels of blood glucose and fat with leptin and/or insulin. The genetically obese mouse represents an animal model for diabetes and obesity (Murphy et al., 1997, Proc. Natl. Acad. Sci USA, 94: 13921-13926).

Growth Hormone Insufficiency

Growth hormone is a single-chain protein with a molecular weight of 22,000 that is normally produced by a pituitary gene. The synthesis of growth hormone is regulated by growth hormone releasing hormone, thyroid hormone and cortisol. Growth hormone secretion can be stimulated by a variety of factors (e.g. a decrease in the levels of glucose or fatty acids, fasting, exercise or estrogens), and inhibited by various factors (e.g. somatostatin, an increase in the level of glucose or fatty acids, or growth hormone).

A number of mechanisms including hypothalamic dysfunction, pituitary tumors, an inactive growth hormone protein, decreased production of peptide hormone mediators of growth hormone action (e.g. somatomedins) or receptor abnormalities, can result in a growth hormone deficiency in children. The physiological manifestations of a growth hormone deficit in children include short stature (for example Turner's Syndrome), delayed bone maturation, mild obesity, and delayed puberty. Turner's Syndrome is a gonadal disorder affecting females in which their is partial or total loss of one of the X-chromosomes. This disease is characterized by short stature, and various somatic anomalies including epicanthal folds, low-set ears, webbed neck, multiple pigmented nevi, lymphedema of the hands and feet, renal malformations and coarctation of the aorta (Andreoli et al., supra and Berkow et al., supra). Treatment with growth hormone can result in increased nitrogen retention, increased lean body mass, decreased adipose mass, increased growth speed (in children), the initiation of puberty and the establishment of fertility (Berne and Levy, supra).

Dwarfism can be caused by a decrease in growth hormone secretion that is most commonly due to a hereditary defect. Another less common form of dwarfism is caused by a failure of the anterior pituitary gland to secrete growth hormone. The physical characteristics of a pituitary dwarf include a failure to demonstrate normal organ and bone growth, repressed sexual development, and short stature (Guyton, supra). Dwarfism in humans results in many instances from reduced growth hormone (GH) secretion from the brain's pituitary gland (Daughaday et al., 1995, In Growth Hormone, Harvey et al., eds., CRC Press Inc., Boca Raton, 475-504). In an animal model of this disease, growth deficient rats (dwarf DW4 rats) are approximately 40% smaller than age-matched normal rats due to expression of pituitary GH at levels that are 5-10% of normal (Charlton et al., 1988, J. Endocrinol., 119: 51-58).

### EXAMPLE 9

Treatment of Diabetes and Obesity with Recombinant Protein Delivered from Implanted Postmitotic Organized Tissue Constructs

Postmitotic, organized connective tissue fibroblast-like organs (organoids) genetically engineered to secrete therapeutic levels of recombinant proteins such as insulin or leptin are used to treat diabetes and/or obesity by controlling blood glucose and/or fat.

Fibroblasts are isolated from the connective tissue of individual rats and plated separately in T-75 flasks. When the cells are nearly confluent they are harvested and plated at low density in 35 mm diameter tissue culture plates. Analysis of cell shape and immunocytochemical methods are used to determine the percentage of the cells that are fibroblasts. Fibroblasts are stellate in shape and should not stain positively for desmin, an intermediate filament protein found only in myoblasts. The low density cultures are transduced with the MFG-insulin or MFG-leptin retroviral vector (containing the gene encoding for either insulin (GeneBank Accession #2098404) or leptin (GeneBank Accession #1469860) as described in Example 2. Transduced fibroblasts are engineered into fibroblast organoids for each individual animal (i.e autologous implants) as described in Example 1. The amount of insulin or leptin secreted from the organoids in vitro is quantitated by standard radioimmunoassays (Murphy et al., supra).

A given method of treatment for obesity and diabetes according to the invention may be tested in an art accepted animal model of obesity and diabetes by implanting the organized tissue producing a substance that is bioactive in obesity and diabetes therapy into the diseased animal and observing clinical parameters over time. Such art-accepted animal models of obesity and diabetes include the genetically obese mouse (Murphy et al., supra).

The ob/ob mouse is genetically deficient in leptin and exhibits a phenotype that includes obesity and non-insulin-dependent diabetes mellitus. This phenotype closely resembles the morbid obesity seen in humans. In ob/ob mice, mutation in the ob gene leads to a marked increase in food consumption that results in an increase in adipose tissue mass and a syndrome that resembles morbid obesity in humans. Abnormalities include hypothermia, lethargy, hyperglycemia, glucose intolerance, and hyperinsulinemia resembling non-insulin-dependent diabetes melitus in humans (Murphy et al., supra).

Therapeutic efficacy of treatment of obesity and diabetes according to the invention by implantation of an organized tissue producing a molecule as described herein, is indicated by changes in clinical parameters such as an increase in glucose tolerance and a decrease in food intake and body weight. Glucose tolerance is determined by injecting glucose I.P. into fasted individuals and monitoring circulating glucose in blood samples collected every 30 minutes for 4 to 6 hours. Glucose levels are measured using a Lifespan One Touch Monitor (Mountainview, Calif.). Significant decreases of at least 10%, and preferably 15-50%, in blood glucose levels, food intake, and/or body weight compared to controls will be considered acceptable to show activity of the recombinant protein.

Diabetes patients and obese patients may be treated accordingly by implanting organoids producing insulin and/or leptin, measuring the level of insulin and/or leptin, determining blood glucose levels and weight loss and the alleviation of symptoms associated with obesity and diabetes over time.

### EXAMPLE 10

Treatment of Dwarfism with Insulin-like Growth Factor-1 Delivered from Implanted Fibroblast Organized Tissue Constructs

Organized connective tissue fibroblast-like organs (organoids) genetically engineered to secrete therapeutic levels of IGF-1 were used to stimulate animal growth in a dwarf animal, and bypass the need for increasing GH levels.

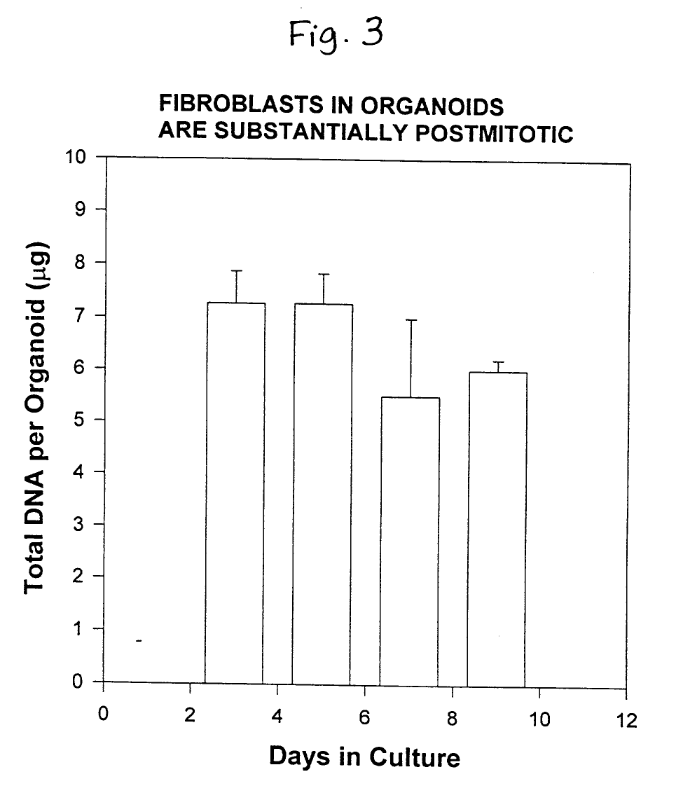

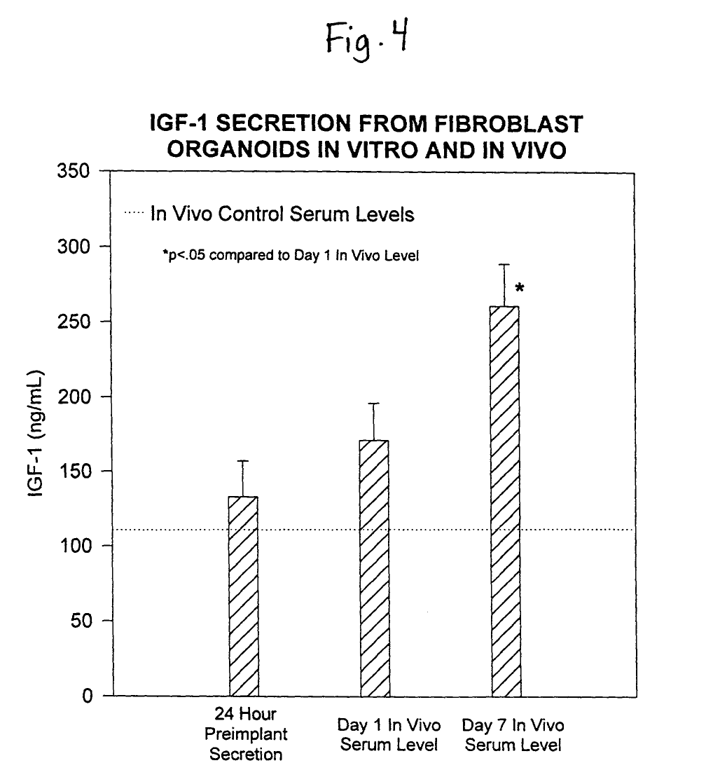

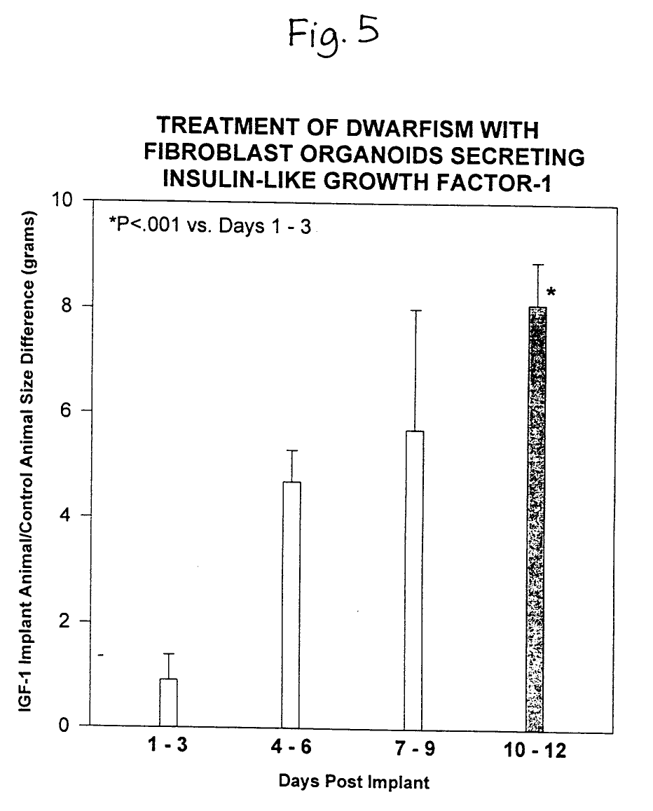

Fibroblasts were isolated from the connective tissue of individual female dwarf rats and plated separately in T-75 flasks. When the cells were nearly confluent they were harvested and plated at low density in 35 mm diameter tissue culture plates. Nearly 100% of the cells were fibroblasts, based on cell shape (stellate), and were non-myoblasts as indicated by lack of positive immunocytochemical staining for desmin, an intermediate filament protein found only in myoblasts. The low density cultures were transduced with the MFG-IGF-1 retroviral vector (containing the gene encoding for human recombinant IGF-1) as described in Example 2. Transduced fibroblasts were engineered into fibroblast organoids for each individual animal (i.e autologous implants) as described in Example 1. Cells in the fibroblast organoids aligned parallel to the axis of the tubing, were postmitotic and contained constant levels of DNA after the first day in culture (FIG. 3). In vitro, transduced fibroblast organoids secreted approximately 20 ng/mL IGF-1/day/organoid compared to less than 5 ng/mL IGF-1/day/organoid secreted by control, nontransduced organoids (data not shown). Up to ten IGF-1 secreting autologous fibroblast organoids were implanted under tension in dwarf rats, (as described in Example 3). In vivo IGF-1 serum levels were measured on Days 1 and 7 after implantation and showed a significant 53% increase from 171±25 to 261±28 ng/mL by Day 7 (FIG. 4). The increase in circulating IGF-1 serum levels in the dwarf rats was adequate to produce a significant increase in animal size over the ten to twelve day period following fibroblast organoid implantation (FIG. 5).

A given treatment for dwarfism according to the invention may be tested in an art accepted animal model of dwarfism by implanting the organized tissue producing insulin-like growth factor 1 into the diseased animal and observing clinical parameters over time. An art-accepted animal model of dwarfism includes but is not limited to growth deficient, dwarf DW4 rats (Charlton et al., supra).

A mutant dwarf rat bearing a mutation, inherited as an autosomal recessive, arose spontaneously in a breeding colony of Lewis rats. This dwarf rat has been characterized. Body growth in the mutant is retarded such that at 3 months of age both males and females weigh approximately 40% less than their normal litter-mates, and continue to grow at a slower rate. The mutants show a selective reduction in pituitary GH synthesis and storage (pituitary GH concentrations were approximately 10% of normal in males and 6% in females). The concentration of their anterior pituitary trophic hormones (LH, TSH, prolactin and ACTH) were within the normal range in dwarf animals. This model has been used to demonstrate the therapeutic efficacy of growth hormone (administered by injection) in the treatment of dwarfism. Exogenous GH treatment for 5 days resulted in an increase in growth rate from 1.5±0.3 to 3.1±0-4 g/day in male mutants, and 0.8±0.2 to 3.1±0.1 g/day in females. Longitudinal bone growth rates were more than doubled by this treatment from 49±5 to 100±10 μm/day in females and from 52±11 to 131±16 μm/day in males (Charlton et al., 1998).

Therapeutic efficacy of treatment of dwarfism according to the invention by implantation of an organized tissue producing a molecule as described herein, is indicated by changes in clinical parameters such as animal size (e.g. at least 1-5% and preferably 10-60%).

Human dwarfism patients may be treated accordingly by implanting organoids producing IGF-1, measuring the level of IGF-1, determining changes in the patient size, and the alleviation of symptoms associated with dwarfism over time.

F. Immune Disorders

The invention provides a method of treating immune disorders including Chronic granulomatous disease (CGD), acute/chronic renal failure, severe combined immunodeficiency and autoimmune disorders. The invention also provides a method of delivering a composition useful for vaccination (e.g. against whooping cough).

Chronic Granulomatous Disease

CGD is a recessive disorder characterized by a defective phagocyte respiratory burst oxidase, life-threatening pyogenic infections and inflammatory granulomas (Pollock et al., 1995, National Genetics, 9:202-209). Methods of treating CGD with recombinant proteins such as gamma interferon are designed to maintain a constant level of recombinant protein in the bloodstream. In one animal model of this disease, Mycobacterium marinum caused CGD in immunocompetent leopard frogs (Rana pipiens) (Ramakrishnan et al., 1997, Infectious Immunology, 65:767-773). Another animal model for CGD is a knock out mouse wherein a mouse contains a null allele of a gene involved in X-linked CG (the 91 kD subunit of oxidase cytochrome b) (Pollock et al., supra).

Acute or Chronic Renal Failure

Kidney failure is defined as an inability of the kidney to filter blood and excrete toxic substances from the body. Acute kidney failure refers to a rapid loss of kidney function and is often associated with multiple organ failure and sudden death. Chronic kidney failure is defined as a gradual and progressive deterioration of kidney function often associated with diabetes and high blood pressure.

The rapid decline in the ability of the kidney to remove toxic substances from the blood that occurs during acute kidney failure, results in an increase in the level of nitrogenous waste products (e.g. urea) in the blood. Acute kidney failure can be caused by any condition that i. results in a reduction in the flow of blood to the kidney, ii. interferes with the flow of urine after it has left the kidneys, or iii. produces an injury to the kidney. The symptoms associated with acute kidney failure are variable and depend on the initial cause of kidney damage. Often, a condition that results in acute renal failure may produce symptoms unrelated to the kidneys, including high fever, shock and heart failure. Symptoms of acute renal failure resulting from an obstruction of urine flow may include cramping, resulting from stretching of the urine collecting area, and blood in the urine. Decreased urine output, as well as increased levels of creatinine, urea, acid, potassium and decreased sodium in the blood, can be indicative of acute kidney failure. Acute kidney failure can be successfully treated by restricting water intake, administration of particular amino acids to maintain a sufficient protein level, restricting the uptake of substances that are eliminated through the kidney, administration of antacids to prevent increases in the blood phosphorous levels, administration of polystyrene suflonate to treat high potassium levels, or dialysis. Acute renal failure may also be successfully treated with recombinant proteins such as human hepatocyte growth factor (HGF) (Goto et al., 1997, Nephron, 77:440). Human alpha-galactosidase A will prevent the progressive deposition of neutral glycosphingolipids in vascular endothelial cells that causes renal failure (Ohshima et al., 1997, Proc. Natl. Acad. Sci. USA,94:2540-2544) and may be useful for the treatment of acute renal failure.

Another recombinant protein called OP-1 (U.S. Pat. No. 5,650,276 and U.S. Pat. No. 5,707,810) is found to protect against kidney damage in animal models of acute and chronic renal failure and may be useful for the treatment of these disorders. OP-1 has been shown to improve the blood flow and filtration in kidneys, thereby reducing toxin accumulation in the bloodstream. OP-1 also reduces the level of expression of certain markers of inflammation. In an animal model of renal failure, a portion of the kidney is removed from nude mice in a two-step nephrectomy procedure in order to simulate a renal failure scenario (Hamamori et al., 1995, J. Clinical Investigation, 95:1808-1813)

The slow, progressive, and irreversible loss of kidney function that is associated with chronic kidney failure, causes an increase in the level of nitrogenous waste products in the blood. Symptoms are slow to develop in an individual suffering from chronic renal failure and can include increased urination, high blood pressure, possibly leading to stroke or heart failure. During the later stages of kidney failure, an increase in the level of toxic substances in the blood can cause fatigue, nerve and muscle symptoms (e.g. twitching and muscle weakness), seizures, digestive tract abnormalities, ulcers and skin disorders. Blood tests that detect increased levels of urea and creatinine or a state of acidosis can be used to diagnose chronic renal failure. Most methods of treating chronic renal failure cannot prevent the progression of this disease. In an individual with chronic renal failure, sodium, water and acid imbalances should be corrected, substances that are toxic to the kidney should be removed, and heart failure, high blood pressure, infections, increased levels of blood potassium or calcium and obstructed urine flow should be treated. If these modes of treatment are ineffective, long-term dialysis or kidney transplantation may be considered as appropriate methods of treatment (Andreoli et al., supra and Berkow et al., supra).

Severe Combined Immunodeficiency Disease (SCID)

SCID results from a deficiency in immunocompetent T and B cells, resulting in severe and persistent infections beginning in the early stages of life. About half of all SCID patients harbor a deficiency in the purine salvage enzyme, adenosine deaminase (ADA). These patients have single base pair mutations in the ADA gene that result in amino acid substitutions, and, in some cases, either a splicing mutation or a deletion (Hirschorn, 1990, Immunodeficiency Review, 2:175-198). Treatment of this form of recessive SCID with adenosine deaminase (ADA) injections is possible. Some SCID patients have an X-linked mutation in the IL-R gamma chain, and treatment of this disease with IL-2 and IL-2R gamma chain may prove to be successful (Leonard et al., 1994, Immunology Review, 138:61-86). Animal models of SCID include a canine model of XSCID, the most common form of human SCID in the United States, and an equine model of an autosomal recessive form of SCID, (Felsburg et al., Immunodeficiency Review, 3:277-303). Other animal models for SCID include SCID mice and nude mice (Ye and Chiang et al., 1998, Clin. Exp. Rheum., 16:33 and Sandhu et al., 1996, Crit. Rev. Biotechnol., 16:95).

Vaccination

Vaccination is a commonly used method for creating a state of immunity against a specific disease in an individual. Vaccinations can comprise i. dead organisms that retain antigenicity but are no longer capable of inducing disease (useful for treating typhoid fever, whooping cough, diphtheria and other bacterial diseases), ii. toxins that have been chemically treated such that they are antigenic but non-toxic (useful for treating tetanus, botulism, and other toxic diseases), or iii. live organisms that have been mutated such that they do not cause disease but remain antigenic (useful for protection against poliomyelitis, yellow fever, measles, smallpox, and other viral diseases (Guyton, supra).

Whooping cough is a respiratory infection caused by Bordetella pertussis, an organism which produces a wide array of factors that contribute to the development of the disease. The expression and regulation of these virulence factors is dependent upon the bvg locus (originally designated the vir locus), which encodes two proteins: BvgA, a 23-kDa cytoplasmic protein, and BvgS, a 135-kDa transmembrane protein (Merkel et al., 1998, Journal of Bacteriology, 180: 1682-90). Immunization against whooping cough with acellular Bordetella pertussis fragments can confer future protection against whooping cough Ryan et al., 1998, Immunology, 93: 1). Mice with specific disruptions in their B-cell genes (gamma interferon receptor, interleukin 4, or immunoglobulin heavy-chain genes) are shown to be a reliable animal model for studying whooping cough vaccination (Mills et al., 1998, Infectious Immunology, 66:594-602). The murine respiratory challenge model is also a useful model for studying whooping cough vaccination. This model has been used to examine the local T cell responses in the lung during infection with Bordetella pertussis (McGuirk et al., 1998, Eur-J-Immunol., 28: 153-63).

Multiple Sclerosis

Multiple sclerosis (MS) is a central nervous system disease characterized by plaques of demyelination in nerve fibers of the brain and spinal cord. Demyelination causes multiple and varied neurologic symptoms and signs such as neurologic dysfunction including abnormal movement, abnormal sensations, tingling and numbness, loss of strength or dexterity, and visual abnormalities. The physical manifestations of multiple sclerosis result from the demyelination process slowing or blocking the conduction of nerve impulses. MS is typically characterized by periods of relapses and remissions, and eventually becomes progressive in most patients. Although the etiology of multiple sclerosis is not known, it is thought that this disease is caused by both immunologic and genetic factors. The most sensitive method for diagnosing multiple sclerosis is magnetic resonance imaging to detect a loss of myelin as white matter lesions located in the brain and/or spinal cord (Berkow et al., supra).

Currently methods exist for treating the symptoms of multiple sclerosis rather than the disease. The frequency of relapses associated with multiple sclerosis can be decreased with beta-interferon treatment. Beta-interferon also reduces the rate of appearance of cerebral demyelinating lesions. Corticosteroids have also been used to treat multiple sclerosis (Berkow et al., supra). Another protein that may be useful for the treatment of multiple sclerosis is the neuroprotectant molecule annexin-1, a calcium-dependent phospholipid binding protein. A useful animal model for MS is provided by female SJL/J mice with experimental autoimmune encephalomyelitis (EAE), a disease that exhibits symptoms that mimic MS (Ding et al., 1998, J. Immunol., 160: 2560-2564).

Autoimmune Disorders

In some instances, individuals can suffer a loss of immune tolerance to some of their own tissues. Often this results from destruction of some of the body's tissues leading to release of antigens, their circulation in significant quantities in the body fluids, and the production of antibodies directed against these antigens. Autoimmune diseases are characterized by the abnormal production of antibodies reactive against self components.

Diseases that result from autoimmunity include autoimmune hemolytic anemia caused by the production of antibodies against the bodies own erythrocytes, rheumatic fever wherein exposure to a specific type of streptococcal toxin causes the body to become immunized against tissues in the heart and joints, acute glomerulonephritis wherein exposure to a streptococcal toxin causes an individual to become immunized against the glomeruli, myasthenia gravis wherein the body develops an immunity to muscles that subsequently results in paralysis, and lupus erythematosus wherein an individual becomes immunized against multiple tissues simultaneously and suffers extensive damage, often resulting in rapid death (Guyton, supra).

### EXAMPLE 11

Treatment of Chronic Granulomatous Disease with Gamma Interferon Delivered from Implanted Organized Tissue Constructs

Organized nonproliferative tissue constructs genetically engineered to secrete therapeutic levels of gamma interferon are used to stimulate the antimicrobial mechanisms of blood monocytes, circulating neutrophils and tissue macrophages (Murray, 1996, Intensive Care Medicine, 22 Suppl.4 S456-461).

Cells (e.g. myoblasts or fibroblasts) are isolated from animals and plated separately in tissue culture flasks. When the cells are nearly confluent they are harvested and plated at low density in 35 mm diameter tissue culture plates. The low-density cultures are transduced with the MFG-retroviral vector containing the recombinant protein gene (e.g. gamma interferon, GENBANK Accession #1568222) as described in Example 2. Transduced cells are engineered into organized tissue constructs as described in Example 1. It is expected that transduced cells in organoids will secrete significantly greater amounts of gamma interferon in vitro than nontransduced control constructs. Up to ten gamma interferon secreting constructs are implanted under tension in animals as described in Example 1 (mice) or Example 3 (rats). The in vivo serum levels of gamma interferon serum are measured at varying times after implantation and should be significantly increased as compared to the levels in animals transplanted with non-gamma interferon secreting tissue constructs.

A given treatment for CGD according to the invention may be tested in an art accepted animal model of CGD by implanting the organized tissue producing gamma interferon into the diseased animal and observing clinical parameters over time. Art-accepted animal models of CGD include but are not limited to CGD induced by Mycobacterium marinum in immunocompetent leopard frogs (Ramakrishnan et al., supra) and a CGD knock out mouse expressing a null allele for a subunit of oxidase cytochrome b (Pollock et al., supra).

As described in Ramakrishnan et al. frogs of the species Rana pipiens infected with three different strains of M. Marinum developed a chronic granulomatous disease. A chronic nonlethal granulomatous infection was produced unless the frogs were immunosuppressed by the administration of hydrocortisone, in which case acute fulminant disease developed (Ramakrishnan et al., supra).

According to the method of Pollack et al. mice with a non-functional allele for the gp91^{phox} subunit of the phagocyte oxidase cytochrome b were generated by using targeting homologous recombination in murine embryonic stem (ES) cells. Respiratory burst oxidase activity was absent in neutrophils and macrophages obtained from affected hemizygous male mice. These mice also exhibit an increased susceptibility to infection with S. Aureus and A. Fumigatus and had increased numbers of peritoneal exudate neutrophils during the chemical peritonitis induced by thioglycollate. The murine gp91^{phox} gene, as is its human counterpart, is located on the X chromosome at a locus designated as Cybb. A 4.8 kilobase (kb) NcoI genomic fragment containing the second and third exons of gp91^{phox} gene was used to construct a gene targeting vector by placing an expression cassette for neomycin-resistance into the third exon and attaching a flanking herpes thymidine kinase gene. One of 380 G418-and gancyclovir-resistant ES cell clones isolated after electroporation of the targeting vector displayed correct targeting of the gp₉₁^{phox} gene. This clone gave germline transmission in three different chimeric males generated by blastocyst injection. These chimeric males all had a subpopulation of circulating neutrophils devoid of respiratory burst oxidase activity as measured by a histochemical assay of respiratory burst activity, the nitroblue tetrazolium (NBT) test, suggesting that the targeting gp91^{phox} gene was non-functional. Carrier females generated from breeding blastocyst injection chimaeric males to C57BI/6J females had a mixture of both NBT-positive and NBT-negative peripheral blood neutrophils, consistent with X inactivation of the Cybb locus. Male mice hemizygous for the targeting gp91^{phox} gene, (X-CGD mice), were generated by breeding female carriers to wild-type C57BI/6J males. Cells obtained from X-CGD mice has no detectable gp91^{phox} protein. The p22^{phox} subunit of the phagocyte cytochrome b was also not detected in X-CGD cells (Pollack et al., supra).

Therapeutic efficacy of treatment of CGD according to the invention by implantation of an organized tissue producing gamma interferon as described herein, is indicated by changes in clinical parameters such as a change in superoxide production (at least 5-10% and preferably 25-100%). Flow cytometric procedures for semi-quantitating superoxide production in neutrophils have been developed to evaluate neutrophil function. This procedure, which requires only a small amount of blood, can easily and rapidly yield reproducible and reliable data and is expected to be clinically useful for diagnosis of patients with impaired neutrophil function (Ishikawa et al., 1997, 45:1057).

Human CGD patients may be treated accordingly by implanting organoids producing gamma interferon, measuring the level of gamma interferon, measuring superoxide production, and the alleviation of symptoms associated with CGD over time.

### EXAMPLE 12

Treatment of Acute Renal Failure with Recombinant Proteins Delivered from Implanted Organized Tissue Constructs.

Organized tissue constructs genetically engineered to secrete therapeutic levels of a recombinant protein which has mitogenic activity for various epithelial cells including renal epithelial cells, and accelerates tissue regeneration (Karger et al., supra) are used to treat acute renal failure.

Cells (e.g. myoblasts and fibroblasts) are isolated from animals and plated separately in tissue culture flasks. When the cells are nearly confluent they are harvested and plated at low density in 35 mm diameter tissue culture plates. The low-density cultures are transduced with the MFG-retroviral vector containing the gene for a recombinant protein (e.g. human recombinant hepatocyte growth factor, GENBANK Accession #219700) as described in Example 2. Transduced cells are engineered into organized tissue constructs as described in Example 1. The amount of in vitro secretion of hepatocyte growth factor by transduced cells in constructs should be significantly greater than the amount secreted by nontransduced control constructs. One or more HGF secreting constructs are implanted under tension in animals as described in Example 1 (mice) or Example 3 (rats). The in vivo serum level of hepatocyte growth factor is measured at varying times after implantation by standard radioimmunoassay.

A given treatment for acute renal failure according to the invention may be tested in an art accepted animal model of acute renal failure by implanting the organized tissue producing recombinant proteins (e.g. hepatocyte growth factor) into the diseased animal and observing clinical parameters over time. An art-accepted animal model of acute renal failure includes but is not limited to nude mice subjected to two-step nephrectomy (Hamamori et al., supra, described above).

Therapeutic efficacy of treatment of acute renal failure according to the invention by implantation of an organized tissue producing hepatocyte growth factor as described herein, is indicated by changes in clinical parameters such as increased filtration capacity of the kidney (e.g. at least 5-10% and preferably 25-100%). Insulin clearance is used to quantify the kidney's ability to excrete various substances. Renal clearance of a substance equals urinary excretion rate divided by its plasma concentrations as given by the formula C=U×V/P (Guyton and Hall, 1996, Textbook of Medical Plysiologygy, 9th edition, W.B. Saunders Company).

Human patients with renal failure may be treated accordingly by implanting organoids producing hepatocyte growth factor, measuring the level of hepatocyte growth factor, determining changes in kidney filtration capacity, and the alleviation of symptoms associated with acute renal failure over time.

### EXAMPLE 13

Treatment of SCID with ADA Delivered from Implanted Organized Tissue Constructs.

Organized tissue constructs genetically engineered to secrete therapeutic levels of ADA are used to treat SCID.

Cells (e.g. myoblasts or fibroblasts) are isolated from animals and plated separately in tissue culture flasks. When the cells are nearly confluent they are harvested and plated at low density in 35 mm diameter tissue culture plates. The low-density cultures are transduced with the MFG-retroviral vector containing the gene for a recombinant protein (e.g. human recombinant ADA, GENBANK Accession #'s 178075, 178077, 178079 or IL-2R gamma chain GENBANK Accession #33813, as described in Example 2. Transduced cells are engineered into organized tissue constructs as described in Example 1.

Organized constructs comprising transduced cells should secrete significantly greater amounts of ADA (or IL-2R gamma chain) than nontransduced control constructs. Up to ten ADA (or IL-2R gamma chain) secreting constructs are implanted under tension in animals as described in Example 1 (mice) or Example 3 (rats). The serum level of adenosine deaminase (or IL-2R gamma chain) in vivo is measured at varying times after implantation and should be significantly increased as compared to animals implanted with non-ADA (or non-IL-2R gamma chain) secreting tissue constructs. The level of specific activity of ADA can be assayed as described in Katsir et al., 1998, Bioelectromagnetics, 19:46. The level of IL-2R gamma chain can be measured by sandwich ELISA techniques (Nielson et al., 1998, Am. J Gastroenterol., 93:295).

A given treatment for SCID according to the invention may be tested in an art accepted animal model of SCID by implanting the organized tissue producing ADA (or IL-2R gamma chain) into the diseased animal and observing clinical parameters over time. Art-accepted animal models of SCID include but are not limited to canine models of XSCID and equine models of SCID (Felsburg et al., supra) and SCID and nude mice (Ye and Chiang, supra, Sandhu et al., supra).

Canine X-linked SCID (XSCID) has an X-linked recessive mode of inheritance and, as such, represents a model for the most common form of human SCID in the United States. The canine model of an X-linked form of SCID (XSCID) in the dog that has very similar clinical, immunologic and pathologic features as XSCID in children. Affected dogs have normal or elevated percentages of circulating B cells and an absence of mature, and low to normal percentages of phenotypically mature, but nonfunctional T cells as observed in XSCID boys. Severe combined immunodeficiency in the horse is an autosomal recessive form of SCID that is characterized by a profound lymphopenia causing a marked deficiency in both function and number of B and T cells, most likely due to a lymphoid stem cell defect. Since these diseases are naturally-occurring in an outbred species, like man, they represent unique animal models of their respective human counterparts in which to determine the underlying immunologic defects(s), to evaluate novel approaches to immunotherapy or gene therapy, and to evaluate therapeutic regimens for opportunistic infections associated with SCID (Felsburg et al., supra).

Therapeutic efficacy of treatment of SCID according to the invention by implantation of an organized tissue producing ADA (or IL-2R gamma chain) as described herein, is indicated by changes in clinical parameters such as disease fighting capability as described in van-Tol et al., 1998, Bone-Marrow Transplant, 21:497.

Human SCID patients may be treated accordingly by implanting organoids producing ADA, measuring the level of ADA (or IL-2R gamma chain), determining changes in disease fighting capability, and the alleviation of symptoms associated with SCID over time.

### EXAMPLE 14

Treatment of Whooping Cough with Acellular Pertussis Antigenic Fragments Delivered from Implanted Organized Tissue Constructs

Prevention of whooping cough with acellular pertussis antigenic fragments delivered from implanted nonproliferative organized tissue constructs results in increased levels of antibodies directed against pertussis. Organized tissue constructs genetically engineered to secrete constant level of pertussis antigens into the animal blood stream are used to treat whooping cough.

Cells (e.g. myoblasts or fibroblasts) are isolated from animals and plated separately in tissue culture flasks. When the cells are nearly confluent they are harvested and plated at low density in 35 mm diameter tissue culture plates. The low-density cultures are transduced with the MFG-retroviral vector containing the genes for Bordetella pertussis surface antigens (e.g. GENBANK Accession #s 2120994, 2120995) as described in Example 2. Transduced cells are engineered into organized tissue constructs as described in Example 1. In vitro, transduced cells in constructs should secrete significantly greater amounts of Bordetella pertussis surface antigens than nontransduced control constructs. Up to ten pertussis antigen secreting constructs are implanted under tension in animals as described in Example 1 (mice) or Example 3 (rats). The in vivo levels of antibody directed against pertussis antigen are measured at varying times after implantation by ELISA (Rennels et al., 1998, Pediatrics, 101:604 and Simondon et al., 1998, Clin. Diagn. Lab. Immunol., 5:130) and should demonstrate a significant increase as compared to animals implanted with non-pertussis antigen secreting tissue constructs.

A given treatment for whooping cough according to the invention may be tested in an art accepted animal model of whooping cough by implanting the organized tissue producing acellular pertussis antigenic fragments into the diseased animal and observing clinical parameters over time. Art-accepted animal models of whooping cough include but are not limited to mice with B-cell genes (e.g. gamma interferon receptor, interleukin 4 or immunoglobulin heavy-chain genes) containing specific disruptions (Mills et al., supra) or the murine respiratory challenge model (Mills et al., supra and McGuirck et al., supra).

According to the respiratory challenge model, respiratory infection of mice was initiated by the following method. B. Pertussis W28 phase I was grown under agitation conditions at 37° C. in Stainer-Scholte liquid medium. Bacteria from a 48-h culture were resuspended at a concentration of approximately 2×10¹⁰ CFU/ml in physiological saline containing 1% casein. The challenge inoculum was administered to mice as an aerosol over a period of 15 min by means of a nebulizer (Mills et al., supra).

Therapeutic efficacy of treatment of whooping cough according to the invention by implantation of an organized tissue producing acellular pertussis antigenic fragments as described herein, is indicated by changes in clinical parameters (a descrease of at least 5-10% and preferably 25-100%) such as the level of specific immunoglobulin G or A against the pertussis toxin or against filamentous hemmagglutinin (Simondon et al., supra).

Human whooping cough patients may be treated accordingly by implanting organoids producing acellular pertussis antigenic fragments, measuring the level of acellular pertussis antigenic fragments, determining changes in the level of specific immunoglobulin G or A against the pertussis toxin, and the alleviation of symptoms associated with whooping cough over time.

### EXAMPLE 15

Treatment of Multiple Sclerosis with Interferon Beta 1-A Delivered from Implanted Organized Tissue Constructs

Organized tissue constructs genetically engineered to secrete human interferon beta 1-A are used to treat multiple sclerosis.

Cells (e.g. myoblasts or fibroblasts) are isolated from animals and plated separately in tissue culture flasks. When the cells are nearly confluent they are harvested and plated at low density in 35 mm diameter tissue culture plates. The low-density cultures are transduced with the MFG-retroviral vector containing the gene for a recombinant protein (e.g. human interferon beta 1-A, GENBANK Accession# 386802) as described in Example 2. Transduced cells are engineered into organized tissue constructs as described in Example 1. It is expected that in vitro, transduced cells in organoids will secrete significantly greater amounts of human interferon beta 1-A than nontransduced control constructs. Up to ten human interferon beta 1-A constructs are implanted under tension in animals as described in Example 1(mice) or Example 2 (rats). The in vivo serum levels of human interferon beta 1-A are measured at varying times after implantation by ELISA (Mazzoran et al., Ital. J. Gastroenterol. Hepatol., 29:338) and should be significantly increased as compared to the serum levels of animals implanted with non-human interferon beta 1-A secreting, control tissue constructs.

A given treatment for MS according to the invention may be tested in an art accepted animal model of MS by implanting the organized tissue producing interferon beta 1-A into the diseased animal and observing clinical parameters over time. An art-accepted animal models of MS is provided by female SJL/J mice with experimental autoimmune encephalomyelitis disease (Ding et al., 1997, J. Neuroimmunol., 77: 99). According to this model, a hemiparkinsonian model -15 was created by unilateral intracarotid injection of 0.3 to 0.6 mg/kg of 1-methyl-4-phenyl-1,2,3,6-tetrahydropyridine (MPTP) in approximately 15 cc of 0.9% normal saline at a rate of 1.0 ml/min. Sterile, open microsurgical procedures were performed to allow retrograde injection of the MPTP solution through 26-gauge needles placed in the right common carotid artery after permanent ligation of the external carotid artery and its proximal branches. (Aebischer et al., 1994, supra).

Therapeutic efficacy of treatment of MS according to the invention by implantation of an organized tissue producing interferon beta 1-A as described herein, is indicated by changes in clinical parameters such as the following. Serial magnetic resonance has become an important tool in monitoring treatment efficacy for multiple sclerosis. It provides data which can be readily analyzed in a blinded fashion and which directly inspects the pathological evolution; it also enables a rapid and sensitive measure of treatment outcome in early relapsing-remitting and secondary progressive disease (Miller et al., 1998, Brain, 121 (Pt 1):3). Determination of changes in the number of active magnetic resonance imaging lesions and in the volume of lesions (at least 5-10% and preferably 25-100%) (by monthly gadolinium-enhanced MRI wherein the number of active lesions serves as the outcome measure) can also be used as a measure of the therapeutic efficacy of a method of treatment of MS.

Human MS patients may be treated accordingly by implanting organoids producing interferon beta 1-A, measuring the level of interferon beta 1-A, determining changes the number and volume of magnetic resonance lesions, and the alleviation of symptoms associated with MS over time.

G. Infectious Disease

The invention provides methods of treating infectious diseases including but not limited to Hepatitis C.

Hepatitis C

Hepatitis refers to acute or chronic disorders resulting from liver damage caused by viral, toxic, pharmacologic or immune-mediated factors. All forms of hepatitis share the pathologic features of hepatocellular necrosis and inflammatory cell infiltration of the liver. These changes to the liver may be manifested as an enlarged liver or an increase in the level of transaminase. The symptoms of acute viral hepatitis often appear suddenly and can include gastrointestinal abnormalities, darkened urine, jaundice and symptoms associated with reduced bile flow. Although chronic hepatitis is typically asymptomatic, and rarely causes major liver damage, cirrhosis and liver failure can occur as a result of some cases of chronic hepatitis.

One form of viral hepatitis, known as Hepatitis C, is caused by a flavivirus-like RNA agent. Hepatitis C virus can be identified as the causal agent of chronic or acute hepatitis by diagnostic tests that detect viral proteins or antibodies specific for the virus in the blood. Hepatitis C is a common cause of chronic hepatitis.

Hepatitis C virus (HCV) is a major cause of liver disease worldwide with an estimated occurrence of 150,000 to 170,000 new cases annually in the United States. Currently, it is estimated that about 3.9 million Americans have been infected with HCV. The leading cause of liver transplantation in adults is HCV, due to the damage it causes. HCV is transmitted primarily through inoculations and blood transfusions, although vertical transmission has also been documented. HCV has a high rate of progression (greater than 50%) to chronic disease and eventual cirrhosis. Chronic hepatitis C is characterized by several histological features in the liver which discriminate it from other forms of hepatitis, including bile duct damage, lymphoid follicles and fatty change.

Interferons are the only FDA-approved treatment for hepatitis C, and various types of interferons (e.g interferon-alpha) have been used clinically to treat HCV infections with varying degrees of success (Terranova et al., 1996, Control Clin Trials 17:123-129 and Montalto et al., 1998, Am J Gastroenterol., 93:950-953). It has also been found that two effective ribozymes (CR2 and CR4) can inhibit the expression of a cotransfected reporter gene containing HCV RNA target sequences (Welch et al., 1996, Gene Ther., 3:994-1001); and these results suggest that hairpin ribozymes may be useful for methods of treating HCV infection that involve gene therapy. Interferon treatment is characterized by low response rates and dose-limiting side effects. The effectiveness of interferon treatment has been improved by administering other agents such as thymosin alpha 1 in combination with interferon (Sherman et al., 1998, Hepatology, 27:1128-1135).

Chimpanzees and rodents have provided animal models for studying HCV infection in humans. Several features of human HCV infection are found in the chimpanzee model, including the frequency of persistent infection, and virus replication which occurs despite evidence of cellular and humoral immune responses (Walker et al., 1998, Springer Semin. Immunopathol., 19:85-98). However, although chimpanzees provide a useful model for studying HCV infection, they are not the most practical animals to work with. Efforts have therefore been made to develop useful rodent models for HCV.

According to one rodent model, 2-3 day old mice were infected intracerebrally with HCV (Deriabin et al., 1997, Vopr. Virusol.,42:251-253) and subsequently died 12-14 days later. Additionally, two independent transgenic mouse lines carrying the HCV core gene are now established. As these mice develop progressive hepatic stetosis, they provide a useful animal model for the study of pathogenesis in human HCV infection (Moriya et al., 1997, J. Gen. Verol., 78:1527). Another group has used a chimeric mouse model for the induction of hepatitis C viremia, using BNX (beige/nude/X-linked immunodeficient) mice preconditioned by total body irradiation and reconstituted with SCID mouse bone marrow cells. Following transplantation of HCV-infected liver fragments from patients with HCV-RNA-positive sera under the kidney capsule of the chimeric mice, viremia occurred in approximately 25% of these animals (Galun et al., 1995, J. Infect. Dis., 172:25-30).

### EXAMPLE 16

Treatment of an Infectious Disease with Recombinant Protein from Implanted Organized Tissue Constructs.

Postmitotic organoids genetically engineered to secrete therapeutic levels of recombinant interferon and/or thymosin alpha I are used to treat HCV infections. This can be accomplished initially in an animal model. Cells (e.g. myoblasts and fibroblasts) are isolated from animals and plated separately in tissue culture flasks. When the cells are nearly confluent they are harvested and plated at low density in tissue culture plates. The low density cultures are transduced with a retroviral vector containing the gene for recombinant interferon (Malaguarnera et al., 1998, Neuropsycholobiology, 37:94 and Tong et al., 1998, Cytokine Res., 2:81) as described in Example 2. Transduced cells are engineered into organized tissue constructs as described in Example 1. One or more recombinant interferon secreting organoids are implanted into the muscle bed overlying the fascia or in the peritoneal cavity under tension in animals anesthetized by inhalation of isoflurane or methozyflurane through a vaporizer and nosecone, (as described in Example 1). In vivo recombinant interferon tissue or serum levels are measured at varying times after implantation by standard radioimmunoassay or ELISA. It is expected that the increase in recombinant interferon levels in the implanted animals is adequate to treat the HCV infection. Successful treatment is measured as a decrease in the levels of HCV RNA (at least 5-10% and preferably 25-100%, as measured by the method of Northern blot analysis), by normalization of transaminase, and/or by an improvement in the histological pictures of test or infected animals as compared to normal animals. Serum aminotransferase (alanine transaminase and aspartate transaminase) are released from the acutely damaged hepatocytes and serum transaminase levels rise, often to levels exceeding 20-fold normal. Standard assays for determining transaminase levels are well known in the art and are used extensively clinically.

A given treatment for an infectious disease (e.g. Hepatitis C) according to the invention may be tested in an art accepted animal model of HCV by implanting the organized tissue producing a recombinant protein (e.g. interferon and/or thymosin alpha 1) into the diseased animal and observing clinical parameters over time. Art-accepted animal models of HCV include but are not limited to chimpanzee models that exhibit several features of human HCV infection, (Walker et al., supra), a rodent model wherein 2-3 day old mice are infected intracerebrally with HCV (Deriabin et al., supra), transgenic mouse lines carrying the HCV core gene and thereby providing a useful animal model for the study of pathogenesis in human HCV infection (Moriya et al., supra) and a chimeric mouse model transplanted with HCV-infected liver fragments from patients with HCV-RNA-positive sera (Galun et al., supra).

A chimeric mouse model was used for the induction of hepatitis C viremia, using BNX (beige/nude/X-linked immunodeficient) mice preconditioned by total body irradiation and reconstituted with SCID mouse bone marrow cells. HCV-infected liver fragments from patients with HCV RNA-positive sera were transplanted under the kidney capsule of the chimeric mice (Galun et al., supra).

Therapeutic efficacy of treatment of HCV according to the invention by implantation of an organized tissue producing interferon and/or thymosin alpha 1 as described herein, is indicated by changes in clinical parameters such as changes in HCV RNA levels, and changes in transaminase levels (by at least 5-10% and preferably 25-100%).

Human HCV patients may be treated accordingly by implanting organoids producing interferon and/or thymosin alpha 1, measuring the level of these recombinant proteins, determining changes in HCV RNA levels, and changes in transaminase levels, and the amelioration of symptoms associated with HCV over time.

H. Muscle Wasting and Whole Body Wasting Disorders

The invention also provides methods of treating muscle wasting and whole body wasting disorders.

Muscle Wasting

Muscle wasting is a loss of muscle mass due to reduced protein synthesis and/or accelerated breakdown of muscle proteins, largely as a result of activation of the non-lysosomal ATP-ubiquitin-dependent pathway of protein degradation. Muscle wasting is caused by a variety of conditions including cachexia associated with diseases including various types of cancer and AIDS, febrile infection, denervation atrophy, steroid therapy, surgery, trauma and any event or condition resulting in a negative nitrogen balance. Muscle wasting also occurs following nerve injury, fasting, fever, acidosis and certain endocrinopathies. Muscle wasting can be detected by measuring protein synthesis and or degradation, the level of production of cell damage markers such as creatine kinase, the activity of a heat shock protein promoter, and changes in the level of components of the ubiquitin dependent protein degradation pathway.

Patients with catabolic wasting disease (e.g. cancer cachexia) are in negative nitrogen balance and suffer a significant and life threatening weight loss. Cancer cachexia is characterized by weakness, anorexia, anemia and progressive skeletal muscle wasting. Other causes of wasting are severe burns, trauma, and major surgery. Wasting diseases effect the quality of life, and are associated with a poor response to chemotherapy as well as decreased survival time following chemotherapy (Tamura et al., 1995, Clinical CancerResearch, 1:1353-1358, Bartlett et al., 1994, Cancer, 73:1499-1504, Tisdale, 1997, Journal of National Cancer Institute, 89: 1763-1773). It is currently hypothesized that the mechanism responsible for the development of cancer cachexia involves production of inflammatory cytokines, which in turn orchestrate a series of complex interrelated steps that ultimately lead to a chronic state of wasting, malnourishment, and death. In an animal model of catabolic wasting diseases, Lewis/Wistar rats are subcutaneously inoculated with the MAC-33 tumor, a spontaneously metastasizing mammary adenocarcinoma. The metastasis of the MAC-33 tumor causes weight loss in the rat and ultimate death. Treatment of these rats with growth hormone, insulin and/or somatostatin resulted in increased body weight and muscle size, as compared to control animals that experienced weight loss over the same period (Bartlett et al.,supra).

In vitro Production of a Skeletal Muscle Organoid Having in vivo-like Gross and Cellular Morphology

Using an apparatus and method as generally described above, a skeletal muscle organoid having an in vivo-like gross and cellular morphology was produced in vitro. An overview of the stages of skeletal muscle growth and regeneration is shown in FIG. 6. As shown, during skeletal muscle development embryonic myoblasts proliferate, differentiate, and then fuse to form multi-nucleated myofibers. Although the myofibers are non-proliferative, a population of muscle stem cells (i.e., satellite cells), derived from the embryonic myoblast precursor cells, retain their proliferative capacity and serve as a source of myoblasts for muscle regeneration in the adult organism. Therefore, either embryonic myoblasts or adult skeletal muscle stem cells may serve as one of the types of precursor cells for in vitro production of a skeletal muscle organoid.

To produce skeletal muscle cells capable of secreting a bioactive compound, primary rat or avian cells or immortalized murine cells secreting recombinant human growth hormone, were suspended in a solution of collagen and Matrigel™ which was maintained at 4° C. to prevent gelling. The cell suspension was then placed in a semi-cylindrical vessel with tissue attachment surfaces coupled to an interior surface at each end of the vessel. The vessel was positioned in the bottom of a standard cell culture chamber. Following two to four hours of incubation at 37° C., the gelled cell suspension was covered with fresh culture medium (renewed at 24 to 72 hour intervals) and the chamber containing the suspended cells was maintained in a humidified 5% CO₂ incubator at 37° C. throughout the experiment.

Between the second and sixth day of culture, the cells were found to be organized to the extent that they spontaneously detached from the vessel. At this stage, the cells were suspended in culture medium while coupled under tension between tissue attachment surfaces positioned at either end of the culture vessel. During the subsequent ten to fourteen days, the cells formed an organoid containing skeletal myofibers aligned parallel to each other in three dimensions. The alignment of the myofibers and the gross and cellular morphology of the organoid were similar to that of in vivo skeletal muscle.

To carry out the above method, an apparatus for organoid formation was constructed from silastic tubing and either VELCRO™ or metal screens as follows. A section of silastic tubing (approximately 5 mm I.D., 8 mm O.D., and 30 mm length) was split in half with a razor blade and sealed at each end with silicone rubber caulking. Strips of VELCRO™ (loop or hook side, 3 mm wide by 4 mm long) or L-shaped strips of stainless steel screen (3 mm wide by 4 mm long by 4 mm high) were then attached with silicone rubber caulking to the interior surface of the split tubing near the sealed ends. The apparatus was thoroughly rinsed with distilled/deionized water and subjected to gas sterilization.

Skeletal muscle organoids were produced in vitro from a C2C12 mouse skeletal muscle myoblast cell line stably co-transfected with recombinant human growth hormone-expressing and β-galactosidase-expressing (β-gal) constructs. Dhawan et al., 1991, Science 254:1509-1512. Cells were plated in the vessel at a density of 1-4×10⁶ cells per vessel in 400 μl of a solution containing extracellular matrix components. The suspension of cells and extracellular matrix components was achieved by the following method. The solution includes 1 part Matrigel™ (Collaborative Research, Catalog No. 40234) and 6 parts of a 1.6 mg/ml solution of rat tail Type I collagen (Collaborative Research, Catalog No. 40236). The Matrigel was defrosted slowly on ice and kept chilled until use. The collagen solution was prepared just prior to cell plating by adding to lyophilized collagen, growth medium (see constituents below), and 0.1N NaOH in volumes equivalent to 90% and 10%, respectively, of the volume required to obtain a final concentration of 1.6 mg/ml and a pH of 7.0-7.3. The collagen, sodium hydroxide and growth medium were maintained on ice prior to and after mixing by inversion.

Freshly centrifuged cells were suspended in the collagen solution by trituration with a chilled sterile pipet. Matrigel™ was subsequently added with a chilled pipet and the suspension was once again mixed by trituration. The suspension of cells and extracellular matrix components was maintained on ice until it was plated in the vessel using chilled pipet tips. The solution was pipetted and spread along the length of the vessel, taking care to integrate the solution into the tissue attachment surfaces. The culture chamber containing the vessel was then placed in a standard cell culture incubator, taking care not to shake or disturb the suspension. The suspension was allowed to gel, and after 2 hours the culture chamber was filled with growth medium such that the vessel was submerged.

For a period of three days the cells were maintained on growth medium containing DMEM-high glucose (GIBCO-BRL), 5% fetal calf serum (Hyclone Laboratories), and 1% penicillin/streptomycin solution (final concentration 100 units/ml and 0.1 μg/ml, respectively). On the fourth day of culture, the cells were switched to fusion medium containing DMEM-high glucose, 2% horse serum (Hyclone Laboratories), and 100 units/ml penicillin for a period of 4 days. On the eighth day of culture, the cells were switched to maintenance medium containing DMEM-high glucose, 10% horse serum, 5% fetal calf serum, and 100 units/ml penicillin for the remainder of the experiment. Before the organoids were ready for implantation, some were cultured in maintenance media containing 1 μg/ml of cytosine arabinoside for the final four to eight days. Treatment with cytosine arabinoside eliminated proliferating cells and produced organoids including substantially post-mitotic cells.

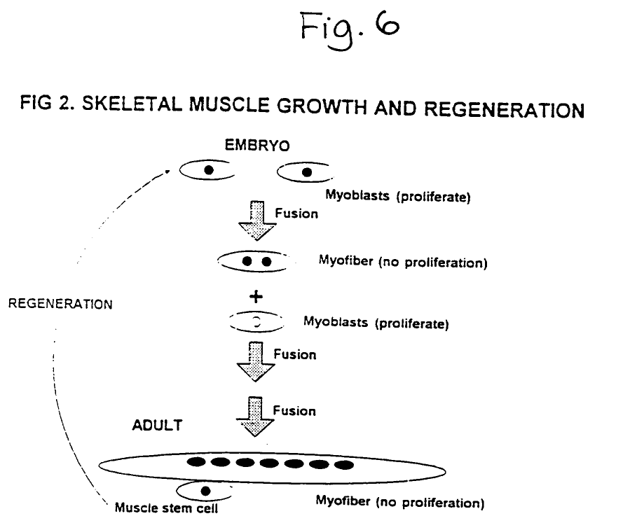

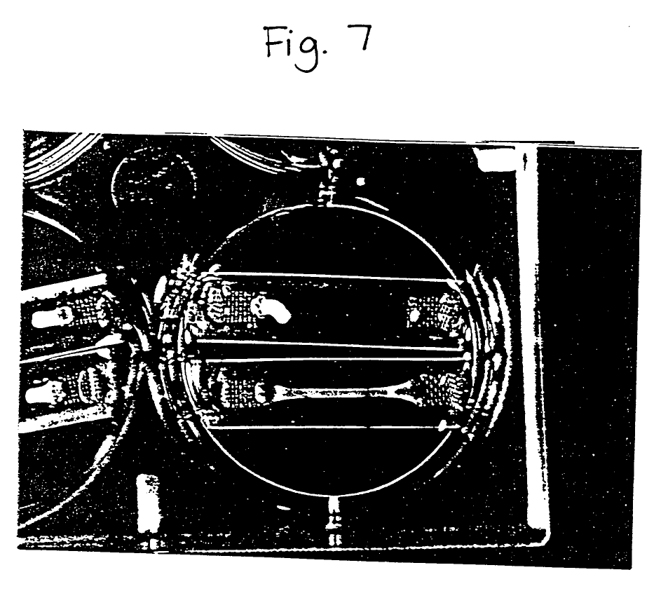

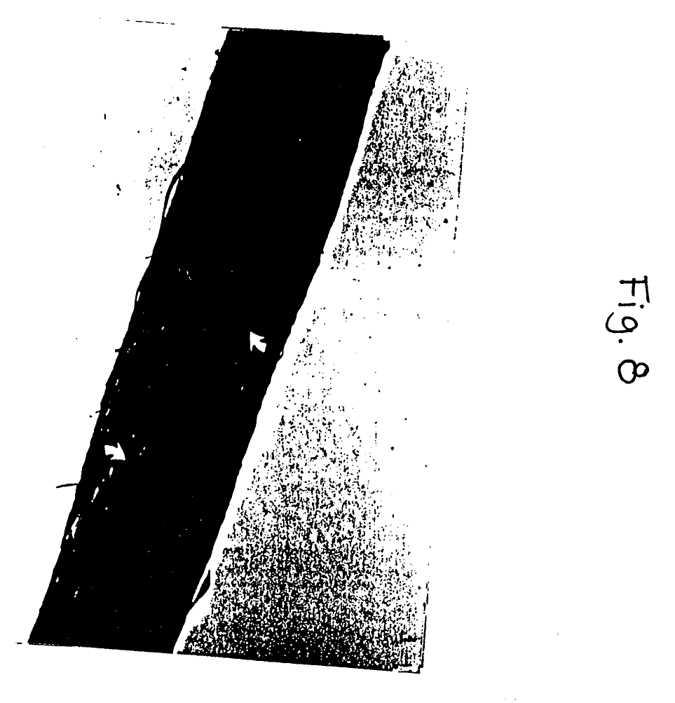

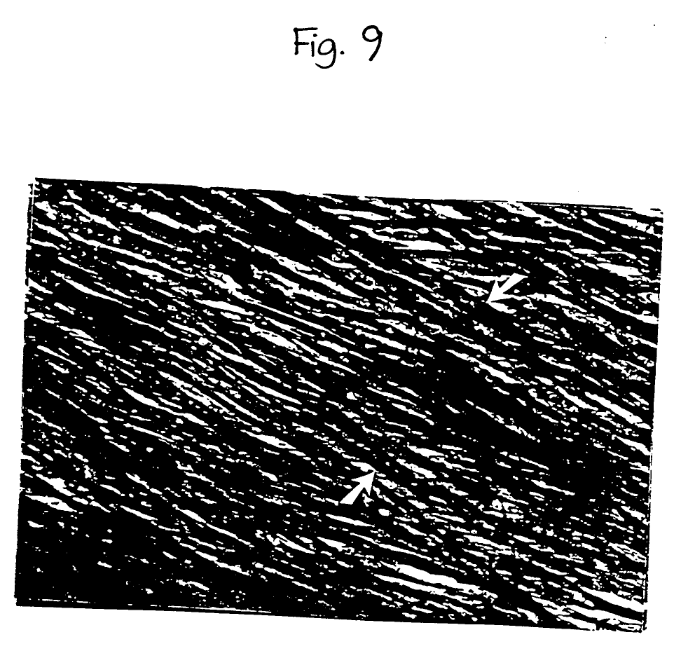

The cell-extracellular matrix gel (cell-gel) formed in vitro from these stably transfected C2C 12 cells 48 hours after plating are shown in FIG. 7. In the upper half of the figure the cell-gel has detached from one of the tissue attachment surfaces. The resultant contraction demonstrates the tension developed in the gel between the tissue attachment surfaces. FIGS. 8 and 9 demonstrate the presence of a muscle-specific contractile protein (i.e., brown staining following incubation with an antibody to sarcomeric tropomyosin), in parallel arrays of highly organized and longitudinally oriented myofibers in mammalian skeletal muscle organoids following three weeks of culturing in the apparatus shown in FIG. 1. FIG. 8 represents a middle section of a 3 week old mammalian C2C12 muscle cell organoid stained for sarcomeric tropomyosin, showing longitudinally oriented myofibers (arrows). Magnification is approximately 40×. FIG. 9 shows parallel aligned myofibers (arrows) on the surface of a 3 week old mammalian C2C 12 muscle cell organoid stained for sarcomeric tropomyosin. Magnification is approximately 400×. Moreover, FIG. 14B shows the retention of myofiber organization following organoid implantation.

### EXAMPLE 17

Delivery of Human Growth Hormone to Mice by Implanting Skeletal Muscle Organoids

FIG. 11 shows an overview and comparison of myoblast and myofiber gene therapy. Both methods generally involve isolating myoblasts from a patient in need of gene therapy, inserting into the myoblasts a DNA sequence encoding a bioactive compound, and expanding the myoblast cell population by in vitro culturing. In contrast to myoblast gene therapy, the myoblasts used in myofiber gene therapy are further cultured in vitro under conditions which result in the formation of an organoid having in vivo-like gross and cellular morphology. The organoid is subsequently implanted into the patient to deliver the bioactive compound.

To carry out the delivery of a bioactive compound to an organism, skeletal muscle organoids were formed in vitro, as described above, from C2C12 mouse skeletal muscle myoblasts stably co-transfected with recombinant human growth hormone-expressing and β-galactosidase-expressing constructs. Prior to implantation, in vitro production of recombinant human growth hormone (“rhGH”) was measured by radioimmunoassay according to the manufacturer's instructions (Nichols Institute Diagnostics, San Juan Capistrano, Calif.). Between three and twenty-four days of culture, the mean rhGH production ranged between 1.0 and 3.5 μg/day/organoid (see Table 1).

TABLE 1
IN VITRO PREIMPLANT SUMMARY

| | | Initial | | | | |
|---|---|---|---|---|---|---|
| | | Cell # | Age | Mean rhGH | | |
| | | per or- | of or- | (μg/day/ | | Treatment |
| | | ganoid | ganoid | organoid) | | of |
| Experiment | Date | (× 10⁴) | (Days) | (N =) | | organoids |
| IMPLANT 1 | 8/24 | 6 | 3 | 1.9 | (2) | none |
| | | | 7 | 3.5 | (2) | |
| IMPLANT 2 | 9/21 | — | — | — | | — |
| IMPLANT 3 | 10/5 | 4 | 7 | 1.7-2.8 | (7) | none |
| | | | 12 | 1.9-2.5 | (6) | |
| IMPLANT 4 | 10/20 | 2 | 21 | 2.2-2.6 | (5) | none |
| IMPLANT 5 | 10/25 | 2 | 12 | 2.9 | (12) | no cyto- |
| | | | 12 | 2.0 | (4) | sine ara- |
| | | | | | | binoside |
| | | | | | | (“araC”) |
| | | | | | | 1 ug/ml |
| | | | | | | araC for |
| | | | | | | 4 days |
| IMPLANT 6 | 11/8 | 3 | 19 | 1.0 | (6) | no araC |
| | | | | 1.0 | (6) | 1 ug/ml |
| | | | | | | araC for |
| | | | | | | 5 days |
| IMPLANT 7 | 11/9 | 3 | 17 | 0 | (3) | control |
| | | (non- | | | | exper- |
| | | rhGH | | | | iment |
| | | secret- | | | | |
| | | ing) | | | | |
| IMPLANT 8 | 11/3 | 2 | 14-20 | 1.5 to 2.2 | (6) | no araC |
| | | | | 1.2 to 1.6 | (6) | 1 ug/ml |
| | | | | | | araC for |
| | | | | | | 5 days |
| IMPLANT 9 | 11/30 | 1-2 | 24 | 1.7 to 2.4 | (8) | 1 ug/ml |
| | | | | | | araC for |
| | | | | | | 8 days |
| IMPLANT 10 | 12/5 | 1.5-2.0 | 20 | 2.1 to 2.9 | (14) | 1 ug/ml |
| | | | | | | araC for |
| | | | | | | 4 days |

The organoids were implanted into adult C3HeB/FeJ mice (i.e., syngeneic to C2C12 cells) by the following method. Mice were weighed to determine dosages of cyclosporine and anesthetic. One hour prior to the surgical implantation of the organoid, each mouse was given an injection of 60 mg/kg of cyclosporine A. Each mouse was then selected in turn and anesthetized by intramuscular injection of 55 mg/kg Ketamine, 1 mg/kg Promazine, and 5 mg/kg Xylazine. The site of implantation was then depilatated with Nair™ or by shaving, and prepped for aseptic surgery. For organoids implanted subcutaneously, a four to six centimeter long incision was made along the back, the organoid was implanted in either a free floating state or fixed under tension (e.g., attached to the tissue attachment surfaces), and the incision was closed with four to six sutures of 4.0-black silk.

For organoids implanted intramuscularly, a 15 to 30 millimeter incision was made parallel to the anterior tibialis muscle (e.g., anteriolateral aspect of the lower hind limb) to provide access to the muscle sheath. The anterior tibialis was gently split with forceps from tendon to tendon parallel to the muscle belly, thus providing a cavity for insertion of the organoid. The organoid was carefully removed from the vessel by prying the ends off the tissue attachment surfaces with sterile forceps and inserting it, under resting tension, in the implantation site. The incision was closed as described above. Mice were then followed post-surgically for distress and upon regaining consciousness were returned to a skeletal care facility. Cyclosporine injections are repeated daily for the duration of the experiment. The experimental protocol for the implantation of skeletal muscle organoids is summarized in Table 2 below.

TABLE 2
IN VIVO PROTOCOL SUMMARY

| | | | # of | rhGH Producers |
|---|---|---|---|---|
| | | Site of | Surviving | (# and method |
| Experiment | Date | Implant | Skeletals | of implant) |
| IMPLANT 1 | 8/24 | intramuscular | 2 of 2 | 0 (1 free) |
| | | free-floating | | |
| IMPLANT 2 | 9/21 | controls only - | 6 of 6 | no organoids |
| | | cyclosporine | | implanted |
| | | dose-response | | |
| IMPLANT 3 | 10/5 | subcutaneous | 3 of 4 | 1 (3C - 2 free) |
| | | free-floating | | |
| IMPLANT 4 | 10/20 | subcutaneous | 2 of 3 | 2 (2D - 2 fixed) |
| | | fixed under | | (3D - 1 fixed/1 free) |
| | | tension | | |
| IMPLANT 5 | 10/25 | subcutaneous | 1 of 2 | 1 (1E - 3 fixed) |
| | | fixed under | | |
| | | tension | | |
| IMPLANT 6 | 11/8 | subcutaneous | 4 of 7 | 3 (6A, 6D, 6E - 1 |
| | | fixed under | | fixed) |
| | | tension | | (6G - no organoid) |
| IMPLANT 7 | 11/9 | subcutaneous | 2 of 3 | 0 (7A and 7C - 1 |
| | | fixed under | | fixed, non-rhGH |
| | | tension | | secreting organoid) |
| IMPLANT 8 | 11/13 | subcutaneous | 5 of 8 | 4 (8C, 8D, 8F and |
| | | fixed under | | 8G - 1 fixed) |
| | | tension | | |
| IMPLANT 9 | 11/30 | subcutaneous | 7 of 7 | 5 (9A, 9B, 9C, 9D |
| | | fixed under | | and 9F - 1 fixed) |
| | | tension or | | 1 (9E - 1 free) |
| | | free-floating | | (9G - no organoid) |
| IMPLANT 10 | 12/5 | subcutaneous | 7 of 11 | 7 (10A, 10B, 10C, |
| | | fixed under | | 10D, 10F, 10G, and |
| | | tension | | 10J - 1 fixed) |

Blood was collected every one to seven days by tail bleeding from the mice. Sera concentrations of rhGH were measured by radioimmunoassay according to the manufacturer's instructions (Nichols Institute Diagnostics, San Juan Capistrano, Calif.).

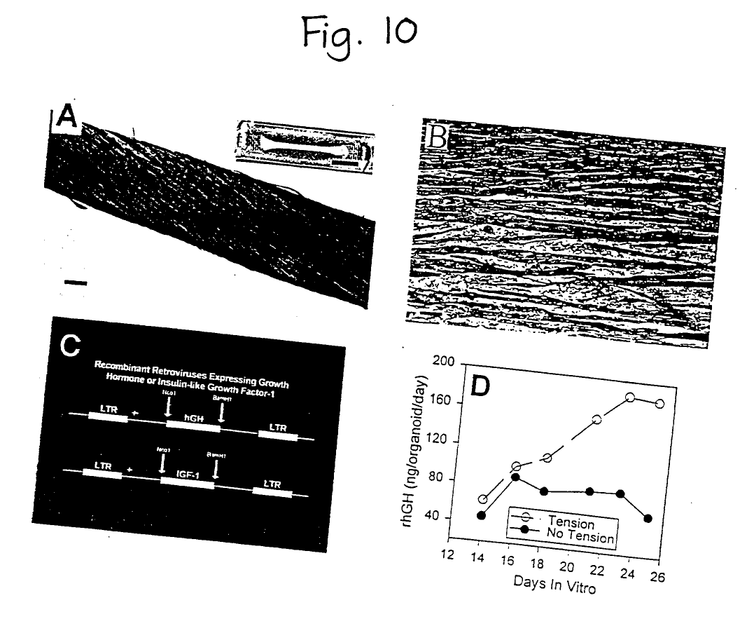

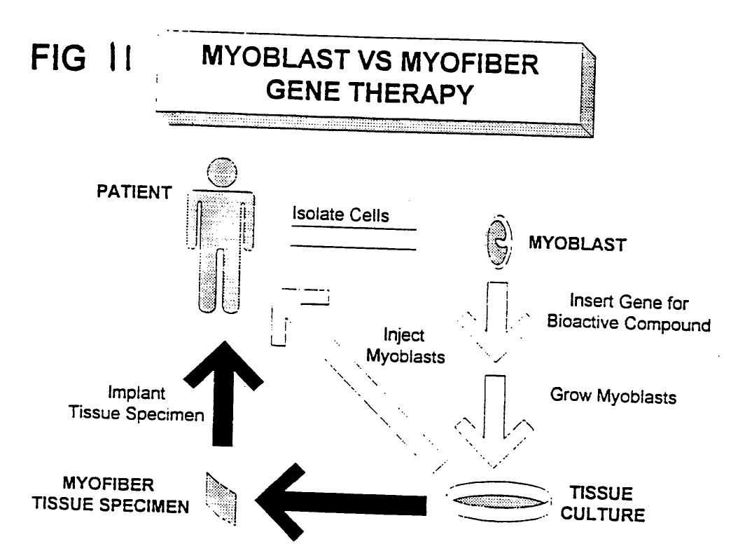

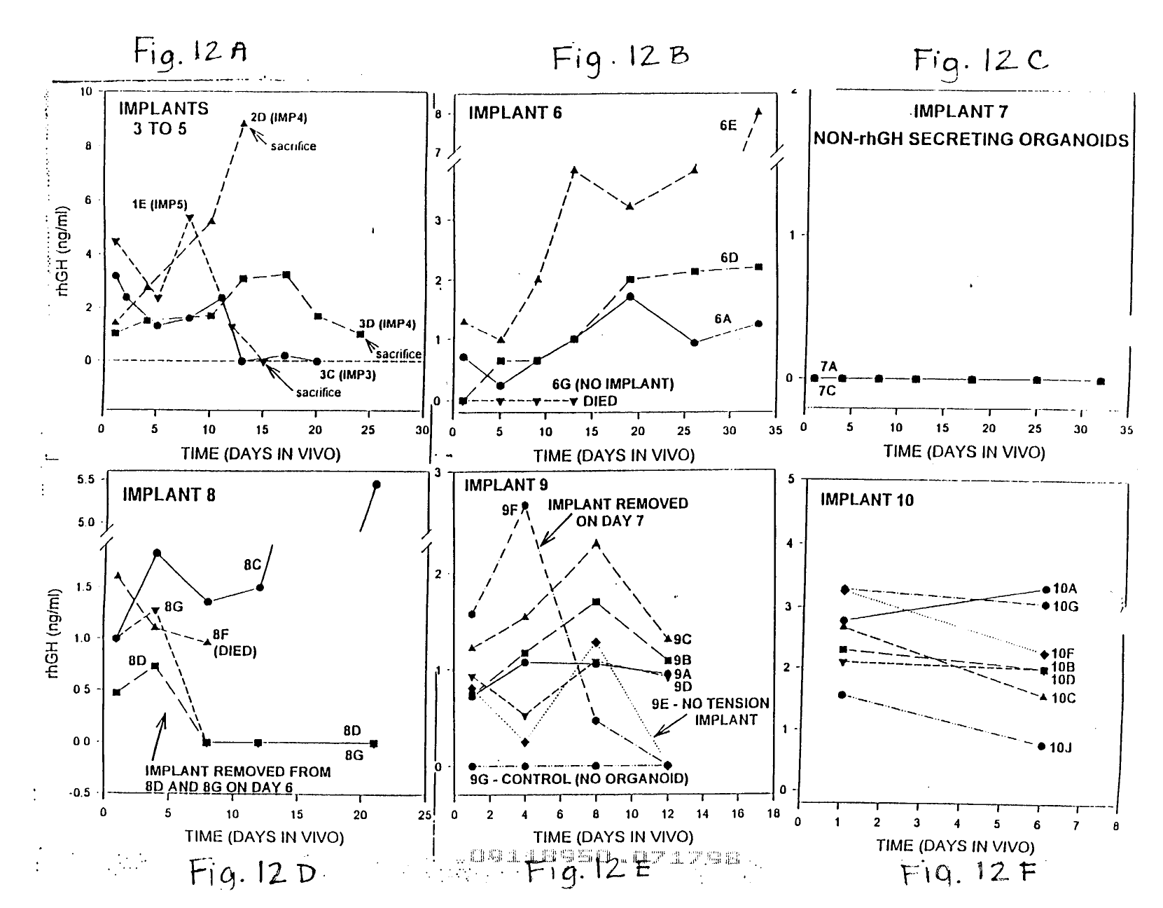

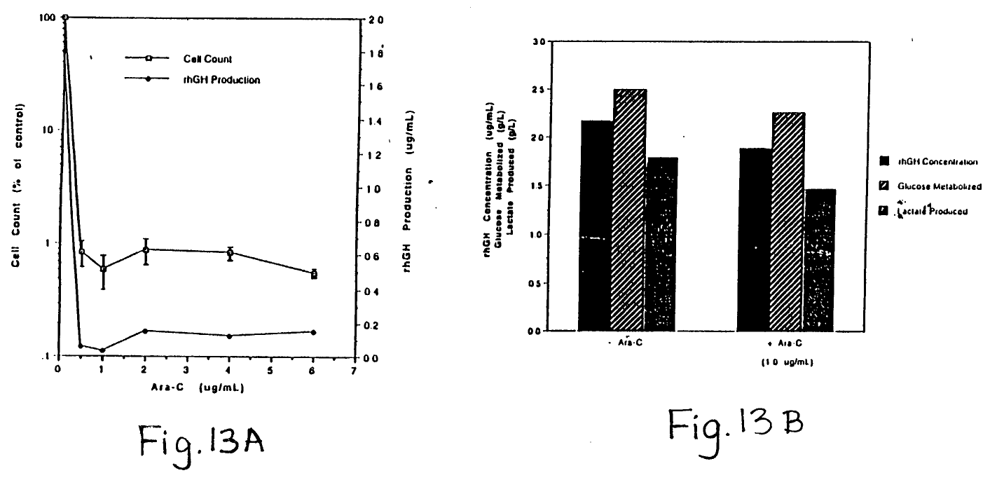

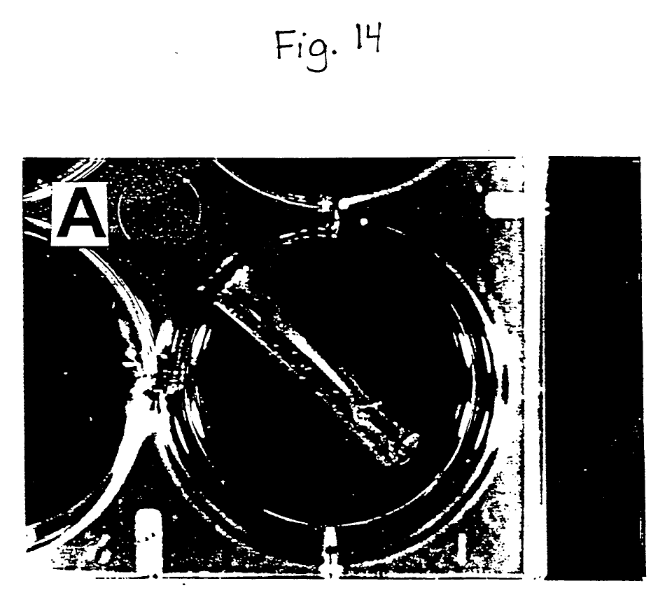

As shown in FIGS. 12A-12F, rhGH was detected in the blood of skeletals receiving rhGH organoid implants, but not in controls (6G, 7A, 7C, and 9G) for up to thirty-three days post-implantation. Serum concentrations were elevated as high as approximately 5.5 to 9 ng/ml in skeletals receiving multiple implants of rhGH producing organoids (1E, 2D), whereas serum from skeletals receiving no implant (6G, 9G) or implants of non-rhGH secreting organoids (8A and 8C) contained no detectable rhGH. In addition, skeletals receiving organoids treated in vitro with cytosine arabinoside prior to implantation (1 E, 6E, 8D, 8F, 8G, 9A through 9F, and 10A through 10J) demonstrated serum rhGH levels comparable to those of skeletals receiving implants which were not treated in vitro with cytosine arabinoside prior to implantation (i.e., 2D, 3C, 3D, 6A, 6D, and 8C). Under the conditions used in this study, cytosine arabinoside treatment kills greater than 99% of proliferating C2C 12 myoblasts while having only a minor effect on myofiber metabolism and rhGH secretion (FIG. 13). Moreover, FIG. 14C shows that the rhGH gene and the β-galactosidase gene are only expressed in post-mitotic myofibers. These results demonstrate that organoids including substantially post-mitotic cells can deliver therapeutic levels of a bioactive compound for up to thirty-three days post-implantation.

FIG. 14(A) rhGH secreting muscle organoid removed after 2 weeks in mouse 2D; (B) H&E stained cryostat cross-section of organoid shown in (A), with well differentiated myofibers running longitudinally in the organoid, and parallel to each other (arrows); and (C) X-gal blue staining of β-galactosidase activity in the cells containing the rhGH gene and β-gal gene (co-transfected in the same C2C12 myoblasts).

It is noteworthy that within forty-eight hours following the removal of implants (i.e., 8D, 8G and 9F), rhGH was undetectable in the sera of skeletals previously having serum concentrations as high as 2.6 ng/ml. These data demonstrate the reversibility of delivering bioactive compounds by this method. In addition, organoids removed from skeletals may be re-incubated in vitro (see e.g., FIG. 14A). For example, the two organoids implanted into skeletal 3D produced 188 ng/day of rhGH in vitro post-implantation. These data suggest the feasibility of removing organoids and subsequently reimplanting them such that bioactive compounds may be delivered during multiple treatment periods separated in time. Moreover, the data suggest the feasibility of transplanting sequentially, at different sites within the same organism, organoids functioning as paracrine organs.

The rhGH production of 188 ng/day in vitro by organoids from skeletal 3D and the in vivo serum levels of 1.0 ng/ml on day twenty-four (i.e., just prior to removal) suggest a 188-fold difference between organoid production and steady state circulating levels of rhGH in the skeletal. These results compare favorably to the 500-fold difference between rhGH concentrations delivered by direct subcutaneous injection and steady state circulating levels, (Yang et al., 1995, Circulation 92:262-267, (1000 μg/day rhGH by direct subcutaneous injection produced 2 μg/ml serum concentrations in rats). It is also noteworthy that the organoid maintained in vivo under tension produced approximately 144 ng/ml when placed in vitro on removal from the skeletal, while the free floating organoid produced only 40 ng/ml when placed in vitro on removal from the skeletal. In addition, an organoid implanted under no tension (9E) was a poorer producer of rhGH in vivo than those placed under tension (9A, 9B, 9C). These results suggest that maintaining organoids under tension enhances the production and delivery of bioactive compounds.

### EXAMPLE 18

rhGH Secreted from C2-organoids is Biologically Active and can Attenuate Muscle Atrophy in Hindlimb-unloaded Host Skeletal Muscle in vivo.

Organized tissue constructs genetically engineered to secrete growth hormone were used to treat muscle wasting in a hindlimb suspension model of skeletal muscle wasting.

Murine C2C 12 skeletal myoblasts stably transduced with the gene for rhGH under control of the retroviral LTR promoter (Dhawan et al., 1991, Science, 254:1509-1512) using retroviral vectors were tissue engineered into implantable C2-organoids secreting pharmacological levels of rhGH in vitro, as described herein. The C2-organoids were subsequently treated with cytosine arabinoside to remove unfused proliferating myoblasts. These organized tissue constructs secreted 3-5 μg rhGH/day in vitro (data not shown). When implanted subcutaneously under tension into syngeneic C3HeB/FeJ mice, rapid and stable appearance of physiological levels of rhGH in the serum occurred for greater than 12 weeks. The implanted C2-organoids are well vascularized by the host, and retain their preimplantation structure, allowing surgical removal. Removal of the implants leads to the rapid disappearance of rhGH from the sera. The rhGH released from the C2-organoids is biologically active, based on the down regulation of a GH-sensitive 20kD protein made in the liver, and secreted as a major urinary protein [MUP] (Vandenburgh et al, 1998, Human Gene Therapy, In Press).

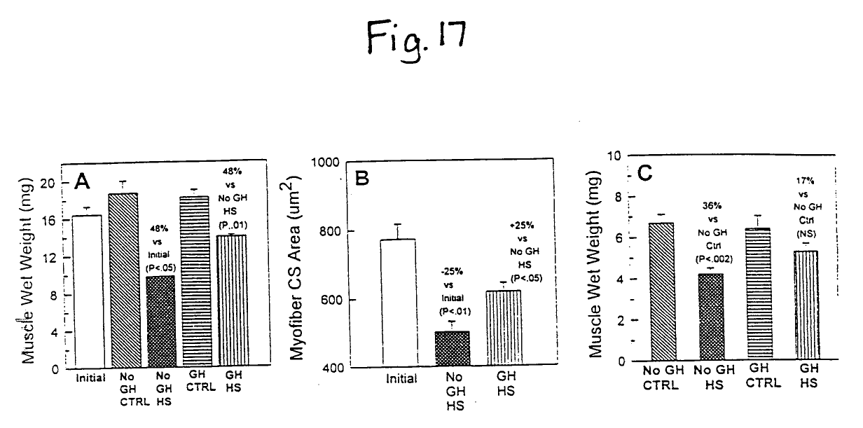

Animals implanted with rhGH secreting C2-organoids show a significant down regulation of MUP protein levels which lasts as long as the implant remains in the animal (FIG. 16A, 16B). Removal of the implant leads to a return of MUP to preimplantation levels (data not shown). Organoids are thus effective for the long-term delivery of biologically active proteins such as rhGH. FIG. 16 demonstrates that rhGH secreted from muscle organoids is biologically active.

We have tested whether acute muscle wasting in a hindlimb unloaded mouse model can be reduced by rhGH-secreting C2-organoid implants (Vandenburgh et al, 1998, supra). Initial studies were performed with the plantaris muscle since it is more growth hormone sensitive than the soleus (Aroniadou-Anderjaska et al., 1996, Tissue Cell 28:719-724; Grindeland et al., 1994, Am. J. Physiol. Regul. Interg. Comp. Physiol. 267:R316-R322).

Skeletal muscle disuse atrophy was induced in mice by hindlimb unloading (HU) according to the following method. A headdown suspension or tail cast suspension is a widely accepted model for skeletal muscle disuse atrophy (Wronski et al., 1987, Aviat. Space Environ. Med., 58:63-68, Park et al., 1993, Aviat. Space Environ. Med., 64: 401-404). Animals were suspended in individual cages using traction tape on the tail. Their forelimbs remained in contact with the ground while their hindlimbs were freely suspended. Six to fourteen days of suspension induces significant hindlimb muscle atrophy in mice (Haida et al., 1989, Exptl. Neurol., 103: 68-76) and rats (Morey-Holton, 1981, Physiologist, 24 (Suppl.):S45-S48). Hindlimb unloading caused the fast plantaris and slow soleus muscles to atrophy by 21% to 35% (P<0.02). Transduced C2C12 skeletal myoblasts were implanted in HU mice as described in Example 1. Following implantation of phGH secreting organized tissue constructs, muscle weight and myofiber cross sectional areas increased significantly in both the plantaris (41% and 68%, respectively, P<0.05) and soleus muscles (55% and 22%, respectively, P<0.05) as compared to HU animals implanted with non-rhGH secreting organized tissue constructs. Furthermore, muscle atrophy was not attenuated in mice receiving daily injections of purified rhGH (lmg/kg/day).

Based on both muscle wet weight (FIG. 17A) and myofiber cross sectional area (FIG. 17B), animals implanted with rhGH-secreting C2-organoids show significant attenuation of muscle wasting over a 6 day period compared to animals implanted with control, non-rhGH-secreting C2-organoids. Similar results have also been obtained in additional experiments with the less GH-sensitive soleus muscle (FIG. 17C). These studies support therapeutic efficacy since injected rhGH has been found to be effective in attenuating rat muscle wasting only in combination with moderate exercise. Delivery of continuously synthesized rhGH according to the invention may thus be more effective than daily rhGH injections since GH has a half life of less than 10 min in the circulation. In FIG. 17, six to eight week old C3HeB/FeJ mice were implanted with 2-3 C2-organoids per animal engineered from either normal C2C12 myoblasts or growth hormone (GH)-secreting C2C12 myoblasts. Each rhGH-secreting C2-organoid produced 1 to 3 μg rhGH per day preimplantation and a steady state serum level of 2-3 ng/ml from Day 1 to Day 8 after implantation. On Day 1 to 3 after implantation, half of the animals were hindlimb suspended (HS) for 5-8 days (n=3 to 6 per group). Hindlimb muscles were processed for wet weight and myofiber cross-sectional areas by standard protocols. (A) and (B) are data for the plantaris muscle while (C) is data for the soleus muscle. Each value is the mean ±SE of 3 to 6 animals and statistical analyses by unpaired t-tests.

Therapeutic efficacy of treatment of skeletal muscle wasting in a hindlimb suspension animal model of skeletal wasting was measured by measuring muscle weight and myofiber cross sectional areas in plantaris and soleus muscles.

Human patients suffering from skeletal muscle wasting may be treated accordingly by implanting organoids producing growth hormone, measuring the level of growth hormone, determining changes in muscle weight and myofiber cross sectional areas in plantaris and soleus muscles, and the attenuation of symptoms associated with muscle wasting over time.

### EXAMPLE 19

Primary Rat Neonatal Myoblast Tissue Engineered into Organoids

Primary Fisher 344 neonatal myoblasts organoids were recently engineered to release physiological levels of rhGH when transduced with a replication defective retroviral MFG-hGH expression vector (FIG. 10C).

Myofiber tension is an important regulator of rhGH secretion in these R-organoids (FIG. 10D), as described herein for C2 organoids.

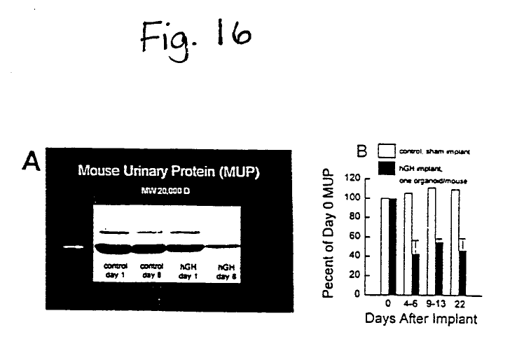

Adult rat myoblast isolation, rhX gene transduction, and organoid formation are performed as follows. Primary myoblasts were isolated by standard isolation procedures (Cantini et al., 1994, In Vitro Cell Dev. Biol., 30A: 131-133) from the tibialis anterior muscle of adult 120-150g rats. Approximately 1×10⁶ cells were isolated from one tibialis anterior muscle and expanded to 14×10⁶ cells in 12-14 days. Twenty-five percent confluent myoblast cultures in T75 flasks were transduced with the MFG-hGH retroviral expression vector (FIG. 10C). When confluent, the transduced myoblasts were subcultured at a density of 100,000 cells/well and allowed to differentiate into myofibers. Cultures secreted 600-900 ng rhGH/10⁶ cells/day (FIG. 15) a level comparable to the C2-organoids which were biologically active when implanted in adult mice (Vandenburgh et al., 1996, Human Gene Therapy, 7:2195). R-organoids were also formed from these cells and maintained in vitro for 2-3 weeks. Adult rat myoblasts thus behave in a similar fashion to the neonatal rat myoblasts. The adult myoblast preparations have a significantly lower initial yield of cells per experiment (1 vs 100×10⁶), and therefore a time period of approximately several extra weeks is necessary if adult cells are used. In FIG. 15, myoblasts were isolated from the tibialis anterior muscle of adult rats and transduced with the MFG-hGH retroviral expression vector. After differentiation into myofibers, medium samples were removed, diluted 1:50, and assayed for rhGH by RIA. Each point is the mean ±S.E. (N=4).

### EXAMPLE 20

Delivery of rHGH According to the Invention is more Effective than Daily rHGH Injections in the Prevention of the Hindlimb Unloaded Atrophy of the Slow Soleus Muscle.

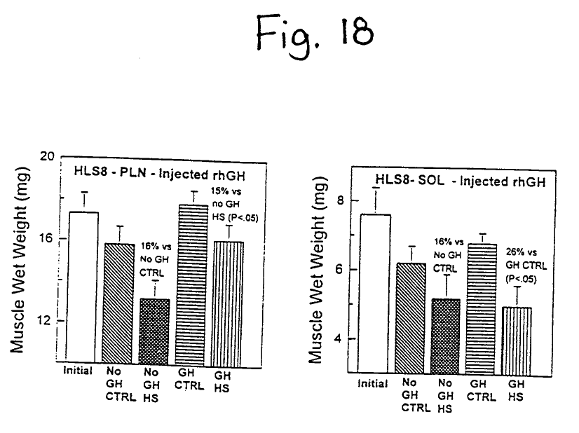

We injected purified rhGH (Genentech) daily to determine its ability to attenuate hindlimb unloaded muscle atrophy in mice. Unlike the results of others in rats indicating that injected rhGH alone could not attenuate hindlimb unloading-induced muscle atrophy (Grindeland et al., 1994, Am. J. Physiol. Regul. Integr. Comp. Physiol. 267:R316-R322; Linderman et al., 1994, Am. J Physiol. Integr. Comp. Physiol. 267:R365-R37; Roy et al., 1996, J. Appl. Physiol. 81:302-311), we found in the mouse model that injected rhGH was effective in attenuating atrophy of the fast plantaris muscle (FIG. 18A), but not the slow soleus muscle (FIG. 18B). This may be due to the fact that slow muscles are less sensitive to the anabolic effects of rhGH than fast muscles (Aroniadou-Anderjaska et al., 1996, Tissue Cell 28:719-724; Grindeland et al., 1994, Am. J. Physiol. Regul. Integr. Comp. Physiol. 267:R316-R322). In contrast, rhGH-secreting C2-organoids were equally as effective in attenuating hindlimb unloaded muscle atrophy in both the fast plantaris and slow soleus muscles (FIG. 17A versus 17C). These results support the hypothesis that delivery of rhGH according to the invention is more effective than daily rhGH injections in treating atrophy of skeletal muscles. In FIG. 18, the experiments were performed in an identical fashion to those described above for FIG. 17, except that the animals were not implanted with C2-organoids but were injected daily with rhGH (1 mg/kg bodyweight) starting one day before hindlimb unloading. Each value is the mean ±S.E. of 3-6 animals and statistical analyses by unpaired t-tests.

### EXAMPLE 21

Delivery of Bone Morphogenetic Protein to an Organism by Implanting Skeletal Muscle Organoids

1. Transduction and Selection of C2C12 Myoblasts Expressing rhBMP-6

φ2 packaging cells producing high titers (>1×10⁷ pfu) of retrovirus containing the pLX(rhBMP-6)SN expression vector were provided by Dr. Vladimir Drozdoff, Department of Medicine, Vanderbilt University. Myoblast cell cultures, 50% confluent in T-75 flasks, were incubated for eight hours in 20 ml of conditioned media from the high viral titer packaging cells. The media was supplemented with 4 μg/ml of polybreen. After eight hours, the cells were placed in DMEM+10% fetal calf serum containing 2 μg/ml of polybreen, and cultured for an additional 48-72 hr, or until the cells had undergone one or two additional divisions. The transduced cells were then harvested, counted, and plated out as single cell clones in four 12-well plates. The single cell clones were selected by culturing in DMEM+10% fetal calf serum containing 400 μg/ml of G418. Single cell colonies began to appear after 2-3 weeks in culture. These colonies were first expanded to a single T-25 flask, and then expanded to two T-150 flasks which were grown to 90% confluency. The first flask was harvested for storage of cells in liquid nitrogen, and the second flask was processed for total RNA.

Alternatively, myoblasts are transducible by direct incubation with plasmids containing bone morphogenetic protein genes (e.g., mouse BMP-4, Fang et al., 1996, Proc. Natl. Acad. Sci. US.A. 93:5753-5758; human BMP-1, BMP-2A and BMP-3, Wozney et al., 1988, Science 242:1528-1532; human BMP-4, Ahrens et al., 1993, DNA and Cell Biology 32:871-880). For example, myoblasts may be successfully transduced by standard calcium phosphate coprecipitation or lipofection.

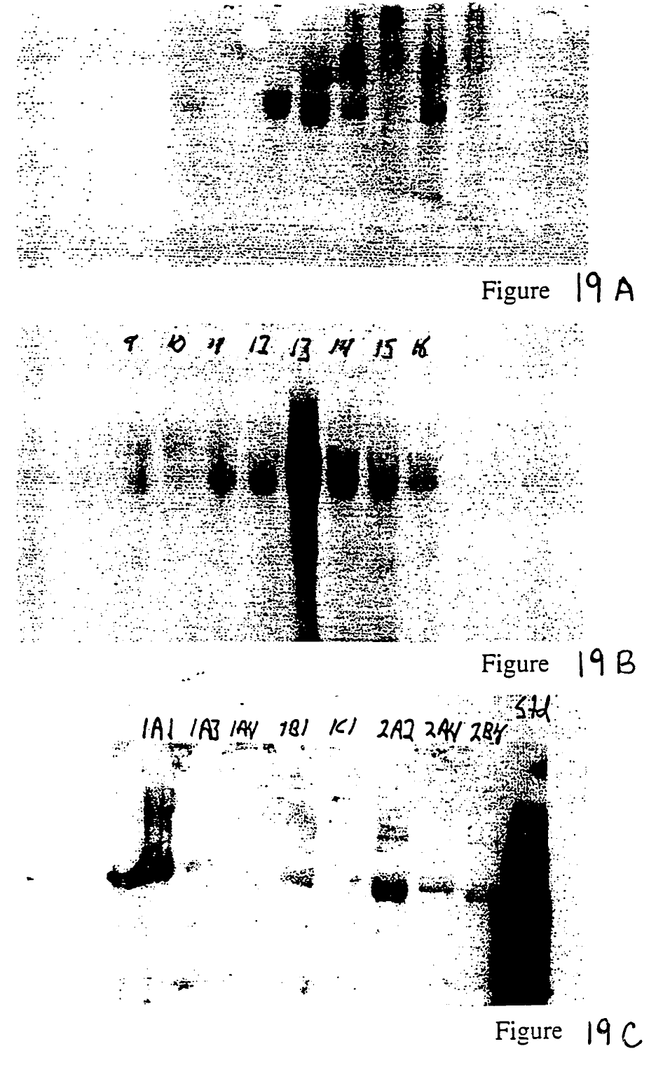

Northern blot analysis was performed on the cell clones with 20 μg of total or standard RNA per lane (FIGS. 19A-C). The blots were hybridized with a cDNA probe to rhBMP-6 (supplied by Genetics Institute, Cambridge, Mass.). Referring to FIGS. 19A and B, clones expressing high levels of rhBMP-6 mRNA (e.g., cell line 4A1 in lane 13 of FIG. 19B) were expanded and recloned from single cell colonies. Referring to FIG. 19C, subclones of cell line 4A1 were rescreened by Northern blot analysis, and clones 1A1 and 2A2 expressed high levels of rhBMP-6 mRNA relative to the other clones. Cell colonies retaining high expression of rhBMP-6 were harvested and banked in liquid nitrogen.

2. Expression of Biologically Active BMP-6

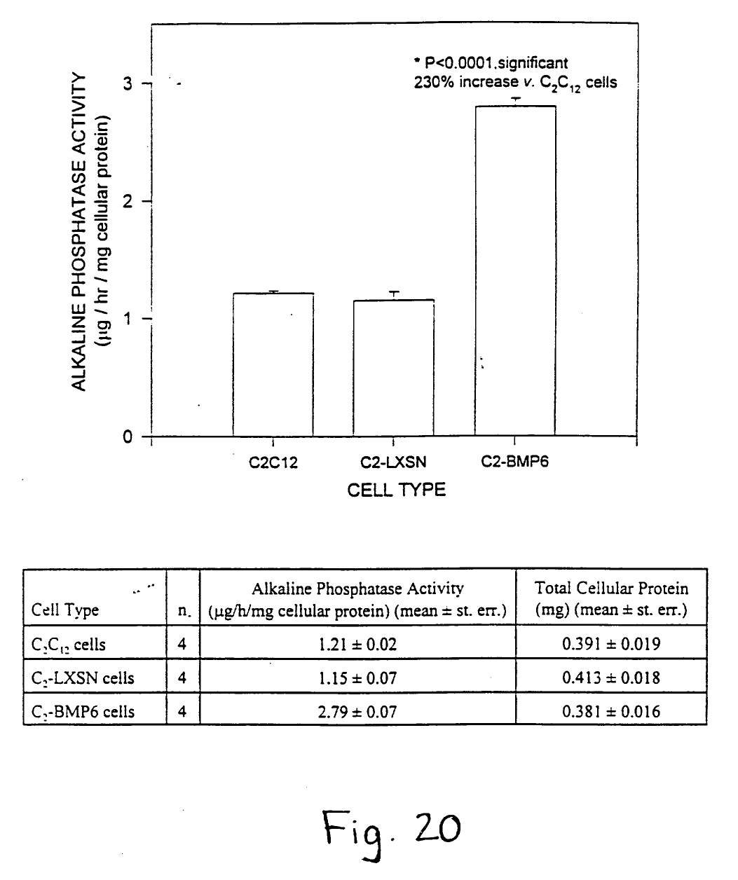

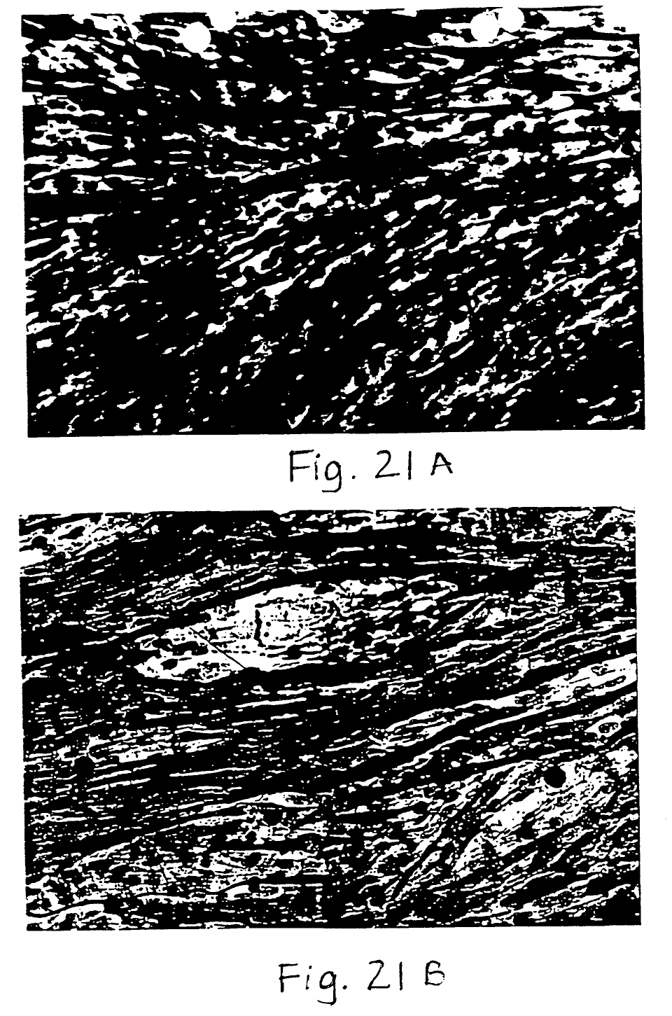

The biological activity of rhBMP-6 in cell colonies retaining high expression of rhBMP-6 (i.e., C₂-BMP6 cells) was determined by measuring alkaline phosphatase activity (i.e., an osteoblastic marker) in the cells after 14 days in culture (FIG. 20). Normal C2C12 cells (i.e., non-transduced cells) and C2C12 cells transduced with the LXSN vector alone (i.e., C₂-LXSN cells) were used as controls.

Cells were harvested after 14 days as follows. Wells containing the cells were rinsed with phosphate buffered saline (0.lm, pH 7.4; PBS) and then typsinized with five drops per well of 0.05% trypsin/EDTA solution in PBS. The trypsin/EDTA was neutralized with 500 μl serum-containing media per well, and cells were transferred to microcentrifuge tubes and centrifuged at 900 rpm for four minutes to pellet the cells. Cell pellets were resuspended and lysed in 500 μl of TXM buffer (10 mM Tris HCL; 1.0 mM magnesium chloride; 0.02 mM zinc chloride; 0.1% Triton X-100; and 0.02% sodium azide), and stored at −20° C. until assayed or assayed immediately for alkaline phosphatase activity as follows.

One hundred microliters of cell lysate, blank (buffer minus substrate), or standard (5 mM p-nitrophenol in buffer) was added to a tube containing 400 μl of alkaline phosphate assay substrate and buffer (0.1 mg glycine; 2.0 mM magnesium chloride; 2 mg/ml p-nitrophenyl phosphate) and incubated at 37° C. for 30 min. The reaction was stopped by adding 500 μl of 0.25 N NaOH, and the optical density at 410 nm was read on a spectrophotometer. The total cellular protein in each sample was measured with a Bio-RadTM protein assay essentially according to the manufacturer's instructions (Bio-Rad Laboratories, Hercules, Calif.) and alkaline phosphatase activities calculated as follows:

$$\text{Total Alkaline Phosphatase Activity for Sample} [ \frac{μ g}{hour} ] = \frac{( 2 \times \text{Sample Optical Density×Dilution Factor} )}{( \text{Average of Standard Optical Densities} )}$$

$$\text{Alkaline Phosphatase Activity} [ \frac{μ g / hour}{\text{mg cellular protein}} ] = \frac{\text{Total Alkaline Phosphatase Activity for Sample}}{\text{Total Cellular Protein for Sample}}$$

3. Delivery of BMP-6 by Implanting Skeletal Muscle Organoids

The ability of C₂-BMP6 cells to differentiate and fuse to form skeletal muscle myofibers was analyzed by morphometric analysis and expression of the muscle-specific protein sarcomeric tropomyosin after six to fourteen days in culture. Normal C2C12 cells and C₂-LXSN cells were used as controls.

Normal C2C12 cells, C₂-LXSN cells, and C₂-BMP6 cells were cultured separately in T-75 flasks. At 80% confluence, all cell types were individually subcultured and plated into four well-plates (i.e., 15-mm diameter wells pretreated with a collagen spray 1 mg/ml of rat-tail collagen, type I in 1% acetic acid). The cells were plated at a density of 100,000 cells per well in 750 μl of growth medium (DMEM-high glucose; 10% calf serum; 10% fetal calf serum; 100 units/ml penicillin; and 0.1 mg/ml streptomycin) and incubated in a humidified, 37° C., 5% CO₂ atmosphere.

The cells were fed 750 μl warm growth medium per well every 48 hours (i.e., day 2 and day 4 post-plating). Five days post-plating when all groups showed 100% confluence, the cells were switched to a low serum fusion medium to promote fusion (DMEM-high glucose; 2% horse serum; 100 units/ml penicillin; 0.1 mg/ml streptomycin). The cells were fed fusion medium on days six, eight and ten post-plating. On day 12 post-plating, the cells were switched to a maintenance medium (DMEM-high glucose; 10% horse serum; 5% fetal calf serum; 100 units/ml penicillin; and 0.1 mg/ml streptomycin). The experiment was terminated on day 14.

Plates were fixed for morphometric analysis 6, 8, 12 and 14 days post-plating as follows. Cells were quickly rinsed twice with Eagle's balanced salt solution (EBSS), fixed with HistochoiceTM for thirty minutes at room temperature, and incubated twice for ten-minutes in EBSS. The samples were then stored in fresh EBSS at 4° C. until used for immunohistochemical analysis.

From storage, samples were warmed to room temperature and rinsed with phosphate buffered saline (PBS; 10 mM, pH 7.4). Samples were then incubated with the primary antibody, anti-sarcomeric tropomyosin (1:100 dilution) in 0.5% Tween 20/PBS for thirty minutes at room temperature, followed by PBS rinsing. Secondary antibody and avidin biotinylated enzyme steps were performed essentially according to the Vectastain(Elite ABC Kit protocol. Samples were then developed with diaminobenzidine tetrahydrochloride (DAB) reagent to produce a brown precipitate, and then lightly counterstained with hematoxylin.

Referring to FIGS. 21A (Day 8 post-plating) and 21B (Day 14 post-plating), the ability of C₂-BMP6 cells to differentiate and fuse to form skeletal muscle myofibers is demonstrated by morphometric analysis (i.e., the presence of longitudinally-oriented multinucleated fibers) and by the presence of sarcomeric tropomyosin (i.e., a muscle-specific protein expressed in differentiated skeletal muscle myofibers but not in undifferentiated, proliferative myoblasts). Because the expression of a biologically active bone morphogenetic protein does not impair the ability of skeletal muscle myoblasts to differentiate and fuse to form skeletal muscle myofibers, skeletal muscle organoids which express bone morphogenetic proteins are produced as described above (see Section I), and are used to deliver bone morphogenetic proteins to an organism also as described above (see Section II).

Because bone morphogenetic proteins are extracellular molecules, skeletal muscle organoid delivery of the protein may be through endocrine, autocrine, or paracrine mechanisms. In a preferred embodiment, the organoid may function as a paracrine organ to deliver a bone morphogenetic protein to chondroblastic or osteoblastic precursor cells. For example, a skeletal muscle organoid expressing a bone morphogenetic protein may be implanted adjacent a non-union fracture to stimulate endochondral bone formation and repair. Alternatively, a skeletal muscle organoid could be implanted in an organism adjacent skeletal tissues which are susceptible to degeneration and fracture consequent to aging (e.g., the hip joint or spinal column of elderly humans). Similarly, bone morphogenetic protein expressing organoids may be employed to treat systemic or regional osteoporosis (e.g., of the spine, femoral neck, and scapular regions of elderly humans). Skeletal muscle organoids expressing bone morphogenetic proteins may also function to accelerate cartilage repair and the healing of segmental defects or bony fusions.

### EXAMPLE 22

Transduction of C2C12 Muscle Cells to Secrete rHIGF-1.

C2C12 mouse myoblasts were transduced with the MFG-IGF-1 retroviral transduction vector. The vectors described herein contain the gene of interest (rhGH or rhIGF-1) under the control of the viral Long Terminal Repeat promoter.

Utilizing an immunocytochemical staining technique for IGF-1, approximately 60% of the cells were transduced. The transduced cells were differentiated into muscle fibers and found to secrete 10 fold higher levels of IGF-1 than nontransduced cells (6.05±1.3 versus 0.49±0.11 ng/mL, P<0.05). These data shown the ability to genetically engineer myoblasts to secrete therapeutic proteins other than rhGH.

### EXAMPLE 23

Human Myoblast Isolation, Tissue Culturing, and Organoid Formation using Adult Human Biopsied Skeletal Muscle.

Standard muscle biopsies were performed on two adult male volunteers and myoblasts isolated by standard tissue culture techniques (Webster et al., 1990, Somatic Cell and Mol. Gen. 16:557-565). One hundred muscle stem cells (myoblasts) were identified from each biopsy by immunocytochemical staining with an antibody against desmin and the myoblasts were expanded through at least 30 doubling. The 100 myoblasts could thus be expanded into greater than 50 billion cells (5×10¹⁰). If these adult human myoblasts are transduced with the MFG-hGH retroviral vector to the same efficiency as the adult rat myoblast shown above, approximately 1×10⁸ of these human myoblasts would be required to raise steady state human serum levels of rhGH to 5-7 ng/mL, a level equivalent to that found in normal adults (Harvey et al., 1995, Growth Hormone Release:Profiles., S. Harvey, C. G. Scanes and W. H. Daughaday, eds., CRC Press, Boca Raton, 193-223). In contrast, GH-deficient elderly have basal GH serum levels around 1.5 ng/mL (Harvey et al., supra, pp.193-223). This is well within the organoid technology's capability.

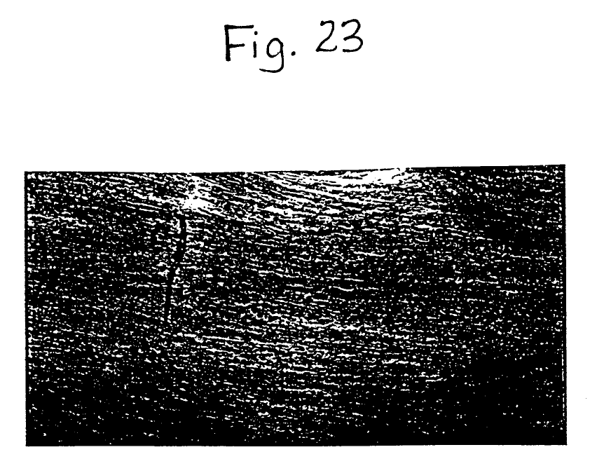

Two million adult human myoblasts were tissue engineered into human organoids (H-organoids) which were very similar in appearance to the C2-organoids and R-organoids described above (FIG. 23). These H-organoids can be maintained in vitro for at least 2 weeks.

### EXAMPLE 24

Transduction of Fetal Human Myoblasts with MFG-HGH.

Human fetal skeletal myoblasts were purchased from a commercial source (Clonetics, Inc.) and transduced with MFG-hGH retroviral expression vector. The myoblasts were differentiated into myofibers and their secretion of rhGH assayed over an 11 day period. The cells secreted very high levels of rhGH (2-3 ug rhGh/10⁶ cells/day), which were equivalent to the rate of secretion by the C2C12 myoblasts used previously for in vivo attenuation of skeletal muscle wasting. These data lead one of skill in the art to conclude that human myoblasts can be genetically engineered to secrete therapeutic levels of rhGH.

### EXAMPLE 25

R-organoids Survive when Implanted Subq into Inbred Fisher 344 Adults.

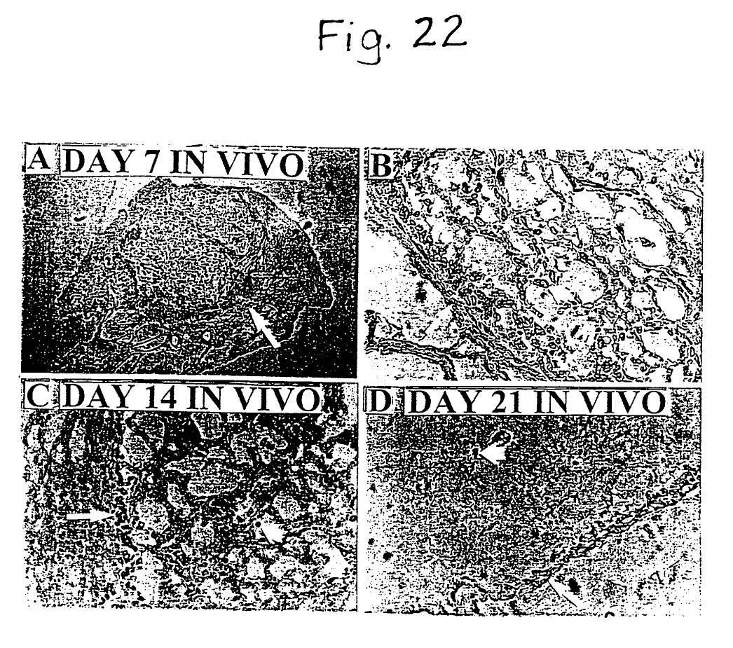

We have found that differentiated R-organoid myofibers implanted subQ into adult Fisher 344 rats survive for at least five weeks in vivo (FIG. 22). Myofiber survival is greatest on the surface of the implant (FIG. 22D), probably because capillaries do not infiltrate into the interior of the R-organoids until after four weeks (data not shown). By five weeks in vivo, the surface myofibers in the organoids have hypertrophied at least 3-fold compared to three week myofibers (data not shown). One possible mechanism to stimulate more rapid capillary in-growth into the R-organoids is by expressing vascular endothelial growth factor (VEGF) in the R-organoids using transient transfections with VEGF plasmids (Tsurumi et al., 1996, Circulation 94:3281-3290).

### EXAMPLE 26

Treatment of Wasting Cachexia with Growth Hormone, Insulin and/or Insulin-like Growth Factors Delivered from Implanted Postmitotic Organized Tissue Constructs

The goal of this study is to utilize postmitotic organoids genetically engineered to secrete therapeutic levels of recombinant proteins such as growth hormone, insulin and/or somatostatin to Lewis/Wistar Rats.

Postmitotic organoids genetically engineered to secrete therapeutic levels of insulin (GenBank Accession #2098404), growth hormone (GenBank Accession # 134728) and somatostatin (GenBank Accession #349927) are used to treat wasting cachexia in Lewis/Wistar rats. Cells (e.g. myoblasts or fibroblasts) are isolated from animals and plated separately in tissue culture flasks. When the cells are nearly confluent they are harvested and plated at low density in tissue culture plates. The low density cultures are transduced with a retroviral vector containing the gene for insulin, growth hormone or somatostatin, as described in Example 2. Transduced cells are engineered into organized tissue constructs as described in Example 1. Organized tissue constructs are implanted into rats as described in Example 3. In vivo serum levels of insulin, growth hormone or somatostatin are measured at varying times after implantation. The in vivo serum samples are collected by tail bleeds and the levels of insulin, growth hormone or somatostatin are determined by radioimmunoassay.

A given treatment for wasting cachexia according to the invention may be tested in an art accepted animal model of wasting cachexia by implanting the organized tissue producing a recombinant protein (e.g. growth hormone, insulin or somatostatin) into the diseased animal and observing clinical parameters over time. An art-accepted animal model of wasting cachexia is provided by Lewis/Wistar rats that have been subcutaneously inoculated with the MAC-33 tumor.

According to this model, Lewis/Wistar rats (175-200 g) were inoculated subcutaneously on the left flank with 1.0×10⁶ tumor cells in single cell suspension. This tumor (MAC-33) is a mammary adenocarcinoma that metastasizes spontaneously to regional lymph nodes and lungs after subcutaneous implantation. The MAC-33 tumor is a variant of the nonmetastasizing AC-33 tumor originally induced in this strain of rat with the alkylating agent, dimethyl-β-aziridinopropionamide. The MAC-33 tumor causes host weight loss approximately 25 days after implantation, with no anorexia until just before death. Host death occurs 45-50 days after tumor implantation by local tumor invasion, sepsis, or massive pulmonary metastasis (Bartlett et al., supra). Upon treatment of these rats with growth hormone, insulin and/or somatostatin these rats demonstrated increased body weight and muscle size, as compared to control animals that experienced weight loss over the same period (Bartlett et al.,supra).

Therapeutic efficacy of treatment of wasting cachexia according to the invention by implantation of an organized tissue producing growth hormone, insulin or somatostatin as described herein, is indicated by changes in clinical parameters such as changes in body weight, muscle size, and weight and total muscle protein (at least 5-10% and preferably 25-60%), as compared to control animals that experienced weight loss over the same time period.

Human patients suffering from wasting cachexia may be treated accordingly by implanting organoids producing growth hormone, insulin or somatostatin, measuring the level of these recombinant proteins, determining changes in body weight, muscle size and total muscle protein, and the amelioration of symptoms associated with wasting cachexia over time.

I. Neurological Disorders

The invention also provides methods of treating neurological disorders, including peripheral neuropathy, injury, and neurodegenerative diseases (e.g. Parkinson's disease, Huntington's disease or Alzheimer's disease).

Peripheral Neuropathy/Injury

Peripheral neuropathy refers to a malfunction of the peripheral nerves that can disrupt sensation, muscle activity or the function of internal organs. Peripheral neuropathy can involve damage to a single nerve (mononeuropathy), two or more nerves (multiple mononeuropathy) or multiple nerves simultaneously (polyneuropathy). Mononeuropathy is most commonly caused by physical injury and includes carpal tunnel syndrome, ulnar nerve palsy, radial nerve palsy and peroneal nerve palsy. Polyneuropathy is caused by numerous factors including bacterially produced toxins, autoimmune reactions, toxic agents, cancer, nutritional deficiencies and metabolic disorders. Chronic polyneuropathy can result from a number of disorders including diabetes, kidney failure, and malnutrition and the treatment of polyneuropathy depends on the cause (Berkow et al., supra).

Neuronal Disease and Injury

Every year, hundreds of thousands of patients are treated for neurodegenerative disease (e.g. Parkinson's disease, Huntington's Disease, Alzheimer's, multiple sclerosis) or traumatic injury. Damage to the Peripheral Nervous System (PNS) and the Central Nervous System (CNS) can lead to serious disability and death. Therefore, PNS and CNS damage and the attendant social and economic costs are staggering. The adult PNS retains some capacity for regeneration following injury but the return of function in the clinical setting is quite variable and motor and sensory deficits (paralysis, weakness, numbness, etc.) invariably persist (Dyck and Thomas, eds. Peripheral Neuropathy, 3rd. Ed., 1993; W. B. Saunders, Philadelphia, Pa.). In certain situations wherein neuropathy is caused by an underlying disease, such as diabetes or is a drug-induced neuropathy, or in cases where extensive damage has occurred due to severe nerve defects or crush and avulsion injuries, recovery is negligible. Repair of the diseased or damaged CNS, which includes the brain and spinal cord, represents an even greater challenge since almost all disease and injuries lead to an irreversible loss of function (memory loss, loss of motor function, etc.) (Bjorklund et al., eds., 1990, Brain Repair, Stockton Press, New York, N.Y.). New strategies to optimize and enhance regeneration include the delivery of growth-promoting molecules, generally called nerve growth factors.

Delivery of Nerve Growth Factors:

Growth or neuronotrophic factors produced by support cells (e.g. Schwann cells, oligodendrocytes) or by target organs (e.g. muscle fibers, connected neurons) ensure the survival and general growth of neurons. Some factors support neuronal survival, others support nerve outgrowth, and some do both. Numerous growth factors have been identified, cloned, and some have been synthesized through recombinant technologies (Barde, 1989, Neuron 2:1525). The clinical use of such agents has been limited by an inability to deliver the growth factors to the nervous system in the appropriated dose and over an appropriate time period. Methods of administering growth factors by single or multiple injections of growth factors have disadvantages including early burst release, poor control over local drug levels, and significant side effects. A tissue-based delivery system offers the advantages of allowing for controlled regulation of the rate and amount of factor release and maintaining delivery for an extended time period (several months or longer) if needed (e.g. for degenerative diseases such as Parkinson's)

GROWTH FACTORS USEFUL FOR NEURAL REPAIR

| Growth Factor | Reported Function(s) |
|---|---|
| Neural factors: | |
| NGF - nerve growth factor | Neuronal survival, |
| | Axon-Schwann cell |
| | interaction |
| BDNF - brain-derived neurotrophic factor | Neuronal survival |
| CNTF - ciliary neuronotrophic factor | Neuronal survival |
| GDNF - glia-derived neurotrophic factor | Neuronal survival |
| GGF - glial growth factor | Schwann cell mitogen |
| NT-3 - neurotrophin 3 | Neuronal survival |
| NT-4/5 - neurotrophin 4/5 | Neuronal survival |
| General factors: | |
| IGF-1 - insulinlike growth factor 1 | Axonal growth, Schwann cell |
| | migration |
| IGF-2 - insulinlike growth factor 2 | Motoneurite sprouting, muscle |
| | reinnervation |
| PDGF - platelet-derived growth factor | Cell proliferation, neuronal |
| | survival |
| aFGF - acidic fibroblast growth factor | Neurite regeneration, cell |
| | proliferation |
| bFGF - basic fibroblast growth factor | Neurite regeneration, |
| | neovascularisation |

Tissue-based delivery may also be used for the concurrent release of growth factors which preferentially control the survival and outgrowth of motor and sensory neurons. For example, NGF and b-FGF control sensory neuronal survival and outgrowth and brain derived growth factor (BDGF) and ciliary neuronotrophic factor (CNTF) control motor neuronal survival and outgrowth. Other molecules, NT-3 and NT 4/5 may carry out both functions. Factors which promote Schwann cell proliferation (e.g. glial growth factor, GGF) may also be useful in enhancing nerve growth. Growth factors released in a sustained, physiologic manner by tissue-based implants may allow regeneration in cases where large nerve deficits exist and in sites where regeneration does not normally occur (e.g. brain and spinal cord).

Animal Models for PNS and CNS Repair

Numerous animal models for neural disease have been developed. Nerves of the PNS can be cut or crushed in a model of nerve transection or neuropathy. It has been demonstrated that nerve guidance channels designed to slowly release basic fibroblast growth factor (bFGF) or nerve growth factor (NGF) can support regeneration over a critical nerve gap in a rat model (Aebischer et al., 1989, J. Neurosci. Res., 23:282-289, Derby et al., 1993, Exp. Neurol., 119:176-191).

According to the method of Aebischer et al, the left sciatic nerve of Nembutal-anesthetized rats (30 mg/kg) was exposed through a skin incision along the anterior medial aspect of the thigh after retracting the gluteus maximus muscle. The sciatic nerve was mobilized from the ischial tuberosity to the tibial-peroneal bifurcation by gently dissecting the overlying connective tissue sheaths. An 8 mm segment of the nerve 1 mm proximal to the tibial-peroneal bifurcation was resected and discarded. The proximal and distal nerve stumps were secured within the 19 mm long guidance channel lumen with a single 10-0 nylon suture. The nerves were positioned 2 mm from the channel ends, so that the proximal and distal stumps were separated by a gap of 15 mm. The surgical site was irrigated with sterile saline. Muscle approximation and skin closure was then achieved with 6.0 monofilament nylon (Ethilon®) and 6.0 braided silk sutures. Aseptic surgical techniques were maintained throughout the procedure, which was performed with the aid of a Zeiss operating microscope. Animals were implanted for 4 weeks with channels made of pure ethylene-vinyl acetate copolymer pellets (EVA), EVA/BSA, EVA/CytC, EVA/BSA/b-FGF, EVA/BSA/denatured b-FGF, EVA/BSA/α1-GP, and EVA/BSA/b-FGF/α1-GP (Aebischer et al., 1989, supra).

In the CNS nerve structures can be cut or chemical substances can be administered to achieve neural damage (Emerich et al., 1994, Neuro. Methods, 21:65-133, Aebischer et al., 1994, Exp. Neurol., 126: 151-158).

### EXAMPLE 27

Treatment of Neurological Disorders with Nerve Growth Factors Delivered from Implanted Organized Tissue Constructs

Primary cells are isolated from the thigh muscles of 12 week old CD rats, and genetically engineered to secrete NGF, CNTF, or bFGF (or other molecules if desired). Cells are expanded in culture until nearly confluent, and organoids are formed as described in Example 1. Smaller organoids are formed by suspending 0.5×10⁶ cells in a 100 μL solution of collagen (1.6 mg/ml growth medium): Matrigel™ (6:1) and casting the mixture into silicone rubber molds, 2 mm i.d.×5 mm long. Organoids can be fabricated to contain a pure or mixed population of fibroblasts and fused myofibers, wherein both cell types are aligned parallel to the long axis of the mold. Cells secreting one or more growth factors can also be used. Secretion of growth factors can be assessed at various time points using ELISA kits for NGF, CNTF, bFGF, etc (R&D Systems). Organoids are implanted under tension into groups of rats that will have received; 1) sciatic nerve transection (to simulate nerve injury), 2) sciatic nerve crush (to simulate peripheral neuropathy), 3) ablation of the fimbria-formix (to simulate Alzheimer's lesion) or 4) 6-OH dopamine unilaterally (to simulate hemiparkinsonian symptoms). Rats from groups 1) and 2) are transplanted with larger organoids as described in Example 3. Rats from groups 3) and 4) are implanted with smaller organoids which are implanted by anesthetizing rats with sodium nembutal (55 mg/kg IP), placing the rats into a stereotactic device, shaving and sterilizing the skull and making a 10 mm circular hole along the midline of the skull. Implants are placed in the ventricular system and parenchymal tissue with the use of stereotactic guidance. All wounds are closed with standard two layer closure. Other neural disorders can potentially be treated with organized tissue constructs genetically engineered to secrete the relevant molecules required for treatment (as listed in the above table).

A given treatment for a neurological disorder according to the invention may be tested in an art accepted animal model of a neurological disorder by implanting the organized tissue producing a recombinant protein (e.g. NGF, CNTF, or bFGF) into the diseased animal and observing clinical parameters over time. Art-accepted animal models of neurological disorders include but are not limited to models wherein CNS nerve structures are cut or chemical substances are administered to achieve neural damage (Emerich et al., supra, Aebischer et al., supra).

According to one animal model of a neurological disorder, a hemiparkinsonian model was created by unilateral intracarotid injection of 0.3 to 0.6 mg/kg of 1-methyl-4-phenyl-1 ,2,3,6-tetrahydropyridine (MPTP) in approximately 15 cc of 0.9% normal saline at a rate of 1.0 ml/min. Sterile, open microsurgical procedures were performed to allow retrograde injection of the MPTP solution through 26-gauge needles placed in the right common carotid artery after permanent ligation of the external carotid artery and its proximal branches. (Aebischer et al., 1994, supra).

Therapeutic efficacy of treatment of neurological disorders according to the invention by implantation of an organized tissue producing a bioactive molecule useful for the treatment of a neurological disorder (e.g. NGF, CNTF or bFGF) as described herein, is indicated by changes in clinical parameters such as peripheral nerve regeneration (at least 5-10% and preferably 15-100%). Histological tissue analysis (e.g. increased number and diameter of nerve fibers) and functional assays (e.g. increased nerve conduction velocities) can be used to detect peripheral nerve regeneration in response to a recombinant protein. Correction of central nervous disorders by recombinant proteins can be determined histologically (e.g. increased number of neurons) and functionally (e.g. improved performance on memory or motor coordination tests).

Human patients with neurological disorders may be treated accordingly by implanting organoids producing a recombinant protein (e.g. NGF, CNTF or bFGF), measuring the level of these recombinant proteins, determining if nerve regeneration has occurred, determining changes in the number of neurons or neuron function, and the amelioration of symptoms associated with neurological disorders over time.

J. Skin Disorders

The invention also provides methods of treating skin disorders including wound healing and ulcers.

Wound Healing

Wound healing involves a complex process of cell migration and proliferation, synthesis of extracellular matrix, angiogenesis and remodeling of the collagenous framework that requires many growth factors, such as TGF-beta and platelet-derived growth factor (Amento et al., 1991, Ciba Foundation Symposium, 157: 115-123, Hosgood et al., 1993, Vet. Surg., 226: 490-495. Rat and rabbit animal models for wound healing have been demonstrated (Terrell et al., 1993, International Review Exp Pathology, 34 Pt B: 43-67).

Ulcers

An ulcer is a hole that extends through tissue such as the muscularis mucosa into the submucosa (or a deeper layer) of the gastrointestinal tract. The combined action of acid and pepsin is more injurious to vulnerable mucosa than that of either agent alone. Smoking, stress, heredity factors, aspirin/non-steroidal anti-inflammatory drugs and/or infection with Campylobacter pylori are known to cause peptic ulcers (Chopra et al., 1989, Pathophysiology of Gastrointestinal Diseases). Treatment of peptic ulcers with recombinant proteins such as epidermal growth factor (EGF) may assist in protecting, repairing and healing gastroduodenal mucosa. In an animal model of ulcers, acetic acid has been used to ulcerate rats (Uchida et al., 1989, Japan Journal of Pharmacology, 50:366-368). Ulcers can also be formed in other tissues such as nonhealing skin ulcers in diabetic patients and venous ulcers (Nath et al., 1998, Acta Haematol., 99:175 and Vowden, 1998, J. Wound Care 7:143).

### EXAMPLE 28

Acceleration of Wound Healing with Growth Factors Delivered from Implanted Organized Tissue Constructs

Treatment of non-or slow-healing wounds with growth factors delivered from implanted nonproliferative organized tissue constructs may accelerate the process of wound healing. Organized tissue constructs genetically engineered to secrete therapeutic levels of recombinant proteins such as TGF-beta and/or platelet-derived growth factor are used to deliver sustained levels of these growth factors to increase rate the of healing and tensile strength of the repaired tissue.

Cells (e.g. myoblasts or fibroblasts) are isolated from animals and plated separately in tissue culture flasks. When the cells are nearly confluent they are harvested and plated at low density in 35 mm diameter tissue culture plates. The low-density cultures are transduced with the MFG-retroviral vector containing the gene for recombinant protein (e.g. human TGF-β, GENBANK Accession # 339558; PDGF, GENBANK Accession # 494431) as described in Example 2. Transduced cells are tissue engineered into organized tissue as described in Example 1. In vitro, transduced cells in organoids should secrete significantly greater amounts of recombinant protein as compared to nontransduced controls. One or more human recombinant protein secreting constructs are implanted under tension in mice (as described in Example 1) or in rats (as described in Example 3). The in vivo serum levels of recombinant proteins are measured at varying times after implantation (by standard radioimmunoassay and ELISA) and should be significantly increased as compared to the in vivo serum levels of animals implanted with non-recombinant protein secreting tissue constructs.

A given method for accelerating wound healing according to the invention may be tested in an art accepted animal model of wound healing by implanting the organized tissue producing a recombinant protein (e.g. TGF-β or PDGF) into the diseased animal and observing clinical parameters over time. Art-accepted rat and rabbit models of wound healing have been established (see Terrell et al., supra). According to these models, wounds can be created in rats by using an 8 mm diameter Baker/Cumins biopsy punch or in rabbits by surgical methods (Terrell et al., supra).

Therapeutic acceleration of wound healing according to the invention by implantation of an organized tissue producing TGF-β or PDGF as described herein, is indicated by changes in clinical parameters such as changes in the rate of wound healing and an increased strength of healing of wounds that are difficult to heal (at least 5-10% and preferably 25-100%). Methods for measuring the rate and strength of wound healing can be found in Reid, 1997, Am. J. Obstet. Gynecology and Disa et al., 1993, Plast. Reconstructive Surgery, 92:884.

Wound healing in human patients may be treated/accelerated accordingly by implanting organoids producing TGF-P or PDGF, measuring the level of these recombinant proteins, determining changes in the rate of wound healing and the strength of healing of wounds, and the amelioration of symptoms associated with unhealed wounds over time.

### EXAMPLE 29

Treatment of Ulcers with Recombinant Proteins Delivered from Implanted Organized Tissue Constructs

Organized tissue constructs genetically engineered to secrete therapeutic levels of recombinant proteins such as EGF are used to enhance the healing process in chronic ulcer patients.

Cells (e.g. myoblasts and fibroblasts) are isolated from animals and plated separately in tissue culture flasks. When the cells are nearly confluent they are harvested and plated at low density in 35 mm diameter tissue culture plates. The low-density cultures are transduced with the MFG-retroviral vector containing the gene for a recombinant protein (e.g. EGF, GENBANK Accession #119226) as described in Example 2. Transduced cells are tissue engineered into organized tissue constructs as described in Example 1.

It is expected that transduced cells in organoids will secrete significantly greater amounts of EGF than nontransduced control constructs, in vitro. One or more EGF secreting constructs are implanted under tension in mice (as described in Example 1) or in rats (as described in Example 3). The in vivo, EGF serum levels are measured at varying times after implantation, by standard radioimmunoassay and ELISA, and are expected to show a significant increase as compared to the levels in animals implanted with non-EGF secreting tissue constructs.

A given treatment for ulcers according to the invention may be tested in an art accepted animal model of ulcers by implanting the organized tissue producing a recombinant protein (e.g. EGF) into the diseased animal and observing clinical parameters over time. Art-accepted animal models of ulcers include but are not limited to rats that have been treated with acetic acid to induce ulceration (Uchida et al., supra).

According to the method of Uchida et al., ulcers were induced by acetic acid (20%, 0.05 ml) in Sprague-Dawley strain (Slc:SD) male rats weighing from 220 to 240 g (7 weeks). Ulcer-size [Ulcer index (UI)=length (mm)×width (mm)] was determined, and cumulative healing and relapse rates and the level of prostaglandin E (PGE) was measured. To determine the PGE level, a [³H]-Prostaglandin E Radioimmunoassay Kit (Clinical Assays, Division of Travelol Laboratories, Inc.) was used (Uchida et al., supra).

Therapeutic efficacy of treatment of ulcers according to the invention by implantation of an organized tissue producing EGF as described herein, is indicated by changes in clinical parameters such as changes in the rate (at least 5-10% and preferably 25-100%) at which morphological repair of the wound site occurs. Methods for measuring the rate of wound repair can be found in Slomiany et al., 1997, Gen. Pharmacology, 29:367 and Slomiany et al., 1997, Scand. J. Gastroenterology, 32:873.

Human patients with ulcers may be treated accordingly by implanting organoids producing EGF, measuring the level of EGF, determining changes in the rate at which morphological repair of the wound site occurs, and the amelioration of symptoms associated with ulcers over time.

### Dosage and Therapy

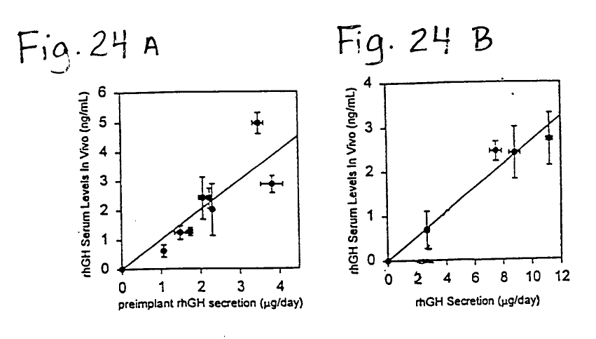

One of the major disadvantages of delivery of foreign proteins produced from injected genetically engineered cells is the great variability in the number of cells which survive from individual to individual and therefore the unpredictability of the delivery dose. The invention confers an advantage in terms of predictability of dosage. With genetically engineered organoids, the protein secretion levels can be monitored preimplantation in vitro. Accurate correlations can be made on in vivo serum levels of a bioactive compound (e.g. rhGH) based on the preimplantation in vitro organoid (e.g. C2-organoid) secretion levels (FIG. 24). In order to correlate the delivery dose of an organoid implanted in vivo for treatment according to the invention, organoid protein secretion levels (e.g., C2-organoid rhGH) can be varied by engineering a protein-producing organoid (e.g., C2-organoids) with different numbers of protein-secreting myofibers. In addition, varying numbers of organoids can be implanted and levels of bioactive compound determined. For C2-organoids, one to four organoids were implanted per animal, and a corresponding increase in the level of bioactive compound (rhGH) was found. Therefore, two protocols are provided for controlling protein delivery dose from organoids over an approximately 10 fold range; i.e., the selection of a number of bioactive compound-secreting cells for implantation and the selection of a number of bioactive compound secreting organoids for implantation. In FIG. 24 (A and B), therefore, a correlation is shown of in vivo rhGH serum levels from rhGH levels secreted in vitro. A linear relationship exists for the amount of rhGH secreted by C2-organoids preimplantation and postimplantation.

The invention is applicable to therapies in which one or more bioactive compounds are delivered to an organism, for example, a mammal in therapeutically effective levels. A therapeutic gene is one which is expressible in a mammalian, preferably a human, cell and encodes RNA or a polypeptide that is of therapeutic benefit to a mammal, preferably a human. A vector may also include marker genes, such as drug resistance genes, the β-galactosidase gene, the dihydrofolate reductase gene, and the chloramphenicol acetyl transferase gene. A therapeutic effect is evident, for example, where the therapeutic gene encodes a product of physiological importance, such as replacement of a defective gene or an additional potentially beneficial gene function, and is expected to confer long term genetic modification of the cells and be effective in the treatment of disease.

As discussed above, the dosages of a bioactive compound administered according to the invention will vary from patient to patient; a “therapeutically effective dose” will be determined by the level of enhancement of function of the transferred genetic material balanced against any risk or deleterious side effects. Monitoring levels of gene introduction, gene expression and/or the presence or levels of the encoded product will assist in selecting and adjusting the dosages administered. Generally, a composition including a bioactive compound-producing organoid according to the invention will be administered in a single dose (per time period in which the organoid implant is judged to be effective in producing the bioactive compound), such that the bioactive compound is produced in the mammal in the range of 1 ng -100 ug/kg body weight, preferably in the range of 100 ng -10 ug/kg body weight, depending upon the nature of the bioactive compound, its half-life, and its biological effect.

### Other Embodiments

The above description is not intended to limit the invention either in spirit or scope. Other embodiments are within the following claims.

What is claimed is:

1. A method of delivering a bioactive compound to an organism, comprising the steps of:
growing a plurality of cells in vitro under conditions that allow the formation of an organized tissue having an in vivo-like gross and cellular morphology and comprising substantially post-mitotic cells, at least a subset of said cells containing a foreign DNA sequence which mediates the production of a bioactive compound; and
implanting said tissue into said organism, whereby said bioactive compound is produced and delivered to said organism, whereby said bioactive compound is of a type or is produced in an amount not produced by a tissue lacking said foreign DNA sequence.

2. A method of providing a bioactive compound to an organism in therapeutic need thereof, comprising:
implanting into an organism an organized tissue having an in vivo-like gross and cellular morphology and comprising substantially post-mitotic cells, wherein at least a subset of cells of said organized tissue contain a foreign DNA sequence which mediates the production of a bioactive compound, wherein said bioactive compound is produced in said organism in a therapeutically effective amount.

3. A method of providing a bioactive compound to an organism in therapeutic need thereof, comprising:
implanting into an organism an organized tissue comprising substantially post-mitotic cells and having a three-dimensional cellular organization that is retained upon implantation of said tissue into said organism, wherein at least a subset of cells of said organized tissue contain a foreign DNA sequence which mediates the production of a bioactive compound, wherein said bioactive compound is produced in said organism in a therapeutically effective amount.

4. A method of treating a disease in an organism comprising:
implanting into an organism an organized tissue having an in vivo-like gross and cellular morphology and comprising substantially post-mitotic cells, wherein at least a subset of cells of said organized tissue contain a foreign DNA sequence which mediates the production of a bioactive compound, wherein said bioactive compound is produced in said organism in a therapeutically effective amount.

5. A method of treating a disease in an organism comprising:
implanting into an organism an organized tissue comprising substantially post-mitotic cells and having a three-dimensional cellular organization that is retained upon implantation of said tissue into said organism, wherein at least a subset of cells of said organized tissue contain a foreign DNA sequence which mediates the production of a bioactive compound, wherein said bioactive compound is produced in said organism in a therapeutically effective amount.

6. The method of claim 4 or 5 wherein said disease is a blood disorder.

7. The method of claim 4 or 5 wherein said disease is a bone or joint disorder.

8. The method of claim 4 or 5 wherein said disease is cancer.

9. The method of claim 4 or 5 wherein said disease is a cardiovascular disorder.

10. The method of claim 4 or 5 wherein said disease is an endocrine disorder.

11. The method of claim 4 or 5 wherein said disease is an immune disorder.

12. The method of claim 4 or 5 wherein said disease is an infectious disease.

13. The method of claim 4 or 5 wherein said disease is a wasting disorder.

14. The method of claim 4 or 5 wherein said disease is a neurological disorder.

15. The method of claim 4 or 5 wherein said disease is a skin disorder.
